(19) United States
(12) Patent Application Publication
(10) Pub. No.: US 2025/0127858 A1
(43) Pub. Date: Apr. 24, 2025

(54) NOVEL CYTOKINE PRODRUGS

(71) Applicant: ASKGENE PHARMA, INC., Camarillo, CA (US)

(72) Inventors: Chunxiao YU, Santa Barbara, CA (US); Yuefeng LU, Moorpark, CA (US); Jian-Feng LU, Oak Park, CA (US)

(73) Assignee: ASKGENE PHARMA, INC., Camarillo, CA (US)

(21) Appl. No.: 18/960,740

(22) Filed: Nov. 26, 2024

Related U.S. Application Data
(63) Continuation of application No. 16/979,404, filed on Sep. 9, 2020, now abandoned.
(60) Provisional application No. 62/801,649, filed on Feb. 6, 2019.
(60) Provisional application No. 62/680,707, filed on Jun. 5, 2018.
(60) Provisional application No. 62/644,577, filed on Mar. 18, 2018.
(60) Provisional application No. 62/644,384, filed on Mar. 17, 2018.
(60) Provisional application No. 62/643,104, filed on Mar. 14, 2018.
(60) Provisional application No. 62/640,969, filed on Mar. 9, 2018.

(51) Int. Cl. A61K 38/20; A61K 38/19; A61K 47/64; A61K 47/65; A61K 47/68; A61P 35/00; C07K 14/55

(52) U.S. Cl. CPC A61K 38/2086; A61K 38/195; A61K 38/2013; A61K 47/6425; A61K 47/643; A61K 47/65; A61K 47/6813; A61K 47/6843; A61K 47/6849; A61K 47/6853; A61K 47/6857; A61K 47/6865; A61K 47/6869; A61K 47/6871; A61P 35/00; C07K 14/55

(57) ABSTRACT

Provided herein are cytokine muteins and cytokine prodrugs and methods of making and using thereof.

### CROSS REFERENCE TO RELATED APPLICATIONS

The present application is a continuation of U.S. patent application Ser. No. 16/979,404, filed on Sep. 9, 2020, which is a national stage application under 35 U.S.C. § 371 of International Patent Application No. PCT/US2019/021654, filed Mar. 11, 2019, which claims priority from U.S. Provisional Applications 62/640,969, filed Mar. 9, 2018; 62/643,104, filed Mar. 14, 2018; 62/644,384, filed Mar. 17, 2018; 62/644,577, filed Mar. 18, 2018; 62/680,707, filed Jun. 5, 2018; and 62/801,649, filed Feb. 6, 2019. The contents of the aforementioned priority applications are incorporated herein by reference in their entirety.

### SEQUENCE LISTING

The instant application contains a Sequence Listing which has been submitted electronically in XML format and is hereby incorporated by reference in its entirety. The electronic copy of the Sequence Listing, created on Aug. 29, 2024, is named 025471.JP102.xml and is 401,264 bytes in size.

### BACKGROUND OF THE INVENTION

Interleukin-2 (IL-2) plays a central role in lymphocyte generation, survival and homeostasis. It has 133 amino acids and consists of four antiparallel, amphiphatic alpha-helices that form a quaternary structure essential for its function (Smith, Science 240:1169-76 (1988); Bazan, Science 257:410-13 (1992)).

IL-2 exerts its activities by binding to IL-2 receptors (IL-2R), which consist of up to three individual subunits. Association of the α (CD25 or Tac antigen), β (CD122), and γ (γ_{c}, common γ chain, or CD132) subunits results in a trimeric, high-affinity receptor for IL-2 (K_{D}˜0.01 nM). Dimeric IL-2 receptor consisting of the β and γ subunits is termed intermediate-affinity IL-2R (K_{D}˜1 nM). The α subunit alone forms the monomeric low affinity IL-2 receptor (K_{D}˜10 nM). See, e.g., Kim et al., Cytokine Growth Factor Rev. 17:349-66 (2006)). Although the dimeric intermediate-affinity IL-2 receptor binds IL-2 with approximately 100-fold lower affinity than the trimeric high-affinity receptor, both the dimeric and trimeric IL-2 receptors can transmit signal upon IL-2 binding (Minami et al., Annu Rev Immunol. 11:245-68 (1993)). Thus, it appears that the a subunit, while conferring high-affinity binding of the receptor to IL-2, is not essential for IL-2 signaling. However, the β and γ subunits are essential for IL-2 signaling (Krieg et al., Proc Natl Acad Sci. 107:11906-11 (2010)). The trimeric IL-2 receptor is expressed by CD4⁺FoxP3⁺ regulatory T (T_{reg}) cells. T_{reg} cells consistently express the highest level of IL-2Rα (CD25) in vivo (Fontenot et al., Nature Immunol 6:1142-51 (2005)). The trimeric IL-2 receptor is also transiently induced on conventional activated T cells, whereas in the resting state these cells express only the dimeric IL-2 receptor.

Depending on the objective, muteins of IL-2 have been made to have either enhanced or reduced binding affinity for CD25. Based on published crystal structures of IL-2/IL-2R complexes, the mutations are often made in or near areas of IL-2 known to be in close proximity to CD25 (Wang et al., Science 310:1159-63 (2005)). IL-2 residues K35, R38, F42, K43, F44, Y45, E61, E62, K64, P65, E68, V69, L72, and Y107 are believed to be in contact with CD25 (U.S. Pat. 9,732,134).

In order to reduce the side effects of IL-2 therapeutics, researchers have mutated IL-2 to reduce its binding affinity for CD25. For example, WO 2008/0034473 refers to mutations R38W and F42K, while WO 2012/107417 refers to mutation at position 72. U.S. Pat. Pub. 2003/0124678 refers to introducing the R38W mutation to eliminate IL-2's vasopermeability activity. Heaton et al. (Cancer Res. 53:2597-602 (1993); U.S. Pat. 5,229,109) describe introducing two mutations, R38A and F42K, to obtain an IL-2 mutein with reduced ability to induce secretion of pro-inflammatory cytokines from natural killer (NK) cells. EP2639241B1 refers to IL-2 muteins that are at least 1,000 times less effective than native IL-2 in stimulating T_{reg} cells and refers to IL-2 muteins having the mutations selected from 1) R38K, F42I, Y45N, E62L, and E68V; 2) R38A, F42I, Y45N, E62L, and E68V; 3) R38K, F42K, Y45R, E62L, and E68V; or 4) R38A, F42A, Y45A, and E62A. U.S. Pat. Pub. 2014/0328791 refers to pegylated IL-2 with reduced affinity for CD25. Some IL-2 muteins have been conjugated to antibodies that target tumor antigens such as CEA, FAP, and PD-L1. See, e.g., Klein et al., Oncoimmunology 6(3):e1277306 (2017); Soerensen et al., J Clin Onc. 36: 15_suppl (2018); WO 2017/220989; and U.S. Pat. 9,206,260.

Interleukin-15 (IL-15) is a cytokine with structural similarity to IL-2. IL-15 binds to and signals through the IL-2Rβγ receptor and is secreted by mononuclear phagocytes and other immune cells following viral infection. IL-15 induces proliferation of NK and other cells of the innate immune system and is involved in killing of virally infected cells and cancer cells.

Unfortunately, the side effects of the current IL-2 and IL-15 drug candidates are significant, limiting the dosing amounts of the cytokines. In addition, the activation of T and other immune cells are not site specific. Further, there appears to be PK sinkers for IL-2 muteins even though their affinities for CD25 have been significantly reduced. Thus, there remains a need to develop improved cytokine therapeutics that are site selective when activating immune cells and have improved efficacy but reduced side effects.

### SUMMARY OF THE INVENTION

The present disclosure provides a prodrug comprising a cytokine moiety, a masking moiety, and a carrier moiety, wherein the masking moiety binds to the cytokine moiety and inhibits a biological activity of the cytokine moiety (e.g., prevents the cytokine moiety from binding to its receptor on a target cell, or reducing one or more biological activities of the cytokine moiety), the cytokine moiety is fused to the carrier moiety, and the masking moiety is fused to the cytokine moiety or to the carrier moiety through a cleavable peptide linker. In some embodiments, the masking moiety comprises an extracellular domain (ECD) of the receptor of the cytokine moiety.

In some embodiments, the cytokine moiety is a wildtype human cytokine or a mutein thereof, for example, a human IL-2 agonist polypeptide such as one comprising SEQ ID NO: 1 or an amino acid sequence that is at least 90% identical to SEQ ID NO: 1. In some embodiments, the human IL-2 agonist polypeptide comprises one or more mutations at position(s) selected from T3, K35, R38, F42, Y45, E62, E68, L72, A73, N88, C125, and Q126 (numbering according to SEQ ID NO: 1). In particular embodiments, the human IL-2 agonist polypeptide comprises an amino acid sequence selected from SEQ ID NOs: 8-17, 19-33, 36, 37, and 39-46.

In some embodiments, the masking moiety of the present prodrug comprises an ECD of human IL-2Rβ or a functional analog thereof. In further embodiments, the masking moiety comprises (i) two copies of the ECD of human IL-2Rβ or a functional analog thereof fused together through a peptide linker, or (ii) the ECD human IL-2Rβ or a functional analog thereof fused to an ECD of human IL-2Rγ or a functional analog thereof through a peptide linker. In some embodiments, the ECD of human IL-2Rγ or a functional analog thereof comprises SEQ ID NO: 6 or an amino acid sequence that is at least 90% identical to SEQ ID NO: 6. In particular embodiments, the ECD of human IL-2Rβ or a functional analog thereof comprises SEQ ID NO: 3, 4, or 5 or an amino acid sequence that is at least 90% to SEQ ID NO: 3, 4, or 5.

In some embodiments, the cytokine moiety of the present prodrug is a human IL-15 agonist polypeptide. The human IL-15 agonist polypeptide comprises SEQ ID NO: 2 or an amino acid sequence that is at least 90% identical to SEQ ID NO: 2. In some embodiments, the IL-15 agonist polypeptide comprises or further comprises (i) an IL-15Rα sushi domain comprising SEQ ID NO: 7 or (ii) an amino acid sequence that is at least 90% identical to SEQ ID NO: 7. In particular embodiments, the IL-15 masking domain comprises an ECD of human IL-2Rβ or a functional analog thereof or human IL-2Rγ or a functional analog thereof. In certain embodiments, the IL-15 masking domain comprises SEQ ID NO: 3, 4, 5, or 6, or an amino acid sequence that is at least 90% identical to SEQ ID NO: 3, 4, 5, or 6.

In some embodiments, the prodrug further comprises a second effector polypeptide, e.g., (i) a human IL-2 agonist polypeptide comprising a mutation at position 126 (numbering according to SEQ ID NO: 1), or (ii) a CCL19 polypeptide comprising an amino acid sequence that is at least 90% identical to SEQ ID NO: 123.

In some embodiments of the present prodrugs, the cytokine moiety is fused to the carrier moiety through a noncleavable peptide linker, such as one selected from SEQ ID NOs: 47-51.

In some embodiments of the present prodrugs, the cleavable peptide linker linking the masking moiety directly or indirectly (e.g., through the cytokine moiety) to the carrier moiety comprises a substrate sequence of urokinase-type plasminogen activator (uPA), matrix metallopeptidase (MMP) 2, or MMP9. In further embodiments, the cleavable peptide linker comprises substrate sequences of (i) both uPA and MMP2, (ii) both uPA and MMP9, or (iii) uPA, MMP2 and MMP9. In particular embodiments, the cleavable peptide linker comprises an amino acid sequence selected from SEQ ID NOs: 18, 34, 35, 38, 52-121, and 217. In certain embodiments, the cleavable peptide linker is cleavable by one or more proteases located at a tumor site or its surrounding environment, and the cleavage leads to activation of the prodrug at the tumor site or surrounding environment.

In some embodiments of the present prodrugs, the carrier moiety is a PEG molecule, an albumin (e.g., a human serum albumin) or a fragment thereof, an antibody Fc domain, or an antibody or an antigen-binding fragment thereof. In particular embodiments, the carrier moiety is an antibody Fc domain or an antibody comprises mutations L234A and L235A (“LALA”) (EU numbering). In particular embodiments, the carrier moiety is an antibody Fc domain or an antibody comprising knobs-into-holes mutations, and wherein the cytokine moiety and the masking moiety are fused to different polypeptide chains of the antibody Fc domain or to the different heavy chains of the antibody. In some embodiments, the cytokine moiety and the masking moiety are fused to the C-termini of the two different polypeptide chains of the Fc domain or to the C-termini of the two different heavy chains of the antibody. In other embodiments, the cytokine moiety and the masking moiety are fused to the N-termini of the two different polypeptide chains of the Fc domain or to the N-termini of the two different heavy chains of the antibody. In certain embodiments, the knobs-into-holes mutations comprise a T366Y “knob” mutation on a polypeptide chain of the Fc domain or a heavy chain of the antibody, and a Y407T “hole” mutation in the other polypeptide of the Fc domain or the other heavy chain of the antibody (EU numbering). In certain embodiments, the knobs-into-holes mutations comprise Y349C and/or T366W mutations in the CH3 domain of the “knob chain” and E356C, T366S, L368A, and/or Y407V mutations in the CH3 domain of the “hole chain” (EU numbering).

In particular embodiments, the carrier moiety is an antibody Fc domain comprising two polypeptide chains whose amino acid sequences respectively comprise an amino acid sequence selected from SEQ ID NOs: 195-198 and an amino acid sequence selected from SEQ ID NOs: 132-137 and 139.

In some embodiments, the carrier moiety is an antibody or an antigen-binding fragment thereof that specifically binds to one or more antigens selected from Guanyl cyclase C (GCC), carbohydrate antigen 19-9 (CA19-9), glycoprotein A33 (gpA33), mucin 1 (MUC1), carcinoembryonic antigen (CEA), insulin-like growth factor 1 receptor (IGF1-R), human epidermal growth factor receptor 2 (HER2), human epidermal growth factor receptor 3 (HER3), delta-like protein 3 (DLL3), delta-like protein 4 (DLL4), epidermal growth factor receptor (EGFR), glypican-3 (GPC3), c-MET, vascular endothelial growth factor receptor 1 (VEGFR1), vascular endothelial growth factor receptor 2 (VEGFR2), Nectin-4, Liv-1, glycoprotein NMB (GPNMB), prostate specific membrane antigen (PSMA), Trop-2, carbonic anhydrase IX (CA9), endothelin B receptor (ETBR), six transmembrane epithelial antigen of the prostate 1 (STEAP1), folate receptor alpha (FR-α), SLIT and NTRK-like protein 6 (SLITRK6), carbonic anhydrase VI (CA6), ectonucleotide pyrophosphatase/phosphodiesterase family member 3 (ENPP3), mesothelin, trophoblast glycoprotein (TPBG), CD19, CD20, CD22, CD33, CD40, CD56, CD66c, CD70, CD74, CD79b, CD98, CD123, CD138, CD352, CD47, signal-regulatory protein alpha (SIRPα), PD1, Claudin 18.2, Claudin 6, 5T4, BCMA, PD-L1, PD-1, Fibroblast Activation Protein alpha (FAPalpha), the Melanoma-associated Chondroitin Sulfate Proteoglycan (MCSP), and EPCAM.

In particular embodiments, the carrier moiety is an antibody comprising two heavy chains whose amino acid sequences respectively comprise SEQ ID NO: 209 and one of SEQ ID NOs: 210-215, and two light chains whose amino acid sequence comprises SEQ ID NO: 216. In certain embodiments, the carrier moiety is an antibody comprising two heavy chains whose amino acid sequences respectively comprise SEQ ID NO: 191 and one of SEQ ID NOs: 192, 193, and 206-208, and two light chains whose amino acid sequence comprises SEQ ID NO: 189.

In another aspect, the present disclosure provides an IL-2 mutein comprising a mutation at position A73, an IL-2 mutein comprising a K35N mutation, and an IL-2 mutein comprising an amino acid sequence selected from SEQ ID NO: 23-33, 36, 37, and 39-41. The novel IL-2 muteins may have significantly reduced binding to the trimeric IL-2 receptor.

In other aspects, the present disclosure provides also a pharmaceutical composition comprising a prodrug or IL-2 mutein of the present disclosure and a pharmaceutically acceptable excipient; a polynucleotide or polynucleotides encoding the prodrug or IL-2 mutein; an expression vector or vectors comprising the polynucleotide or polynucleotides; and a host cell comprising the vector(s), wherein the host cell may be a prokaryotic cell or an eukaryotic cell such as a mammalian cell. In some embodiments, the mammalian host cell has the gene or genes encoding uPA, MMP-2 and/or MMP-9 knocked out (e.g., containing null mutations of one or more of these genes). Accordingly, the present disclosure also provides a method of making the prodrug or IL-2 mutein, comprising culturing the host cell under conditions that allow expression of the prodrug or IL-2 mutein, wherein the host cell is a mammalian cell, and isolating the prodrug or IL-2 mutein.

The present disclosure also provides a method of treating a cancer or an infectious disease or stimulating the immune system in a patient (e.g., human patient) in need thereof, comprising administering to the patient a therapeutically effective amount of the prodrug, IL-2 mutein, or the pharmaceutical composition of the present disclosure. The patient may have, for example, a viral infection (e.g., HIV infection), or a cancer selected from the group consisting of breast cancer, lung cancer, pancreatic cancer, esophageal cancer, medullary thyroid cancer, ovarian cancer, uterine cancer, prostate cancer, testicular cancer, colorectal cancer, and stomach cancer. Also provided herein are a cytokine prodrug or IL-2 mutein for use in treating a cancer or an infectious disease or stimulating the immune system in the present method; use of a prodrug or IL-2 mutein for the manufacture of a medicament for treating a cancer or an infectious disease or stimulating the immune system in the present method; and articles of manufacture (e.g., kits) comprising one or more dosing units of the present prodrug or IL-2 mutein.

Other features, objects, and advantages of the invention are apparent in the detailed description that follows. It should be understood, however, that the detailed description, while indicating embodiments and aspects of the invention, is given by way of illustration only, not limitation. Various changes and modification within the scope of the invention will become apparent to those skilled in the art from the detailed description.

### BRIEF DESCRIPTIONS OF THE DRAWINGS

FIG. 1 shows the SDS-PAGE analysis of the mutant IL-2 polypeptides fused with carrier proteins.

FIGS. 2A-C show the results of CTLL2-based biological activity assay of IL-2 muteins fused to carrier proteins. FIG. 2A is a table listing the fusion proteins. FIG. 2B shows the results of the IL-2 mutein/Fc fusion proteins. FIG. 2C shows the results of the IL-2 mutein/human serum albumin (HSA) fusion proteins.

FIG. 3 is a schematic drawing illustrating an antibody-based IL-2 or IL-15 prodrug. An IL-2 or IL-15 agonist polypeptide is fused to the C-terminus of one of the heavy chains of the carrier antibody, optionally through a noncleavable peptide linker. An IL-2 or IL-15 antagonist polypeptide (masking moiety, e.g., an IL-2Rβ extracellular domain) is fused to the C-terminus of the other heavy chain of the carrier antibody through a cleavable peptide linker.

FIG. 4 shows the SDS-PAGE analysis of the 589A-IL-2 prodrugs expressed in HEK293 cells. 589A is a humanized antibody against Claudin 18.2 derived from rabbit B cell cloning. The prodrug samples were purified by Protein A affinity chromatography. The “activated” samples were the ones treated with protease. The SDS-PAGE was run under non-reducing condition.

FIG. 5 shows the SEC-HPLC analysis of the antibody 589A and the prodrug 589A-IL-2E.

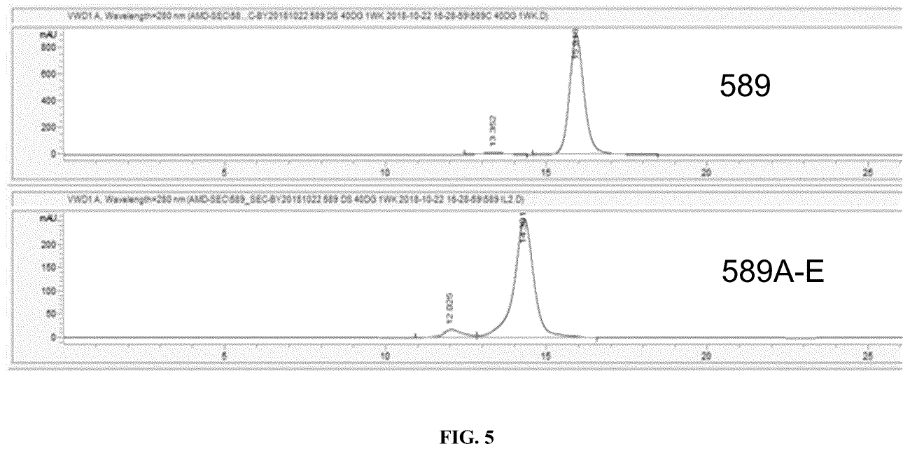

FIG. 6 shows the activation of 589A-IL-2 prodrugs using a CTLL2-based activity assay. JR1.55.1: 589A-IL-2E. JR1.55.2:589-IL-2F. MT: matriptase (a protease).

FIGS. 7A and B show the activation of the 589A-IL-2E (JR1.74.1) prodrug. FIG. 7A shows the binding of 589A-IL-2E and its activated version (+MT) to HEK293 cells expressing IL-2Rαβγ or IL-2Rβγ, as assayed by FACS analysis. MFI: mean fluorescent intensity. FIG. 7B shows the level of overall binding at 11 μg/ml of the prodrug.

FIG. 8 shows the activation of αPD-L1-IL-2B prodrugs using CTLL2-based activity assay. MT: matriptase.

FIG. 9 shows the analysis of antibody-dependent cellular cytotoxicity (ADCC) function of antibody 589A, 589A with enhanced ADCC functionality, and 589A with enhanced ADCC functionality fused to IL-2.

FIG. 10 shows the in vivo anti-cancer efficacy of prodrug 589A-IL-2E, as measured by tumor volumes in individual mice. Tecentriq®: anti-PD-L1 antibody atezolizumab.

FIG. 11 shows the in vivo anti-cancer efficacy of prodrug 589A-IL-2E, as measured by average tumor volume in each mouse group. It was the same study as shown in FIG. 10.

FIG. 12 shows the in vivo anti-cancer efficacy of prodrug 589A-IL-2E, as measured by survival. It was the same study as shown in FIG. 10.

### DETAILED DESCRIPTION OF THE INVENTION

As used herein and in the appended claims, the singular forms “a,” “or,” and “the” include plural referents unless the context clearly dictates otherwise. Reference to “about” a value or parameter herein includes (and describes) variations that are directed to that value or parameter per se. For example, description referring to “about X” includes description of “X.” Additionally, use of “about” preceding any series of numbers includes “about” each of the recited numbers in that series. For example, description referring to “about X, Y, or Z” is intended to describe “about X, about Y, or about Z.”

The term “antigen-binding moiety” refers to a polypeptide or a set of interacting polypeptides that specifically bind to an antigen, and includes, but is not limited to, an antibody (e.g., a monoclonal antibody, polyclonal antibody, a multi-specific antibody, a dual specific or bispecific antibody, an anti-idiotypic antibody, or a bifunctional hybrid antibody) or an antigen-binding fragment thereof (e.g., a Fab, a Fab′, a F(ab′)₂, a Fv, a disulfide linked Fv, a scFv, a single domain antibody (dAb), or a diabody), a single chain antibody, and an Fc-containing polypeptide such as an immunoadhesin. In some embodiments, the antibody may be of any heavy chain isotype (e.g., IgG, IgA, IgM, IgE, or IgD) or subtype (e.g., IgG₁, IgG₂, IgG₃, or IgG₄). In some embodiments, the antibody may be of any light chain isotype (e.g., kappa or lambda). The antibody may be human, non-human (e.g., from mouse, rat, rabbit, goat, or another non-human animal), chimeric (e.g., with a non-human variable region and a human constant region), or humanized (e.g., with non-human CDRs and human framework and constant regions). In some embodiments, the antibody is a derivatized antibody.

The term “cytokine agonist polypeptide” refers to a wildtype cytokine, or an analog thereof. An analog of a wildtype cytokine has the same biological specificity (e.g., binding to the same receptor(s) and activating the same target cells) as the wildtype cytokine, although the activity level of the analog may be different from that of the wildtype cytokine. The analog may be, for example, a mutein (i.e., mutated polypeptide) of the wildtype cytokine, and may comprise at least one, at least two, at least three, at least four, at least five, at least six, at least seven, at least eight, at least nine, or at least ten mutations relative to the wildtype cytokine.

The term “cytokine antagonist” or “cytokine mask” refers to a moiety (e.g., a polypeptide) that binds to a cytokine and thereby inhibiting the cytokine from binding to its receptor on the surface of a target cell and/or exerting its biological functions while being bound by the antagonist or mask. Examples of a cytokine antagonist or mask include, without limitations, a polypeptide derived from an extracellular domain of the cytokine's natural receptor that makes contact with the cytokine.

The term “effective amount” or “therapeutically effective amount” refers to an amount of a compound or composition sufficient to treat a specified disorder, condition, or disease, such as ameliorate, palliate, lessen, and/or delay one or more of its symptoms. In reference to a disease such as cancer, an effective amount may be an amount sufficient to delay cancer development or progression (e.g., decrease tumor growth rate, and/or delay or prevent tumor angiogenesis, metastasis, or infiltration of cancer cells into peripheral organs), reduce the number of epithelioid cells, cause cancer regression (e.g., shrink or eradicate a tumor), and/or prevent or delay cancer occurrence or recurrence. An effective amount can be administered in one or more administrations.

The term “functional analog” refers to a molecule that has the same biological specificity (e.g., binding to the same ligand) and/or activity (e.g., activating or inhibiting a target cell) as a reference molecule.

The term “fused” or “fusion” in reference to two polypeptide sequences refers to the joining of the two polypeptide sequences through a backbone peptide bond. Two polypeptides may be fused directly or through a peptide linker that is one or more amino acids long. A fusion polypeptide may be made by recombinant technology from a coding sequence containing the respective coding sequences for the two fusion partners, with or without a coding sequence for a peptide linker in between. In some embodiments, fusion encompasses chemical conjugation.

The term “pharmaceutically acceptable excipient” when used to refer to an ingredient in a composition means that the excipient is suitable for administration to a treatment subject, including a human subject, without undue deleterious side effects to the subject and without affecting the biological activity of the active pharmaceutical ingredient (API).

The term “subject” refers to a mammal and includes, but is not limited to, a human, a pet (e.g., a canine or a feline), a farm animal (e.g., cattle or horse), a rodent, or a primate.

As used herein, “treatment” or “treating” is an approach for obtaining beneficial or desired clinical results. Beneficial or desired clinical results include, but are not limited to, one or more of the following: alleviating one or more symptoms resulting from a disease, diminishing the extent of a disease, ameliorating a disease state, stabilizing a disease (e.g., preventing or delaying the worsening or progression of the disease), preventing or delaying the spread (e.g., metastasis) of a disease, preventing or delaying the recurrence of a disease, providing partial or total remission of a disease, decreasing the dose of one or more other medications required to treat a disease, increasing the patient's quality of life, and/or prolonging survival. The methods of the present disclosure contemplate any one or more of these aspects of treatment.

It is to be understood that one, some or all of the properties of the various embodiments described herein may be combined to form other embodiments of the present invention. The section headings used herein are for organizational purposes only and are not to be construed as limiting the subject matter described thereunder.

### Cytokine Prodrugs

The present disclosure provides cytokine prodrugs that are metabolized in vivo to become active cytokine therapeutics. The cytokine prodrugs have fewer side effects, better in vivo PK profiles (e.g., longer half-life) and better target specificity, and are more efficacious as compared to prior cytokine therapeutics. The present prodrugs comprise a cytokine agonist polypeptide (cytokine moiety) linked to a carrier moiety and masked (bound) by a cytokine antagonist (masking moiety). The cytokine antagonist, which may be, for example, an extracellular domain of a receptor for the cytokine, is linked to the cytokine moiety or to the carrier moiety through a cleavable linker (e.g., a cleavable peptide linker). The mask inhibits the cytokine moiety's biological functions while the mask is binding to it. The prodrugs may be activated at a target site (e.g., at a tumor site or the surrounding environment) in the patient by cleavage of the linker and the consequent release of the cytokine mask from the prodrug, exposing the previously masked cytokine moiety and allowing the cytokine moiety to bind to its receptor on a target cell and exert its biological functions on the target cell. In some embodiments, the carriers for the prodrugs are antigen-binding moieties, such as antibodies, that bind an antigen at the target site.

In some embodiments, the present prodrugs are pro-inflammatory cytokine prodrugs that are metabolized to become pro-inflammatory cytokines at a target site in the body targeted by the carrier. In further embodiments, the carrier in the prodrug is an antibody targeting a tumor antigen such that the prodrug is delivered to a tumor site in a patient and is metabolized locally (e.g., inside or in the vicinity of the tumor microenvironment) through cleavage of the linker linking the cytokine mask to the carrier or the cytokine moiety, making the pro-inflammatory cytokine moiety available to interact with its receptor on a target cell and stimulating the target immune cells locally.

While the description below exemplifies IL-2 and IL-15 prodrugs, prodrugs for other cytokines, in particular cytokines that are potent immune regulators and have strong side effects, are also contemplated in the present disclosure. These other cytokine prodrugs may be made according to the same principles as illustrated below for IL-2 and IL-15 prodrugs.

### A. Cytokine Moieties of the Prodrugs

In some embodiments, the present prodrugs comprise a pro-inflammatory cytokine agonist polypeptide, e.g., an IL-2 agonist polypeptide or an IL-15 agonist polypeptide.

### 1. IL-2 Agonist Polypeptides

An IL-2 prodrug may comprise an IL-2 agonist polypeptide (cytokine moiety), a carrier (carrier moiety), and an IL-2 antagonist (masking moiety), wherein the IL-2 agonist polypeptide is fused to the carrier directly or through a linker (e.g., cleavable or noncleavable peptide linker), and the IL-2 antagonist is linked to the IL-2 agonist polypeptide or to the carrier through a cleavable peptide linker. In the present IL-2 prodrugs, the IL-2 agonist polypeptide may be a wildtype IL-2 polypeptide such as a wildtype human IL-2 polypeptide (SEQ ID NO: 1), or an IL-2 mutein such as an IL-2 mutein derived from a human IL-2. The IL-2 mutein may have significantly reduced affinity for CD25 or the trimeric high-affinity IL-2R, as compared to wild type IL-2. In some embodiments, the IL-2 mutein has binding affinity for the high-affinity IL-2R that is 100 times, 300 times, 500 times, 1,000 times, or 10,000 times lower compared to wild type IL-2. Unless otherwise indicated, all residue numbers in IL-2 and IL-2 muteins described herein are in accordance with the numbering in SEQ ID NO: 1.

In one aspect, the present disclosure provides novel IL-2 muteins, which can be used as the IL-2 agonist polypeptides in the IL-2 prodrugs. The novel IL-2 mutein comprises a mutation at A73 (e.g., a mutation to T or another amino acid residue) and/or the K35N mutation. A73 has not been previously identified as one of the amino acid residues that interact with CD25. Thus, the present inventors were surprised that introduction of a mutation at this position (e.g., A73T) can lead to significantly reduced binding affinity of the IL-2 mutein for the trimeric IL-2 receptor, similar to that of the IL-2 mutein having mutations R38S/F42A/Y45A/E62A or the IL-2 mutein having mutations F42A/Y45A/L72G (see Example 1 below). Without being bound by theory, the present inventors contemplate that A73 and K35 are potential glycosylation sites on IL-2, and the mutation of these glycosylation sites modulates the IL-2 mutein's affinity for the IL-2Rs. The novel muteins will have safer clinical profiles and can be used in patients in need of IL-2 activity, such as patients in need of a stimulated immune system (e.g., cancer patients an AIDS patients). The novel IL-2 muteins can be used as a separate entity or in a conjugate (e.g., fused to a carrier such as in the present prodrugs).

In some embodiments, the novel IL-2 mutein of the present disclosure may comprise a mutation at A73 (e.g., A73T) and one or more mutations at position(s) selected from T3, D20, K35, R38, F42, F44, Y45, E62, E68, L72, N88, N90, C125, and Q126. In certain embodiments, the novel IL-2 mutein comprises mutations at R38, F42, Y45, and A73.

In some embodiments, the novel IL-2 mutein of the present invention may comprise the K35N mutation and one or more mutations one or more mutations at position(s) selected from T3, D20, R38, F42, F44, Y45, E62, E68, L72, A73, N88, N90, C125, and Q126. In certain embodiments, the novel IL-2 mutein comprises the mutation K35N and additional mutations at R38, F42, and Y45, with or without a mutation at A73.

In some embodiments, the IL-2 agonist polypeptide for the IL-2 prodrug may comprise one or more mutations at K35, R38, F42, F44, Y45, E62, E68, L72, and A73. In some embodiments, the IL-2 agonist polypeptide further comprises one or more mutations at D20, N88, N90, and Q126. Additional mutations at T3 and/or C125 may also be included. In particular embodiments, the IL-2 agonist polypeptide comprises an amino acid sequence selected from SEQ ID NOs: 8-17, 19-33, 36, 37, and 39-46.

In some embodiments, in order to ensure a base level of IL-2 agonistic activity, an IL-2 prodrug of the present invention may further comprise a second IL-2 agonist polypeptide that comprises mutations leading to significantly reduced affinity to the dimeric intermediate-affinity IL-2 receptor comparing to wild type IL-2. For example, the IL-2 muteins having mutations T3A/R38S/F42A/Y45A/E62A/C125S/Q126W (SEQ ID NOs: 30) with (SEQ ID NO: 130) or without (SEQ ID NO: 129) a linker show low but detectable levels of IL-2 activities (see Example 1 below).

### 2. IL-15 Agonist Polypeptides

In an IL-15 prodrug of the present disclosure, the IL-15 agonist polypeptide may be a wildtype IL-15 polypeptide such as a wildtype human IL-15 polypeptide (SEQ ID NO: 2), or an IL-15 mutein, such as an IL-15 mutein derived from a human wildtype IL-15, with reduced affinity for IL-15Rα or IL-2Rβ (CD122) compared to wild type IL-15.

### B. Masking Moieties of the Prodrugs

The cytokine antagonist, i.e., the masking moiety, in the present prodrug may comprise a peptide or an antibody or antibody fragment that binds to the cytokine moiety in the prodrug, masking the cytokine moiety and inhibiting its biological functions.

By way of example, IL-2 and IL-15 antagonists may comprise peptides and antibodies that bind IL-2 or IL-15 and interfere with the binding of the IL-2 or IL-15 moiety to its receptors, leading to the reduced biological activities of the IL-2 or IL-15 moiety while masked. In some embodiments, the IL-2 antagonist comprises an IL-2Rβ or IL-2Rγ extracellular domain or its functional analog such as one derived from human IL-2Rβ or IL-2Rγ (e.g., one of SEQ ID NOs: 3-6). In some embodiments, the IL-2 antagonist comprises a peptide identified from the screening of a peptide library. In some embodiments, the IL-2 antagonist comprises an antibody or fragment thereof that blocks the binding of IL-2 or IL-2 muteins to an IL-2 receptor. In particular embodiments, the IL-2 antagonist comprises a scFv, a Fab or a single chain Fab having the same CDR sequences as the antibody selected from hybridoma clones 4E12B2D10, 4E12B2, and 4E12, as disclosed in U.S. Pat. 4,411,993.

Human IL-2 binds to IL-2Rβ (CD122) with relatively low affinity (K_{D}˜3 μM, which is over 1,000 times weaker than the binding affinity of IL-2 for the intermediate affinity receptor IL-2Rβγ (Johnson et al., Eur Cytokine Netw. 5(1):23-34 (1994)). Thus, the present inventors were surprised that, when IL-2Rβ's extracellular domain (ECD) was fused to the same carrier molecule as an IL-2 mutein agonist polypeptide, the cell-based activity of the IL-2 mutein agonist polypeptide was significantly inhibited (see Example 4 below).

For an IL-15 prodrug, the masking moiety may be an extracellular domain of IL-2Rβ or IL-2Rγ or a functional analog thereof (e.g., one of SEQ ID NOs: 3-6).

### C. Carrier Moieties of the Prodrugs

The carrier moieties of the present prodrugs may be an antigen-binding moiety, or a moicty that is not an antigen-binding moiety. The carrier moiety may improve the PK profiles such as serum half-life of the cytokine agonist polypeptide, and may also target the cytokine agonist polypeptide to a target site in the body, such as a tumor site.

### 1. Antigen-Binding Carrier Moieties

The carrier moiety may be an antibody or an antigen-binding fragment thereof, or an immunoadhesin. In some embodiments, the antigen-binding moiety is a full-length antibody with two heavy chains and two light chains, a Fab fragment, a Fab' fragment, a F(ab′)₂ fragment, a Fv fragment, a disulfide linked Fv fragment, a single domain antibody, a nanobody, or a single-chain variable fragment (scFv). In some embodiments, the antigen-binding moiety is a bispecific antigen-binding moiety and can bind to two different antigens or two different epitopes on the same antigen. The antigen-binding moiety may provide additional and potentially synergetic therapeutic efficacy to the cytokine agonist polypeptide.

The cytokine (e.g., IL-2 or IL-15) agonist polypeptide and its mask may be fused to the N-terminus or C-terminus of the light chains and/or heavy chains of the antigen-binding moiety. By way of example, the cytokine (e.g., IL-2 or IL-15) agonist polypeptide and its mask may be fused to the antibody heavy chain or an antigen-binding fragment thereof or to the antibody light chain or an antigen-binding fragment thereof. In some embodiments, the cytokine (e.g., IL-2 or IL-15) agonist polypeptide is fused to the C-terminus of one or both of the heavy chains of an antibody, and the cytokine's mask is fused to the other terminus of the cytokine agonist polypeptide through a cleavable peptide linker. In some embodiments, the cytokine (c.g., IL-2 or IL-15) agonist polypeptide is fused to the C-terminus of one of the heavy chains of an antibody, and the cytokine's mask is fused to the C-terminus of the other heavy chain of the antibody through a cleavable peptide linker, wherein the two heavy chains contain mutations that allow the specific pairing of the two different heavy chains.

Strategies of forming heterodimers are well known (see, e.g., Spies et al., Mol Imm. 67(2)(A):95-106 (2015)). For example, the two heavy chain polypeptides in the prodrug may form stable heterodimers through “knobs-into-holes” mutations. “Knobs-into-holes” mutations are made to promote the formation of the heterodimers of the antibody heavy chains and are commonly used to make bispecific antibodies (see, e.g., U.S. Pat. 8,642,745). For example, the Fc domain of the antibody may comprise a T366W mutation in the CH3domain of the “knob chain” and T366S, L368A, and/or Y407V mutations in the CH3 domain of the “hole chain.” An additional interchain disulfide bridge between the CH3 domains can also be used, e.g., by introducing a Y349C mutation into the CH3 domain of the “knobs chain” and an E356C or S354C mutation into the CH3 domain of the “hole chain” (see, e.g., Merchant et al., Nature Biotech 16:677-81 (1998)). In other embodiments, the antibody moiety may comprise Y349C and/or T366W mutations in one of the two CH3 domains, and E356C, T366S, L368A, and/or Y407V mutations in the other CH3 domain. In certain embodiments, the antibody moiety may comprise Y349C and/or T366W mutations in one of the two CH3 domains, and S354C (or E356C), T366S, L368A, and/or Y407V mutations in the other CH3 domain, with the additional Y349C mutation in one CH3 domain and the additional E356C or S354C mutation in the other CH3 domain, forming an interchain disulfide bridge (numbering always according to EU index of Kabat; Kabat et al., “Sequences of Proteins of Immunological Interest,” 5th ed., Public Health Service, National Institutes of Health, Bethesda, Md. (1991)). Other knobs-into-holes technologies, such as those described in EP1870459A1, can be used alternatively or additionally. Thus, another example of knobs-into-holes mutations for an antibody moiety is having R409D/K370E mutations in the CH3 domain of the “knob chain” and D399K/E357K mutations in the CH3 domain of the “hole chain” (EU numbering).

In some embodiments, the antibody moiety in the prodrug comprises L234A and L235A (“LALA”) mutations in its Fc domain. The LALA mutations eliminate complement binding and fixation as well as Fcγ dependent ADCC (see, e.g., Hezareh et al. J. Virol. 75(24):12161-8 (2001)). In further embodiments, the LALA mutations are present in the antibody moiety in addition to the knobs-into-holes mutations.

In some embodiments, the antibody moiety comprises the M252Y/S254T/T256E (“YTE”) mutations in the Fc domain. The YTE mutations allow the simultaneous modulation of serum half-life, tissue distribution and activity of IgG₁ (see Dall' Acqua et al., J Biol Chem. 281: 23514-24 (2006); and Robbie et al., Antimicrob Agents Chemother. 57(12):6147-53 (2013)). In further embodiments, the YTE mutations are present in the antibody moiety in addition to the knobs-into-holes mutations. In particular embodiments, the antibody moiety has YTE, LALA and knobs-into-holes mutations or any combination thereof.

The antigen-binding moicty may bind to an antigen on the surface of a cell, such as an immune cell, for example, T cells, NK cells, and macrophages, or bind to a cytokine. For example, the antigen-binding moicty may bind to PD-1, LAG-3, TIM-3, TIGIT, CTLA-4, or TGF-beta and may be an antibody. The antibody may have the ability to activate the immune cell and enhance its anti-cancer activity.

The antigen-binding moiety may bind to an antigen on the surface of a tumor cell. For example, the antigen-binding moicty may bind to FAP alpha, 5T4, Trop-2, PD-L1, HER-2, EGFR, Claudin 18.2, DLL-3, GCP3, or carcinoembryonic antigen (CEA), and may be an antibody. The antibody may or may not have ADCC activity. The antibody may also be further conjugated to a cytotoxic drug.

In some embodiments, the antigen-binding moicty binds to guanyl cyclase C (GCC), carbohydrate antigen 19-9 (CA19-9), glycoprotein A33 (gpA33), mucin 1 (MUC1), insulin-like growth factor 1 receptor (IGF1-R), human epidermal growth factor receptor 2 (HER2), human epidermal growth factor receptor 3 (HER3), delta-like protein 3 (DLL3), delta-like protein 4 (DLL4), epidermal growth factor receptor (EGFR), glypican-3 (GPC3), c-MET, vascular endothelial growth factor receptor 1 (VEGFR1), vascular endothelial growth factor receptor 2 (VEGFR2), Nectin-4, Liv-1, glycoprotein NMB (GPNMB), prostatespecific membrane antigen (PSMA), Trop-2, carbonic anhydrase IX (CA9), endothelin B receptor (ETBR), six transmembrane epithelial antigen of the prostate 1 (STEAP1), folate receptor alpha (FR-α), SLIT and NTRK-like protein 6 (SLITRK6), carbonic anhydrase VI (CA6), ectonucleotide pyrophosphatase/phosphodiesterase family member 3 (ENPP3), mesothelin, trophoblast glycoprotein (TPBG), CD19, CD20, CD22, CD33, CD40, CD56, CD66c, CD70, CD74, CD79b, CD98, CD123, CD138, CD352, CD47, signal-regulatory protein alpha (SIRPα), Claudin 18.2, Claudin 6, BCMA, or EPCAM. In some embodiments, the antigen-binding moicty binds to an epidermal growth factor (EGF)-like domain of DLL3. In some embodiments, the antigen-binding moiety binds to a Delta/Serrate/Lag2 (DSL)-like domain of DLL3. In some embodiments, the antigen-binding moicty binds to an epitope located after the 374th amino acid of GPC3. In some embodiments, the antigen-binding moicty binds to a heparin sulfate glycan of GPC3. In some embodiments, the antigen-binding moiety binds to Claudin 18.2 and does not bind to Claudin 18.1. In some embodiments, the antigen-binding moicty binds to Claudin 18.1 with at least 10 times weaker binding affinity than to Claudin 18.2.

Exemplary antigen-binding moieties include trastuzumab, rituximab, brentuximab, cetuximab, panitumumab, GC33 (or a humanized version thereof), anti-EGFR antibody mAb806 (or a humanized version thereof), anti-dPNAG antibody F598, and antigen-binding fragments thereof. In some embodiments, the antigen-binding moicty has at least 90%, 91%, 92%, 93%, 94%, 95%, 96%, 97%, 98%, or 99% identity to trastuzumab, rituximab, brentuximab, cetuximab, or panitumumab, GC33 (or a humanized version thereof), anti-EGFR antibody mAb806 (or a humanized version thereof), anti-dPNAG antibody F598, or a fragment thereof. In some embodiments, the antigen-binding moiety has an antibody heavy chain with at least 90%, 91%, 92%, 93%, 94%, 95%, 96%, 97%, 98%, or 99% identity to the antibody heavy chain of trastuzumab, rituximab, brentuximab, cetuximab, panitumumab, GC33 (or a humanized version thereof), anti-EGFR antibody mAb806 (or a humanized version thereof), anti-dPNAG antibody F598, or a fragment thereof. In some embodiments, the antigen-binding moiety has an antibody light chain with at least 90%, 91%, 92%, 93%, 94%, 95%, 96%, 97%, 98%, or 99% identity to the antibody light chain of trastuzumab, rituximab, brentuximab, cetuximab, panitumumab, GC33 (or a humanized version thereof), anti-EGFR antibody mAb806 (or a humanized version thereof), anti-dPNAG antibody F598, or a fragment thereof. The antigen-binding moiety is fused to an IL-2 agonist polypeptide. In some embodiments, the antigen-binding moiety comprises the six complementarity determining regions (CDRs) of trastuzumab, rituximab, brentuximab, cetuximab, panitumumab, GC33, anti-EGFR antibody mAb806, or anti-dPNAG antibody F598.

A number of CDR delineations are known in the art and are encompassed herein. A person of skill in the art can readily determine a CDR for a given delineation based on the sequence of the heavy or light chain variable region. The “Kabat” CDRs are based on sequence variability and are the most commonly used (Kabat et al., Sequences of Proteins of Immunological Interest, 5th Ed. Public Health Service, National Institutes of Health, Bethesda, Md. (1991)). “Chothia” CDRs refer to the location of the structural loops (Chothia & Lesk, Canonical structures for the hypervariable regions of immunoglobulins, J. Mol. Biol., vol. 196, pp. 901-917 (1987)). The “AbM” CDRs represent a compromise between the Kabat CDRs and Chothia structural loops, and are used by Oxford Molecular's AbM antibody modeling software. The “Contact” CDRs are based on an analysis of the available complex crystal structures. The residues from each of these CDRs are noted below in Table 1, in reference to common antibody numbering schemes. Unless otherwise specified herein, amino acid numbers in antibodies refer to the Kabat numbering scheme as described in Kabat et al., supra, including when CDR delineations are made in reference to Kabat, Chothia, AbM, or Contact schemes. Using this numbering system, the actual linear amino acid sequence may contain fewer or additional amino acids corresponding to a shortening of, or insertion into, a framework region (FR) or CDR of the variable domain. For example, a heavy chain variable domain may include a single amino acid insert (residue 52a according to Kabat) after residue 52 of H2 and inserted residues (e.g., residues 82a, 82b, and 82c, etc. according to Kabat) after heavy chain FR residue 82. The Kabat numbering of residues may be determined for a given antibody by alignment at regions of homology of the sequence of the antibody with a “standard” Kabat numbered sequence.

TABLE 1
CDR Delineations According to Various Schemes

| CDR | Kabat | AbM | Chothia | Contact |
|---|---|---|---|---|
| VL-CDR1 | L24-L34 | L24-L34 | L26-L32 | L30-L36 |
| VL-CDR2 | L50-L56 | L50-L56 | L50-L52 | L46-L55 |
| VL-CDR3 | L89-L97 | L89-L97 | L91-L96 | L89-L96 |
| VH-CDR1 (Kabat nos.) | H31-H35B | H26-H35B | H26-H32 | H30-H35B |
| VH-CDR1 (Chothia nos.) | H31-H35 | H26-H35 | H26-H32 | H30-H35 |
| VH-CDR2 | H50-H65 | H50-H58 | H53-H55 | H47-H58 |
| VH-CDR3 | H95-H102 | H95-H102 | H95-H101 | H93-H101 |

In some embodiments, the CDRs are “extended CDRs,” and encompass a region that begins or terminates according to a different scheme. For example, an extended CDR can be as follows: L24-L36, L26-L34, or L26-L36 (VL-CDR1); L46-L52, L46-L56, or L50-L55 (VL-CDR2); L91-L97 (VL-CDR3); H47-H55, H47-H65, H50-H55, H53-H58, or H53-H65 (VH-CDR2); and/or H93-H102 (VH-CDR3).

In some embodiments, the antigen-binding moiety binds to HER2, and comprises a light chain having an amino acid sequence with at least 90%, 91%, 92%, 93%, 94%, 95%, 96%, 97%, 98%, or 99% identity to SEQ ID NO: 148, or a fragment thereof, and a heavy chain having an amino acid sequence with at least 90%, 91%, 92%, 93%, 94%, 95%, 96%, 97%, 98%, or 99% identity to SEQ ID NO: 149, or a fragment thereof. In some embodiments, the antigen-binding domain comprises CDR1, CDR2, and CDR3 from SEQ ID NO: 148, and CDR1, CDR2, and CDR3 from SEQ ID NO: 149.

In some embodiments, the antigen-binding moiety binds to CD20, and comprises a light chain having an amino acid sequence with at least 90%, 91%, 92%, 93%, 94%, 95%, 96%, 97%, 98%, or 99% identity to SEQ ID NO: 150, or a fragment thereof, and a heavy chain having an amino acid sequence with at least 90%, 91%, 92%, 93%, 94%, 95%, 96%, 97%, 98%, or 99% identity to SEQ ID NO: 151, or a fragment thereof. In some embodiments, the antigen-binding domain comprises CDR1, CDR2, and CDR3 from SEQ ID NO: 150, and CDR1, CDR2, and CDR3 from SEQ ID NO: 151.

In some embodiments, the antigen-binding moiety binds to CD30, and comprises a light chain having an amino acid sequence with at least 90%, 91%, 92%, 93%, 94%, 95%, 96%, 97%, 98%, or 99% identity to SEQ ID NO: 152, or a fragment thereof, and a heavy chain having an amino acid sequence with at least 90%, 91%, 92%, 93%, 94%, 95%, 96%, 97%, 98%, or 99% identity to SEQ ID NO: 153, or a fragment thereof. In some embodiments, the antigen-binding domain comprises CDR1, CDR2, and CDR3 from SEQ ID NO: 152, and CDR1, CDR2, and CDR3 from SEQ ID NO: 153.

In some embodiments, the antigen-binding moicty binds to EGFR, and comprises a light chain having an amino acid sequence with at least 90%, 91%, 92%, 93%, 94%, 95%, 96%, 97%, 98%, or 99% identity to SEQ ID NO: 154, or a fragment thereof, and a heavy chain having an amino acid sequence with at least 90%, 91%, 92%, 93%, 94%, 95%, 96%, 97%, 98%, or 99% identity to SEQ ID NO: 155, or a fragment thereof. In some embodiments, the antigen-binding domain comprises CDR1, CDR2, and CDR3 from SEQ ID NO: 154, and CDR1, CDR2, and CDR3 from SEQ ID NO: 155.

In some embodiments, the antigen-binding moicty binds to EGFR, and comprises a light chain having an amino acid sequence with at least 90%, 91%, 92%, 93%, 94%, 95%, 96%, 97%, 98%, or 99% identity to SEQ ID NO: 156, or a fragment thereof, and a heavy chain having an amino acid sequence with at least 90%, 91%, 92%, 93%, 94%, 95%, 96%, 97%, 98%, or 99% identity to SEQ ID NO: 157, or a fragment thereof. In some embodiments, the antigen-binding domain comprises CDR1, CDR2, and CDR3 from SEQ ID NO: 156, and CDR1, CDR2, and CDR3 from SEQ ID NO: 157.

In some embodiments, the antigen-binding moicty binds to c-MET, and comprises a light chain having an amino acid sequence with at least 90%, 91%, 92%, 93%, 94%, 95%, 96%, 97%, 98%, or 99% identity to SEQ ID NO: 158, or a fragment thereof, and a heavy chain having an amino acid sequence with at least 90%, 91%, 92%, 93%, 94%, 95%, 96%, 97%, 98%, or 99% identity to SEQ ID NO: 159, or a fragment thereof. In some embodiments, the antigen-binding domain comprises CDR1, CDR2, and CDR3 from SEQ ID NO: 158, and CDR1, CDR2, and CDR3 from SEQ ID NO: 159.

In some embodiments, the antigen-binding moiety binds to GPC3, and comprises a light chain having an amino acid sequence with at least 90%, 91%, 92%, 93%, 94%, 95%, 96%, 97%, 98%, or 99% identity to SEQ ID NO: 160, or a fragment thereof, and a heavy chain having an amino acid sequence with at least 90%, 91%, 92%, 93%, 94%, 95%, 96%, 97%, 98%, or 99% identity to SEQ ID NO: 161, or a fragment thereof. In some embodiments, the antigen-binding domain comprises CDR1, CDR2, and CDR3 from SEQ ID NO: 160, and CDR1, CDR2, and CDR3 from SEQ ID NO: 161.

In some embodiments, the antigen-binding moiety binds to Claudin 18.2, and comprises a light chain having an amino acid sequence with at least 90%, 91%, 92%, 93%, 94%, 95%, 96%, 97%, 98%, or 99% identity to SEQ ID NO: 162, or a fragment thereof, and a heavy chain having an amino acid sequence with at least 90%, 91%, 92%, 93%, 94%, 95%, 96%, 97%, 98%, or 99% identity to SEQ ID NO: 163, or a fragment thereof. In some embodiments, the antigen-binding domain comprises CDR1, CDR2, and CDR3 from SEQ ID NO: 162, and CDR1, CDR2, and CDR3 from SEQ ID NO: 163.

In some embodiments, the antigen-binding moiety binds to FAP alpha, and comprises a light chain having an amino acid sequence with at least 90%, 91%, 92%, 93%, 94%, 95%, 96%, 97%, 98%, or 99% identity to SEQ ID NO: 180 or 181, or a fragment thereof, and a heavy chain having an amino acid sequence with at least 90%, 91%, 92%, 93%, 94%, 95%, 96%, 97%, 98%, or 99% identity to SEQ ID NO: 182, or a fragment thereof. In some embodiments, the antigen-binding domain comprises CDR1, CDR2, and CDR3 from SEQ ID NO: 180 or 181, and CDR1, CDR2, and CDR3 from SEQ ID NO: 182. In some embodiments, the antigen-binding moiety binds to FAP alpha, and comprises a light chain variable domain having an amino acid sequence with at least 90%, 91%, 92%, 93%, 94%, 95%, 96%, 97%, 98%, or 99% identity to SEQ ID NO: 183, and a heavy chain variable domain having an amino acid sequence with at least 90%, 91%, 92%, 93%, 94%, 95%, 96%, 97%, 98%, or 99% identity to SEQ ID NO: 184. In some embodiments, the antigen-binding domain comprises CDR1, CDR2, and CDR3 from SEQ ID NO: 183, and CDR1, CDR2, and CDR3 from SEQ ID NO: 184. In particular embodiments, the humanized FAP antibody comprises a light chain amino acid sequence shown in SEQ ID NO: 180 or 181 and a heavy chain amino acid sequence shown in SEQ ID NO: 182.

In some embodiments, the antigen-binding moiety binds to carcinoembryonic antigen (CEA) and may be derived from antibody PRIA3 (U.S. Pat. 8,642,742). The anti-CEA antibody, and comprises a light chain having an amino acid sequence with at least 90%, 91%, 92%, 93%, 94%, 95%, 96%, 97%, 98%, or 99% identity to SEQ ID NO: 178, or a fragment thereof, and a heavy chain having an amino acid sequence with at least 90%, 91%, 92%, 93%, 94%, 95%, 96%, 97%, 98%, or 99% identity to SEQ ID NO: 179, or a fragment thereof. In some embodiments, the antigen-binding domain comprises CDR1, CDR2, and CDR3 from SEQ ID NO: 176, and CDR1, CDR2, and CDR3 from SEQ ID NO: 177. In certain embodiments, the PR1A3 antibody is a humanized antibody comprising a light chain variable domain amino acid sequence shown in SEQ ID NO: 178 and a heavy chain variable domain amino acid sequence shown in SEQ ID NO: 179.

In some embodiments, the antigen-binding moiety binds to PDL1, and comprises a light chain having an amino acid sequence with at least 90%, 91%, 92%, 93%, 94%, 95%, 96%, 97%, 98%, or 99% identity to SEQ ID NO: 189, or a fragment thereof, and a heavy chain having an amino acid sequence with at least 90%, 91%, 92%, 93%, 94%, 95%, 96%, 97%, 98%, or 99% identity to SEQ ID NO: 190, or a fragment thereof. In some embodiments, the antigen-binding domain comprises CDR1, CDR2, and CDR3 from SEQ ID NO: 189, and CDR1, CDR2, and CDR3 from SEQ ID NO: 190.

In some embodiments, the antigen-binding moiety binds to 5T4, and comprises a light chain variable domain having an amino acid sequence with at least 90%, 91%, 92%, 93%, 94%, 95%, 96%, 97%, 98%, or 99% identity to SEQ ID NO: 187 or 188, and a heavy chain variable domain having an amino acid sequence with at least 90%, 91%, 92%, 93%, 94%, 95%, 96%, 97%, 98%, or 99% identity to SEQ ID NO: 185 or 186, or a fragment thereof. In some embodiments, the antigen-binding domain comprises CDR1, CDR2, and CDR3 from SEQ ID NO: 187 or 188, and CDR1, CDR2, and CDR3 from SEQ ID NO: 185or 186.

In some embodiments, the antigen-binding moicty binds to Trop-2, and comprises a light chain variable region comprising a CDR1 comprising an amino acid sequence of KASQDVSIAVA (SEQ ID NO: 164), a CDR2 comprising an amino acid sequence of SASYRYT (SEQ ID NO: 165), and a CDR3 comprising an amino acid sequence of QQHYITPLT (SEQ ID NO: 166); and a heavy chain variable region comprising a CDR1 comprising an amino acid sequence of NYGMN (SEQ ID NO: 167), a CDR2 comprising an amino acid sequence of WINTYTGEPTYTDDFKG (SEQ ID NO: 168), and a CDR3comprising an amino acid sequence of GGFGSSYWYFDV (SEQ ID NO: 169).

In some embodiments, the antigen-binding moiety binds to mesothelin, and comprises light chain variable region comprising a CDR1 comprising an amino acid sequence of SASSSVSYMH (SEQ ID NO: 170), a CDR2 comprising an amino acid sequence of DTSKLAS (SEQ ID NO: 171), and a CDR3 comprising an amino acid sequence of QQWSGYPLT (SEQ ID NO: 172); and a heavy chain variable region comprising a CDR1 comprising an amino acid sequence of GYTMN (SEQ ID NO:173), a CDR2 comprising an amino acid sequence of LITPYNGASSYNQKFRG (SEQ ID NO: 174), and a CDR3 comprising an amino acid sequence of GGYDGRGFDY (SEQ ID NO: 175).

In some embodiments, the antigen-binding moicty comprises one, two or three antigen-binding domains. For example, the antigen-binding moicty is bispecific and binds to two different antigens selected from the group consisting of HER2, HER3, EGFR, 5T4, FAP alpha, Trop-2, GPC3, VEGFR2, Claudin 18.2 and PD-L1. In some embodiments, said bispecific antigen-binding moicty binds to two different epitopes of HER2.

### 2. Other Carrier Moieties

Other non-antigen-binding carrier moieties may be used for the present prodrugs. For example, an antibody Fc domain (e.g., a human IgG1, IgG2, IgG3, or IgG4 Fc), a polymer (e.g., PEG), an albumin (c.g., a human albumin) or a fragment thereof, or a nanoparticle can be used.

By way of example, the cytokine (e.g., IL-2 or IL-15) agonist polypeptide and its antagonist may be fused to an antibody Fc domain, forming an Fc fusion protein. In some embodiments, the cytokine (e.g., IL-2 or IL-15) agonist polypeptide is fused (directly or through a peptide linker) to the C-terminus or N-terminus of one of the Fc domain polypeptide chains, and the cytokine mask is fused to the C-terminus or N-terminus of the other Fc domain polypeptide chain through a cleavable peptide linker, wherein the two Fc domain polypeptide chains contain mutations that allow the specific pairing of the two different Fc chains. In some embodiments, the Fc domain comprises the holes-into-holes mutations described above. In further embodiments, the Fc domain may comprise also the YTE and/or LALA mutations described above.

The carrier moiety of the prodrug may comprise an albumin (e.g., human serum albumin) or a fragment thereof. An exemplary sequence of albumin is shown in SEQ ID NO: 124. In some embodiments, the albumin or albumin fragment is about 85% or more, about 90% or more, about 91% or more, about 92% or more, about 93% or more, about 94% or more, about 95% or more, about 96% or more, about 97% or more, about 98% or more, about 99% or more, about 99.5% or more, or about 99.8% or more identical to human serum albumin or a fragment thereof.

In some embodiments, the carrier moiety comprises an albumin fragment (e.g., a human serum albumin fragment) that is about 10 or more, 20 or more, 30 or more 40 or more, 50 or more, 60 or more, 70 or more, 80 or more, 90 or more, 100 or more, 120 or more, 140 or more, 160 or more, 180 or more, 200 or more, 250 or more, 300 or more, 350 or more, 400 or more, 450 or more, 500 or more, or 550 or more amino acids in length. In some embodiments, the albumin fragment is between about 10 amino acids and about 584 amino acids in length (such as between about 10 and about 20, about 20 and about 40, about 40 and about 80, about 80 and about 160, about 160 and about 250, about 250 and about 350, about 350 and about 450, or about 450 and about 550 amino acids in length). In some embodiments, the albumin fragment includes the Sudlow I domain or a fragment thereof, or the Sudlow II domain or the fragment thereof.

### D. Linker Components of the Prodrugs

The cytokine (e.g., IL-2 or IL-15) agonist polypeptide may be fused to the carrier moiety with or without a peptide linker. The peptide linker may be noncleavable. In some embodiments, the peptide linker is selected from SEQ ID NOs: 47-51. In particular embodiments, the peptide linker comprise the amino acid sequence GGGGSGGGGSGGGGS (SEQ ID NO: 49).

The cytokine (c.g., IL-2 or IL-15) mask may be fused to the cytokine moiety or to the carrier through a cleavable linker. The cleavable linker may contain one or more (e.g., two or three) cleavable moieties (CM). Each CM may be a substrate for an enzyme or protease selected from legumain, plasmin, TMPRSS-3/4, MMP-2, MMP-9, MT1-MMP, cathepsin, caspase, human neutrophil elastase, beta-secretase, uPA, and PSA. Examples of cleavable linkers include, without limitation, those comprising an amino acid sequence selected from SEQ ID NOs: 18, 34, 35, 38, 52-121, and 217.

Specific, nonlimiting examples of cytokine agonist polypeptides, cytokine masks, carriers, peptide linkers, and prodrugs are shown in the Sequences section below. Further, the prodrugs and novel IL-2 muteins of the present disclosure may be made by well known recombinant technology. For examples, one more expression vectors comprising the coding sequences for the polypeptide chains of the prodrugs may be transfected into mammalian host cells (c.g., CHO cells), and cells are cultured under conditions that allow the expression of the coding sequences and the assembly of the expressed polypeptides into the prodrug complex. In order for the prodrug to remain inactive, the host cells that express no or little uPA, MMP-2 and/or MMP-9 may be used. In some embodiments, the host cells may contain null mutations (knockout) of the genes for these proteases.

### Pharmaceutical Compositions

Pharmaceutical compositions comprising the prodrugs and muteins (i.e., the active pharmaceutical ingredient or API) of the present disclosure may be prepared by mixing the API having the desired degree of purity with one or more optional pharmaceutically acceptable excipients (see, e.g., Remington's Pharmaceutical Sciences, 16th Edition., Osol, A. Ed. (1980)) in the form of lyophilized formulations or aqueous solutions. Pharmaceutically acceptable excipients (or carriers) are generally nontoxic to recipients at the dosages and concentrations employed, and include, but are not limited to: buffers containing, for example, phosphate, citrate, succinate, histidine, acetate, or another inorganic or organic acid or salt thereof; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including sucrose, glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG).

Buffers are used to control the pH in a range which optimizes the therapeutic effectiveness, especially if stability is pH dependent. Buffers are preferably present at concentrations ranging from about 50 mM to about 250 mM. Suitable buffering agents for use with the present invention include both organic and inorganic acids and salts thereof, such as citrate, phosphate, succinate, tartrate, fumarate, gluconate, oxalate, lactate, and acetate. Additionally, buffers may comprise histidine and trimethylamine salts such as Tris.

Preservatives are added to retard microbial growth, and are typically present in a range from 0.2%-1.0% (w/v). Suitable preservatives for use with the present invention include octadecyldimethylbenzyl ammonium chloride; hexamethonium chloride; benzalkonium halides (e.g., chloride, bromide, iodide), benzethonium chloride; thimerosal, phenol, butyl or benzyl alcohol; alkyl parabens such as methyl or propyl paraben; catechol; resorcinol; cyclohexanol, 3-pentanol, and m-cresol.

Tonicity agents, sometimes known as “stabilizers” are present to adjust or maintain the tonicity of liquid in a composition. When used with large, charged biomolecules such as proteins and antibodies, they are often termed “stabilizers” because they can interact with the charged groups of the amino acid side chains, thereby lessening the potential for inter- and intra-molecular interactions. Tonicity agents can be present in any amount between 0.1% to 25% by weight, or more preferably between 1% to 5% by weight, taking into account the relative amounts of the other ingredients. Preferred tonicity agents include polyhydric sugar alcohols, preferably trihydric or higher sugar alcohols, such as glycerin, erythritol, arabitol, xylitol, sorbitol and mannitol.

Non-ionic surfactants or detergents (also known as “wetting agents”) are present to help solubilize the therapeutic agent as well as to protect the therapeutic protein against agitation-induced aggregation, which also permits the formulation to be exposed to shear surface stress without causing denaturation of the active therapeutic protein or antibody. Non-ionic surfactants are present in a range of about 0.05 mg/ml to about 1.0 mg/ml, preferably about 0.07 mg/ml to about 0.2 mg/ml.

Suitable non-ionic surfactants include polysorbates (20, 40, 60, 65, 80, etc.), polyoxamers (184, 188, etc.), PLURONIC® polyols, TRITON®, polyoxyethylene sorbitan monoethers (TWEEN®-20, TWEEN®-80, etc.), lauromacrogol 400, polyoxyl 40 stearate, polyoxyethylene hydrogenated castor oil 10, 50 and 60, glycerol monostearate, sucrose fatty acid ester, methyl cellulose and carboxymethyl cellulose. Anionic detergents that can be used include sodium lauryl sulfate, dioctyle sodium sulfosuccinate and dioctyl sodium sulfonate. Cationic detergents include benzalkonium chloride or benzethonium chloride.

The choice of pharmaceutical carrier, excipient or diluent may be selected with regard to the intended route of administration and standard pharmaceutical practice. Pharmaceutical compositions may additionally comprise any suitable binder(s), lubricant(s), suspending agent(s), coating agent(s) or solubilizing agent(s).

There may be different composition/formulation requirements dependent on the different delivery systems. By way of example, pharmaceutical compositions useful in the present invention may be formulated to be administered using a mini-pump or by a mucosal route, for example, as a nasal spray or acrosol for inhalation or ingestible solution, or parenterally in which the composition is formulated by an injectable form, for delivery, by, for example, an intravenous, intramuscular or subcutaneous route.

In some embodiments, the pharmaceutical composition of the present disclosure is a lyophilized protein formulation. In other embodiments, the pharmaceutical composition may be an aqueous liquid formulation.

### Methods of Treatment

The cytokine (e.g., IL-2 or IL-15) prodrug can be used to treat a disease, depending on the antigen bound by the antigen-binding domain. In some embodiments, the IL-2 or IL-15 prodrug is used to treat cancer. In some embodiments, the IL-2 or IL-15 prodrug is used to treat an infection, for example when the drug molecule is an antibacterial agent or an antiviral agent.

In some embodiments, a method of treating a disease (such as cancer, a viral infection, or a bacterial infection) in a subject comprises administering to the subject an effective amount of an IL-2 or IL-15 prodrug.

In some embodiments, the cancer is a solid cancer. In some embodiments, the cancer is a blood cancer or a solid tumor. Exemplary cancers that may be treated include, but are not limited to, leukemia, lymphoma, kidney cancer, bladder cancer, urinary tract cancer, cervical cancer, brain cancer, head and neck cancer, skin cancer, uterine cancer, testicular cancer, esophageal cancer, liver cancer, colorectal cancer, stomach cancer, squamous cell carcinoma, prostate cancer, pancreatic cancer, lung cancer such as nonsmall cell lung cancer, cholangiocarcinoma, breast cancer, and ovarian cancer.

In some embodiments, the cytokine (e.g., IL-2 or IL-15) prodrug is used to treat a bacterial infection such as sepsis. In some embodiments, the bacteria causing the bacterial infection is a drug-resistant bacteria. In some embodiments, the antigen-binding moiety binds to a bacterial antigen.

In some embodiments, the cytokine (c.g., IL-2 or IL-15) prodrug is used to treat a viral infection. In some embodiments, the virus causing the viral infection is hepatitis C (HCV), hepatitis B (HBV), human immunodeficiency virus (HIV), a human papilloma virus (HPV). In some embodiments, the antigen-binding moicty binds to a viral antigen.

Generally, dosages and routes of administration of the present pharmaceutical compositions are determined according to the size and conditions of the subject, according to standard pharmaceutical practice. In some embodiments, the pharmaceutical composition is administered to a subject through any route, including orally, transdermally, by inhalation, intravenously, intra-arterially, intramuscularly, direct application to a wound site, application to a surgical site, intraperitoneally, by suppository, subcutaneously, intradermally, transcutaneously, by nebulization, intrapleurally, intraventricularly, intra-articularly, intraocularly, intracranially, or intraspinally. In some embodiments, the composition is administered to a subject intravenously.

In some embodiments, the dosage of the pharmaceutical composition is a single dose or a repeated dose. In some embodiments, the doses are given to a subject once per day, twice per day, three times per day, or four or more times per day. In some embodiments, about 1 or more (such as about 2, 3, 4, 5, 6, or 7 or more) doses are given in a week. In some embodiments, the pharmaceutical composition is administered weekly, once every 2 weeks, once every 3 weeks, once every 4 weeks, weekly for two weeks out of 3 weeks, or weekly for 3 weeks out of 4 weeks. In some embodiments, multiple doses are given over the course of days, weeks, months, or years. In some embodiments, a course of treatment is about 1 or more doses (such as about 2, 3, 4, 5, 7, 10, 15, or 20 or more doses).

Unless otherwise defined herein, scientific and technical terms used in connection with the present disclosure shall have the meanings that are commonly understood by those of ordinary skill in the art. Exemplary methods and materials are described below, although methods and materials similar or equivalent to those described herein can also be used in the practice or testing of the present disclosure. In case of conflict, the present specification, including definitions, will control. Generally, nomenclature used in connection with, and techniques of, cell and tissue culture, molecular biology, immunology, microbiology, genetics, analytical chemistry, synthetic organic chemistry, medicinal and pharmaceutical chemistry, and protein and nucleic acid chemistry and hybridization described herein are those well-known and commonly used in the art. Enzymatic reactions and purification techniques are performed according to manufacturer's specifications, as commonly accomplished in the art or as described herein. Further, unless otherwise required by context, singular terms shall include pluralities and plural terms shall include the singular. Throughout this specification and embodiments, the words “have” and “comprise,” or variations such as “has,” “having,” “comprises,” or “comprising,” will be understood to imply the inclusion of a stated integer or group of integers but not the exclusion of any other integer or group of integers. It is understood that aspects and variations of the invention described herein include “consisting” and/or “consisting essentially of” aspects and variations. All publications and other references mentioned herein are incorporated by reference in their entirety. Although a number of documents are cited herein, this citation does not constitute an admission that any of these documents forms part of the common general knowledge in the art.

### Exemplary Embodiments

Further particular embodiments of the present disclosure are described as follows. These embodiments are intended to illustrate the compositions and methods described in the present disclosure and are not intended to limit the scope of the present disclosure.
1. An isolated mutant interleukin-2 (IL-2) polypeptide, wherein said mutant IL-2 polypeptide comprises the amino acid sequence of SEQ ID NO: 1 with up to seven amino acid substitutions, wherein one of the mutations is at position 73.
2. An isolated mutant interleukin-2 (IL-2) polypeptide, wherein said mutant IL-2 polypeptide comprises the amino acid sequence of SEQ ID NO: 1 with up to seven amino acid substitutions, wherein one of the mutations is K35N.
3. An isolated mutant interleukin-2 (IL-2) polypeptide, wherein said mutant IL-2 polypeptide comprises the amino acid sequence of SEQ ID NO: 1 with up to seven amino acid substitutions, wherein one of the mutations is A73T.
4. The mutant interleukin-2 polypeptide of any one of embodiments 1-3, further comprising additional amino acid mutations, wherein said mutations are at positions corresponding to residues 42 and 45 of human IL-2.
5. The mutant interleukin-2 polypeptide of any one of embodiments 1-3, further comprising additional amino acid mutations, wherein said mutations are at positions corresponding to residues 38, 42 and 45 of human IL-2.
6. The mutant interleukin-2 polypeptide of any one of embodiments 1-3, further comprising additional amino acid mutations, wherein said mutations are at positions corresponding to residues 38, 42, 45 and 62 of human IL-2.
7. The mutant interleukin-2 polypeptide of any one of embodiments 4-6, wherein mutation at position 42 is selected from the group of F42A, F42G, F421, F42S, F42T, F42Q, F42E, F42N, F42D, F42R, and F42K.
8. The mutant interleukin-2 polypeptide of any one of embodiments 4-6, wherein mutation at position 45 is selected from the group of Y45A, Y45G, Y45S, Y45T, Y45Q, Y45E, Y45N, Y45D, Y45R, and Y45K.
9. The mutant interleukin-2 polypeptide of any one of embodiments 5 and 6, wherein mutation at position 38 is selected from the group of R38A, R38K, and R38S.
10. The mutant interleukin-2 polypeptide of embodiment 6, wherein mutation at position 62 is selected from the group of E62L, E62A, and E62I.
11. The mutant interleukin-2 polypeptide of any one of embodiments 1 to 10, further comprising mutations T3A and C125S
12. A chimeric molecule, which comprises the mutant interleukin-2 polypeptide of any one of embodiments 1 to 11 and a carrier, wherein said mutant IL-2 polypeptide is operationally linked to said carrier, wherein said carrier is selected from a PEG molecule, an albumin molecule, an albumin fragment, an IgG Fc, and an antigen binding molecule.
13. Chimeric molecule of embodiment 12, wherein said carrier is an antigen binding molecule, and wherein said antigen binding molecule is an antibody or an antibody fragment.
14. Chimeric molecule of embodiment 12, wherein said carrier is an antigen binding molecule, and wherein said antigen binding molecule is a bispecific antibody.
15. Chimeric molecule of any of embodiments 13 and 14, wherein said antigen is selected from the group of PD-L1, PD-1, Fibroblast Activation Protein alpha (FAPalpha), CEA, BCMA, CD20, Trop-2, HER2, 5T4, the Melanoma-associated Chondroitin Sulfate Proteoglycan (MCSP), PSMA, EGFR, and Claudin 18.2.
16. A prodrug of a cytokine (e.g. IL-2 or IL-15), comprising a cytokine (e.g., IL-2 or IL-15) mutein, a masking moiety or an antagonist of the cytokine (e.g., IL-2 or IL-15) and a cleavable peptide linker, such as a prodrug of IL-2, which comprises an IL-2 agonist polypeptide (A), a masking moiety (MM), and at least one cleavable peptide linker; wherein said masking moiety comprises the IL-2 receptor beta subunit extracellular domain or a functional analog thereof.
17. Prodrug of embodiment 16, wherein said IL-2 antagonist or masking moiety (MM) comprises the extracellular domain of the IL-2 receptor beta subunit, which comprises an amino acid sequence at least 85%, at least 90%, or at least 95% identical to SEQ ID NO: 3.
18. Prodrug of embodiment 16, wherein said IL-2 antagonist or masking moiety comprises the extracellular domain of the IL-2 receptor beta subunit, which comprises an amino acid sequence of SEQ ID NO: 3.
19. Prodrug of any of embodiments 16-18, wherein said IL-2 agonist polypeptide (A) comprises an amino acid sequence at least 90% identical to SEQ ID NO: 1, or said IL-15 agonist polypeptide (A) comprises an amino acid sequence at least 90% identical to SEQ ID NO: 2.
20. Prodrug of any of embodiments 16-18, wherein said IL-2 agonist polypeptide (A) comprises an analog of human IL-2 containing one or more mutations at position or positions selected from T3, K35, R38, F42, Y45, E62, E68, L72, A73, and C125; and wherein said mutations are referred to according to the numbering of the human IL-2 with the amino acid sequence of SEQ ID NO: 1.
21. Prodrug of any of embodiments 16-18, wherein said IL-2 agonist polypeptide (A) is the mutant IL-2 selected from any one of embodiments 1-11.
22. Prodrug of any of embodiments 16-18, wherein said IL-2 agonist polypeptide comprises an amino acid sequence selected from SEQ ID NOs: 8-17, 19-33, 36, 37, and 39-46.
23. Prodrug of any of embodiments 16-22, wherein it further comprises a carrier (C), wherein said carrier is selected from a PEG molecule, an albumin, an albumin fragment, an Fc, and an antigen-binding molecule.
24. A prodrug of IL-2 (or IL-15), wherein its IL-2 (or IL-15) activity is activated at the site of a tumor or its surrounding area, comprising: an agonist polypeptide of IL-2 (or IL-15) operationally fused or conjugated to a carrier, an antagonist of IL-2 (or IL-15) which inhibits or impacts the binding of above said IL-2 (or IL-15) agonist polypeptide to its receptor; and a cleavable peptide linker which links the IL-2 (or IL-15) antagonist to said IL-2 (or IL-15) agonist polypeptide or its carrier; wherein said cleavable peptide linker is cleavable by a protease or proteases found inside a tumor or its surrounding environment; and wherein said carrier is selected from a protein, an antibody, or a polyethene glycol (PEG) polymer.
25. Prodrug of embodiment 24, wherein said IL-2 or IL-15 antagonist comprises the extracellular domain of the IL-2 receptor beta subunit or a functional analog thereof.
26. Prodrug of any of embodiments 24 and 25, wherein said IL-2 or IL-15 antagonist or masking moiety (MM) comprises the extracellular domain of the IL-2 receptor beta subunit, which comprises an amino acid sequence at least 85%, at least 90%, or at least 95% identical to SEQ ID NO: 3.
27. Prodrug of any of embodiments 24 and 25, wherein said IL-2 or IL-15 antagonist or masking moiety is the extracellular domain of the IL-2 receptor beta subunit, which comprises an amino acid sequence of SEQ ID NO: 3.
28. Prodrug of any of embodiments 24-27, wherein said IL-2 or IL-15 antagonist or masking moiety further comprises the IL-2 receptor gamma subunit or a functional equivalent thereof.
29. Prodrug of any of embodiments 24-27, wherein said IL-2 or IL-15 antagonist or masking moiety further comprises a second IL-2 receptor beta subunit or a functional equivalent thereof.
30. Prodrug of any of embodiments 24-29, wherein said IL-2 agonist polypeptide (A) has an amino acid sequence at least 90% identical to SEQ ID NO: 1.
31. Prodrug of any of embodiments 24-29, wherein said IL-2 agonist polypeptide (A) is an analog of human IL-2 containing one or more mutations at position or positions selected from T3, K35, R38, F42, Y45, E62, E68, L72, A73, and C125 (e.g., a mutation at A73 and the K35N mutation); and wherein said mutations are referred to according to the numbering of the human IL-2 with amino acid sequence of SEQ ID NO: 1.
32. Prodrug of any of embodiments 24-29, wherein said IL-2 agonist polypeptide (A) is the mutant IL-2 selected from any one of embodiments 1-11.
33. Prodrug of any one of embodiments 24-29, wherein said IL-2 agonist polypeptide comprises an amino acid sequence selected from SEQ ID NOs: 8-17, 19-33, 36, 37, and 39-46.
34. A Prodrug of IL-15 which comprises an IL-15 agonist polypeptide (A), a masking moiety (MM), a carrier (C), and at least one cleavable peptide linker; wherein said IL-15 agonist polypeptide (A) comprises an amino acid sequence at least 90%, at least 95%, or 100% identical to that of SEQ ID NO: 2; said masking moiety (MM) is selected from the IL-2 receptor beta subunit extracellular domain, a functional analog of said IL-2 receptor beta subunit extracellular domain IL-2 receptor beta subunit extracellular domain, an IL-2 receptor beta subunit extracellular domain fused to IL-2 receptor gamma subunit extracellular domain through a peptide linker, and a dimer of IL-2 receptor beta subunit extracellular domains linked to each other through a cleavable peptide linker; and said carrier is selected from an albumin, an albumin fragment, a Fc, and an antigen binding molecule.
35. Prodrug of embodiment 34, wherein said prodrug of IL-15 also comprises a Sushi domain of the IL-15 receptor alpha subunit; and wherein said Sushi domain comprises an amino acid sequence at least 95% or 100% identical to SEQ ID NO: 7.
36. Prodrug of any one of embodiments 34 and 35, wherein said IL-2 receptor beta subunit extracellular domain comprises an amino acid sequence at least 95% or 100% identical to SEQ ID NO: 3.
37. Prodrug of any one f embodiments 28 and 34, wherein said gamma subunit extracellular domain comprises an amino acid sequence at least 95% or 100% identical to SEQ ID NO: 6.
38. Prodrug of any one of embodiments 23-37, wherein said carrier (C) is an antigen binding molecule; wherein said antigen binding molecule is an antibody comprising two heavy chains and two light chains.
39. Prodrug of embodiment 38, wherein said cytokine (e.g., IL-2 or IL-15) agonist polypeptide is fused to the C-terminus of one of the heavy chains of said antibody, optionally through a peptide linker, and said cytokine (e.g., IL-2 or IL-15) antagonist or masking moiety (MM) is fused to the C-terminus of the second heavy chain through a cleavable peptide linker; and wherein the two heavy chain-fusion proteins form a heterodimer through “knobs-into-holes” mutations.
40. Prodrug of embodiment 38, wherein said cytokine (e.g., IL-2 or IL-15) agonist polypeptide is fused to the N-terminus of one of the heavy chains of said antibody, optionally through a peptide linker, and said cytokine (e.g., IL-2 or IL-15) antagonist or masking moiety (MM) is fused to the N-terminus of the second heavy chain through a cleavable peptide linker; and wherein the two heavy chain-fusion proteins form a heterodimer through “knobs-into-holes” mutations.
41. Prodrug of embodiment 38, wherein the cytokine (e.g., IL-2 or IL-15) agonist polypeptide is fused or conjugated to the N-terminus of one or both of the heavy chains of said antibody or antibody fragment, directly or through a peptide linker, and said cytokine (e.g., IL-2 or IL-15) antagonist or masking moiety (MM) is fused to the N-terminus of the light chain through a cleavable peptide linker, forming a heavy chain-fusion polypeptide and a light chain-fusion polypeptide.
42. Prodrug of any of embodiments 23-41, wherein said carrier is an antigen binding molecule, wherein said antigen binding molecule binds to one or more antigens selected from Guanyl cyclase C (GCC), carbohydrate antigen 19-9 (CA19-9), glycoprotein A33 (gpA33), mucin 1 (MUC1), carcinoembryonic antigen (CEA), insulin-like growth factor 1 receptor (IGF1-R), human epidermal growth factor receptor 2 (HER2), human epidermal growth factor receptor 3 (HER3), delta-like protein 3 (DLL3), delta-like protein 4 (DLL4), epidermal growth factor receptor (EGFR), glypican-3 (GPC3), c-MET, vascular endothelial growth factor receptor 1 (VEGFR1), vascular endothelial growth factor receptor 2 (VEGFR2), Nectin-4, Liv-1, glycoprotein NMB (GPNMB), prostatespecific membrane antigen (PSMA), Trop-2, carbonic anhydrase IX (CA9), endothelin B receptor (ETBR), six transmembrane epithelial antigen of the prostate 1 (STEAP1), folate receptor alpha (FR-α), SLIT and NTRK-like protein 6 (SLITRK6), carbonic anhydrase VI (CA6), ectonucleotide pyrophosphatase/phosphodiesterase family member 3 (ENPP3), mesothelin, trophoblast glycoprotein (TPBG), CD19, CD20, CD22, CD33, CD40, CD56, CD66e, CD70, CD74,CD79b, CD98, CD123, CD138, CD352, CD47, signal-regulatory protein alpha (SIRPα), PD1, Claudin 18.2, Claudin 6, FAP-alpha., 5T4, BCMA, PD-L1, PD-1 and EPCAM.
43. Prodrug of any one of embodiments 23-42, wherein it further comprises another effector polypeptide.
44. Prodrug of any one of embodiments 23-42, wherein it further comprises another effector polypeptide, wherein said effector polypeptide is another IL-2 mutein comprising an amino acid mutation at position 126.
45. Prodrug of any one of embodiments 23-42, wherein it further comprises another effector polypeptide, wherein said effector polypeptide is a CCL19 polypeptide comprising an amino acid sequence at least 95% identical to SEQ ID NO: 123.
46. Prodrug of any one of embodiments 16-45, wherein said cleavable peptide linker is cleavable by a protease or proteases found at a tumor site or its surrounding environment.
47. Prodrug of any one of embodiments 16-45, wherein said prodrug is activatable at the site of a tumor.
48. Prodrug of any one of embodiments 16-45, wherein said cleavable peptide linker comprises a substrate of uPA.
49. Prodrug of any one of embodiments 16-45, wherein said cleavable peptide linker comprises a substrate of MMP2 and/or MMP9.
50. Prodrug of any one of embodiments 16-45, wherein said cleavable peptide linker comprises substrates of both uPA and MMP2, both uPA and MMP9, or uPA, MMP2 and MMP9.
51. Prodrug of any one of embodiments 16-45, wherein said cleavable peptide linker contains an enzyme substrate amino acid sequence selected from LSGRSDNH (SEQ ID NO: 52), ISSGLLSS (SEQ ID NO: 53), and GPLGVR (SEQ ID NO: 54).
52. Prodrug of any one of embodiments 16-45, wherein said cleavable peptide linker contains both enzyme substrate amino acid sequences LSGRSDNH (SEQ ID NO: 52) and ISSGLLSS (SEQ ID NO: 53).
53. Prodrug of any one of embodiments 16-45, wherein said cleavable peptide linker contains both enzyme substrate amino acid sequences LSGRSDNH (SEQ ID NO: 52) and GPLGVR (SEQ ID NO: 54); or ISSGLLSS (SEQ ID NO: 53) and GPLGVR (SEQ ID NO: 54).
54. Prodrug of any one of embodiments 16-45, wherein said cleavable peptide linker comprises an amino acid sequence selected from SEQ ID NOs: 55-78.
55. A polynucleotide which encodes the mutant IL-2 of any one of embodiments 1-11.
56. A polynucleotide or polynucleotides which encode the chimeric molecule of any one of embodiments 12-15, or the prodrug of any one of embodiments 16-54.
57. An expression vector or vectors comprising the polynucleotide or polynucleotides of embodiment 55 or 56.
58. A host cell transfected with the vector of embodiment 57.
59. Host cell of embodiment 58, wherein said host cell has the gene or genes encoding uPA, MMP-2 and/or MMP-9 knocked out.
60. A method of producing said mutant IL-2 of any one of embodiments 1-11, said chimeric molecule of any of embodiments 12-15, or said prodrug of any of embodiments 16-54, comprising culturing the host cell of embodiment 58 or 59.
61. A pharmaceutical composition comprising as active ingredient the mutant IL-2 of any one embodiments 1-11 or the prodrug of any one of embodiments 16-54.
62. A pharmaceutical composition comprising as active ingredient the chimeric molecule of any one of embodiments 12-15.
63. A method of treating breast, lung, pancreatic, esophageal, medullary thyroid, ovarian, uterine, prostatic, testicular, colon, rectal or stomach cancer, or infectious disease in a human subject in need thereof, comprising administering to the human subject said pharmaceutical composition of embodiment 61 or 62.

In order that this invention may be better understood, the following examples are set forth. These examples are for purposes of illustration only and are not to be construed as limiting the scope of the invention in any manner.

### EXAMPLES

The materials and methods used in the studies described in Examples 1-6 are described below.

### Transient Transfection of HEK293 Cells

Expression plasmids were co-transfected into 3×10⁶ cell/ml freestyle HEK293 cells at 2.5-3 μg/ml using PEI (polyethylenimine). For Fc-based IL-2 prodrugs, the ratios for the Fc-IL-2 mutein fusion polypeptide and the Fc-masking moiety fusion polypeptide were in a 1:2 ratio. For antibody-based IL-2 prodrugs, ratios for the knob heavy chain (containing IL-2 agonist polypeptide) and hole heavy chain (containing the masking moiety) and the light chain DNA were in a 2:1:2 molar ratio. The cell cultures were harvested 6 days after transfection by centrifuging at 9,000 rpm for 45 min followed by 0.22 μM filtration.

### Protein Purification

The purifications of the proteins of the antibody-based IL-2 prodrugs were carried out by using three steps of chromatography, including: 1) Protein A affinity chromatography; 2) Q Sepharose Fast Flow and 3) Capto MMC ImpRes. Q FF was equilibrated by the buffer containing 25 mM Tris and 100 mM NaCl (pH 7.5). Capto MMC ImpRes was equilibrated using the buffer A (20 mM phosphate, 30 mM NaCl, pH 6.2) and eluted using a 10 CV linear gradient with buffer B (20 mM phosphate, 0.5 M Arginine, pH 6.2).

### SEC-HPLC Analysis

SEC-HPLC was carried out using an Agilent 1100 Series of HPLC system with a TSKgel G3000SWXL column (7.8 mmIDX 30 cm, 5 μm particle size) ordered from Tosoh Bioscience. A sample of up to 100 μl was loaded. The column was run with a buffer containing 200 mM K₃PO₄, 250 mM KCl, pH 6.5. The flow rate was 0.5 ml/min. The column was run at room temperature. The protein elution was monitored both at 220 nm and 280 nm.

### SDS-PAGE Analysis

10 μl of the culture supernatants or 20 μg of purified protein samples were mixed with Bolt™ LDS Sample Buffer (Novex) with or without reduce reagents. The samples were heated at 70° C. for 3 min and then loaded to a NuPAGE™ 4-12% BisTris Gel (Invitrogen). The gel was run in NuPAGE™ MOPS SDS Running buffer (Invitrogen) at 200 Volts for 40 min and then stained with Coomassie.

### Proteolytic Treatment

The proteases, human u-Plasminogen Activator (uPA)/Urokinase (R&D systems or human Matriptase/ST14 (R&D systems) were added to the precursor molecules at 81 nM and 250 nM, respectively, and incubated at 37° C. overnight.

### CTLL2 Assay

CTLL2 cells were grown in the RPMI 1640 medium supplemented with L-glutamine, 10% fetal bovine serum, 10% non-essential amino acids, 10% sodium pyruvate, and 55 μM beta-mercaptoethanol. CTLL2 cells were non-adherent and maintained at 5×10⁴-1×10⁶ cells/ml in medium with 100 ng/ml of IL-2. Generally, cells were split twice per week. For bioassays, it was best to use cells no less than 48 hours after passage.

Samples were diluted at 2× concentration in 50 μl/well in a 96 well plate. The IL-2standards were titrated from 20 ng/ml (2× concentration) to 3× serial dilutions for 12 wells. Samples were titer tested as appropriate. CTLL2 cells were washed 5 times to remove IL-2, dispensed 5000 cells/well in 50 μl and cultured overnight or at least 18 hours with the samples. Subsequently, 100 μl/well Cell Titer Glo reagents (Promega) were added and luminescence were measured.

### Enzyme-linked Immunosorbent Assay (ELISA)

10 μg/ml of IL-2 proteins in PBS were seeded to the 96-well plate at 100 μl/well and coated at 4 degree for overnight. The wells were washed by PBS three times and blocked with 100 μl 2% milk/PBS for 1 hr. Then the wells were washed three times by PBS and 100 μl protein samples with 3-fold serial dilution were added to the wells for 1 hr incubation at room temperature. After three times of PBS washing, 100 μl of HRP conjugated anti-IgG antibody were added and incubated at RT for 1 hr. Subsequently, the wells were washed again by PBS for 3 times, detection reagents were added and OD450 nM were measured.

### FACS Analysis

Stable HEK 293 cell lines expressing IL-2Rαβγ or IL-2Rβγ were cultured. The cells were detached with non-enzymatic cell dissociation solutions. Cells were counted and the cell density was adjusted to approximately 3 million cells/ml with FACS washing buffer, which comprised 3% FBS in PBS. 50 μl cells (150,000 cells) were added into each well of a 96 well plate. Primary antibody or supernatant expressing the antibody of interest was added to the cells at prespecified concentration. The plate was incubated on ice for 1 hr. The plate was washed 3 times with the FACS washing buffer. Fluorescence conjugated secondary antibody was added to the cells (concentration depending on manufacture instruction). The plate was incubated on ice for 1 hr. The plate was washed again. PI staining solution was added at 0.1 μg/ml and the plate was incubated for 10 min on ice. The cell fluorescence was measured with flow cytometry instrument.

### Antibody-Dependent Cellular Cytotoxicity (ADCC)

Claudin 18.2 antibody, Claudin 18.2 antibody with ADCC enhanced and Claudin 18.2 antibody-IL-2 samples were analyzed for their capability to induce ADCC against HEK293 cells stably expressing human CLD18.2 or human CLD18.1.

To enrich human peripheral blood mononuclear cells, human blood from healthy donors was diluted twice in phosphate buffer (PBS) and blood cells were layered on Ficoll (Lymphocyte Separation Medium 1077 g/ml, PAA Laboratories, cat. no. J15-004). Peripheral blood mononuclear cells (MNCs) were collected from the interphase, washed and resuspended in RPMI 1640 culture medium supplemented with 10% heat-inactivated fetal calf scrum, 2 mM L-glutamine.

To set up ADCC assays, target cells were labeled with a fluorescence enhancing ligand (BADTA, Perkin Elmer cytotoxicity assay kit DELFIA EuTDA Cytotoxicity Reagents, cat. no. AD0116) for 30 minutes. After extensive washing in RPMI-10 supplemented with 10 mM probenecid (Sigma, cat. no. P8761), 10-20 mM HEPES, and 10% heat-inactivated fetal calf serum, the cells were adjusted to 1×10⁵ cells/ml. Labeled target cells, effector cells (MNCs), and supernatants containing monoclonal antibodies adjusted to a concentration of 10 μg/ml were added to round-bottom microtiter plates. For isolated effector cells, an effector to target (E:T) ratio of 100:1 (data not shown for 50:1 and 25:1) was used. After incubation for 2 hr at 37° C., assays were stopped by centrifugation, and fluorescence ligand release from duplicates was measured in europium counts in a time-resolved fluorometer. Percentage of cellular cytotoxicity was calculated using the following formula: % specific lysis (experimental release counts—spontaneous release counts)/(maximal release counts×spontaneous release counts)×100, with maximal fluorescence ligand release determined by adding Triton X-100 (0.25% final concentration) to target cells, and spontaneous release measured in the absence of antibodies and effector cells.

### In vivo Efficacy Study with a Syngeneic Tumor Model

Six-week old Balb/c mice (Taconic Biosciences) were injected subcutaneously with 1×10⁶ CT26/18.2 cells. After 7 days, tumors were measured using digital calipers and tumor volume was calculated (V=(ab2)p/6, where b is the shorter of 2 dimensions measured). Mice were then randomized into treatment groups such that all groups had approximately the same mean tumor size (127.6 mm³). Mice were then treated with placebo or test article at 10 mg/Kg in 100 μl via intraperitoneal injection. Dosing was performed on days 7, 9, 11, 13, 15 and 18. Tumors were measured every 2-3 days, and mice were sacrificed when tumors reached 2000 mm³.

### Example 1: Expression and Testing of Mutant IL-2 Agonist Polypeptides

The human IL-2 (SEQ ID NO: 1) is a polypeptide of 133 amino acids. A number of mutant human IL-2 agonist polypeptides were expressed as part of a fusion molecule and tested for their biological activities (Table 2). The pairing polypeptide, if any, is also shown.

TABLE 2
Selected Mutant IL-2 Agonist Polypeptide Fusions

| | Pairing | | | |
|---|---|---|---|---|
| | Poly- | | | |
| Fusion | peptide | | | Linker |
| SEQ | SEQ | Il-2 Mutations | Carrier | SEQ |
| 126 | — | T3A/C125S/F42A/Y45A/L72G | N-HSA | 49 |
| 127 | — | T3A/C125S/R38S/F42A/Y45A/E62A | N-HSA | 49 |
| 130 | — | T3A/C125S/R38S/F42A/Y45A/E62A/Q126W | N-HSA | 49 |
| 129 | — | T3A/C125S/R38S/F42A/Y45A/E62A/Q126W | N-HSA | No Linker |
| 132 | 194 | T3A/C125S/R38S/F42A/Y45A/E62A | N-Fc | 49 |
| 133 | 194 | T3A/C125S/R38S/F42R/Y45K/E62A | N-Fc | 49 |
| 135 | 194 | T3A/C125S/R38S/F42A/Y45A/A73T | N-Fc | 49 |
| 136 | 194 | T3A/C125S/K35N/R38S/F42A/Y45A/A73T | N-Fc | 49 |
| 137 | 194 | T3A/C125S/R38S/F42A/Y45A/E62A | N-Fc | No linker |
| 139 | 194 | T3A/C125S/F42A/Y45A/E62A/N88E | N-Fc | 49 |

SEQ: SEQ ID NO. HSA: human serum albumin. N-HSA: the carrier HSA is located at the N-terminus of the IL-2 polypeptide; N-Fc: the carrier Fc is located at the N-terminus of the IL-2 polypeptide. C-Fc: the carrier Fc is located at the C-terminus of the IL-2 polypeptide.

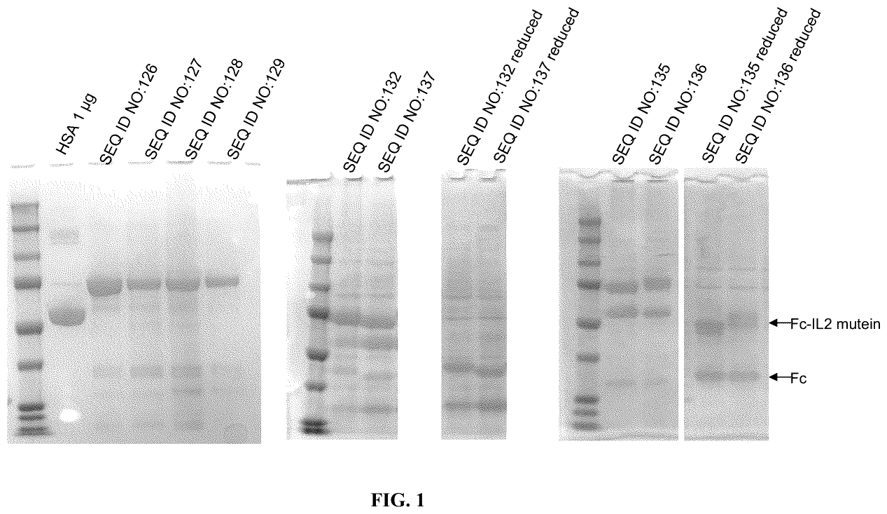

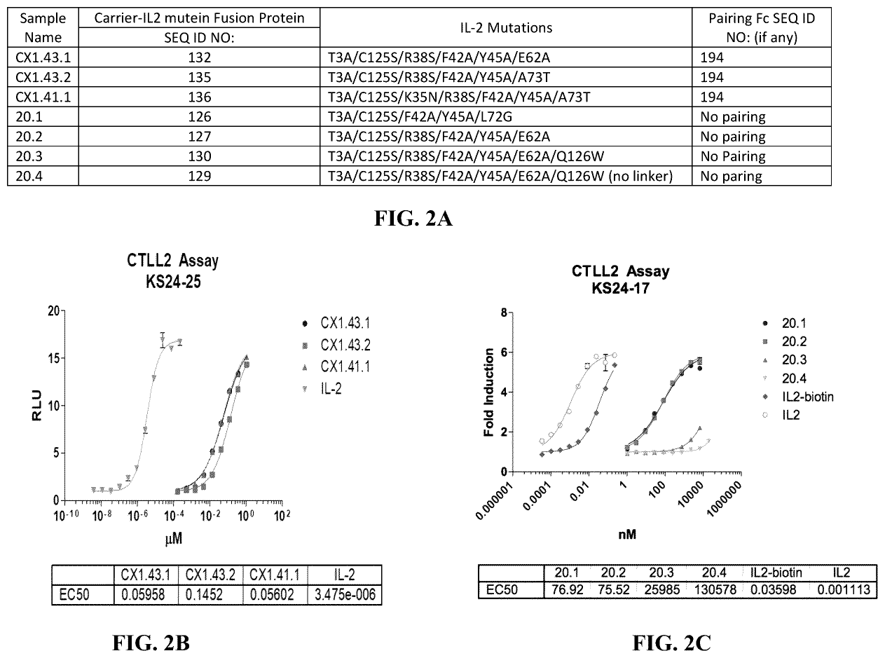

The expressed IL-2 polypeptides were tested by SDS-PAGE (FIG. 1). Their biological activities were tested using the CTLL2 cell-based activity assay described above. As shown in FIG. 2B, the Fc fusion proteins with IL-2 muteins T3A/C125S/R38S/F42A/Y45A/A73T (SEQ ID NO: 135) and T3A/C125S/R38S/F42A/Y45A/E62A (SEQ ID NO: 132) displayed similar activities in the cell-based assay, with an EC₅₀ of approximately 60 nM, while the Fc fusion protein with IL-2 mutein T3A/C125S/K35N/R38S/F42A/Y45A/A73T (SEQ ID NO: 136) showed a lower activity with an EC₅₀ of about 140 nM. The Fc fusion protein with IL-2 mutein T3A/C125S/R38S/F42R/Y45K/E62A (SEQ ID NO: 133) showed an EC₅₀ of about 87 nM (data not shown). The Fc fusion protein with IL-2 mutein T3A/C125S/R38S/F42A/Y45A/E62A (SEQ ID NO: 137), which had no peptide linker between the IL-2 mutein and the Fc, showed an EC₅₀ of about 72 nM (data not shown). FIG. 2C showed that the activities of the human albumin fusion protein with IL-2 muteins T3A/C125S/F42A/Y45A/L72G (SEQ ID NO: 126) and T3A/C125S/R38S/F42A/Y45A/E62A (SEQ ID NO: 127) had similar cell-based activities with an EC₅₀ of approximately 77 nM and 76 nM, respectively.

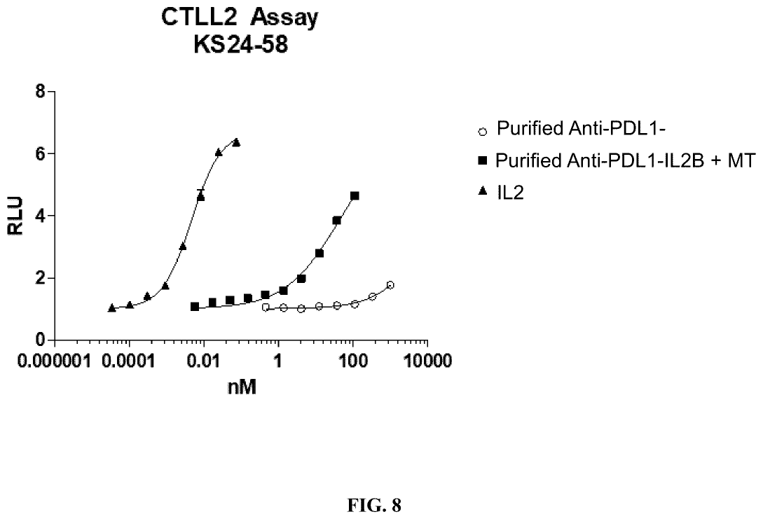

These results show that the introduction of the additional mutation A73T to IL-2 mutein had similar effects on the IL-2 activities as the mutation of E62A. In general, the IL-muteins shown in SEQ ID NO: 135, 132, 126 and 127 had similar cell-based activities, which were significantly lower than the activity of wild type IL-2. This was presumably due to the significantly reduced or abolished binding of the muteins to IL-2Rα. The IL-2 mutein with mutations T3A/C125S/R38S/F42A/Y45A/E62A (SEQ ID NO: 132) had significantly reduced binding affinity for IL-2Rα, as shown in FIG. 8 (also see below). In addition, the introduction of the additional two mutations of A73T and K35N further reduced the activities of the IL-2 mutein.

We also noted that additional mutations at position 126 led to further significantly reduced levels of the cell-based activities, although they still possessed some IL-2 activities, as shown in IL-2 muteins T3A/C125S/R38S/F42A/Y45A/E62A/Q126W (SEQ ID NO: 130) and T3A/C125S/R38S/F42A/Y45A/E62A/Q126W (no linker) (SEQ ID NO: 129) (FIG. 2C).

### Example 2: Design of IL-2 Antagonists or Masking Moieties

In order to construct a prodrug platform for IL-2, we designed various IL-2 antagonists (masks) using the human IL-2 Receptor beta subunit and gamma subunit. Exemplary mask designs are listed in Table 3, which include:
1) a single copy of the IL-2Rβ subunit extracellular domain, which was fused to the C-terminus of a Fc fragment through a cleavable peptide linker

(GGGGSGGGGSGGGGSLSGRSDNHGGGGS; SEQ ID NO: 18)

containing one protease substrate peptide (SEQ ID NO: 195);
2) one copy of the IL-2Rβ subunit extracellular domain fused with one copy of the IL-2Rgamma subunit extracellular domain, which were fused into the C-terminus of a Fc through a cleavable peptide linker

(GGGGSGGGGSGGGGSISSGLLSSGGSGGSLSGRSDNHGGGGS; SEQ
ID NO: 38)

containing two protease cleavage sites (SEQ ID NO: 196);
3) A single of the IL-2Rβ subunit extracellular domain, which was fused to the C-terminus of a Fc through the cleavable linker SEQ ID NO: 38 (SEQ ID NO: 197);
4) two copies of the IL-2Rβ subunit extracellular domain linked with each other, which were fused to the C-terminus of a Fc through the cleavable linker SEQ ID NO: 38 (SEQ ID NO: 198), wherein underlines indicate protease substrate sequences.

TABLE 3
Designs of IL-2 Antagonists or Masks

| | | Cleavable |
|---|---|---|
| Fc-Mask Fusion | | Peptide Linker |
| SEQ | Mask Design | SEQ |
| 195 | IL-2Rβ | 18 |
| 196 | IL-2Rβ and γ | 30 |
| 197 | IL-2Rβ | 30 |
| 198 | IL-2Rβ and β | 30 |

SEQ: SEQ ID NO.

The Fc fragment used in these mask polypeptides contained the “hole” mutation Y407T. The IL-2 muteins were fused to the Fc fragment that contained the “knob” mutation T366Y.

The designs of the IL-2 prodrugs are shown in Table 4. Each said prodrug comprised an IL-2 agonist polypeptide fused to the C-terminus of a Fc (SEQ ID NO: 132, 133 or 136) and was co-expressed with one of the Fc-mask fusion polypeptide (SEQ ID NO: 195, 196, 197, or 198).

TABLE 4
Designs of IL-2 Prodrugs

| Prodrug | Fc-Mask | Fc-IL-2 Mutein | Folds of activation after |
|---|---|---|---|
| design | Fusion SEQ | Fusion SEQ | protease cleavage |
| 1 | 195 | 132 | 16.1 |
| 2 | 196 | 132 | — |
| 3 | 197 | 132 | 22.0 |
| 4 | 198 | 132 | 0.5 |
| 5 | 198 | 133 | 10.4 |
| 6 | 198 | 132 | 1.4 |
| 7 | 198 | 136 | 6.8 |

The prodrugs were treated with proteases, human u-Plasminogen Activator (uPA)/Urokinase or human Matriptase/ST14. The data show that protease treatment resulted in a 0.5 to 22 folds of activation of IL-2 functions in the CTLL2 assay (Table 4). The results showed that both the IL-2Rβ extracellular domain and the dimer of the IL-2Rβ extracellular domain worked as a mask for the IL-2 agonist polypeptide. The cleavable peptide linkers with one or two cleavable sites both worked. We noted that the mask comprising both the IL-2Rβ and γ subunits extracellular domain did not express well.

### Example 3: Optimization of the Masking Moieties

In order to discover improved versions of the IL-2 antagonist or mask with higher folds of activations upon protease cleavage, a number of mutations in the IL-2Rβ extracellular domain were constructed. The constructs were expressed as homodimer in HEK293 cells and their binding affinities with IL-2 as measured by the ELISA method described above are shown in Table 5. IL-2Rβ extracellular domain with single mutation R15Y (SEQ ID NO:199), V75Q (SEQ ID NO: 202) or V75F (SEQ ID NO: 203) completely lost the binding affinities to IL-2 in ELISA assay. IL-2Rβ extracellular domain with single mutation S69H (SEQ ID NO: 201) or E136Q (SEQ ID NO: 204) lost the binding activities to IL-2 at pH 7.4, but displayed 2-fold better binding affinities for IL-2 at pH 6.4 (Table 5). IL-2Rβ extracellular domain with double mutations E136Q/H138R (SEQ ID NO: 205) displayed similar binding affinity to IL-2 at pH 7.4 as that of the wild type, though its binding affinities to IL-2 at pH 6.4 was enhanced by two folds (Table 5). IL-2Rβ extracellular domain with mutation D68E (SEQ ID NO: 200) displayed 2-fold increased binding affinities to IL-2 at both pH 7.4 and pH 6.4 (Table 5).

TABLE 5
Designs of IL-2RB Mutations and Their binding Affinities for IL-2

| Fc-Mask Fusion | | KD at pH 7.4 | KD at pH 6.4 |
|---|---|---|---|
| SEQ | Mutations | (μg/ml) | (μg/ml) |
| 195 | WT | 219 | 268.7 |
| 199 | R15Y | — | — |
| 200 | D68E | 123.7 | 117.7 |
| 201 | S69H | — | 186.6 |
| 202 | V75Q | — | — |
| 203 | V75F | — | — |
| 204 | E136Q | — | 127.6 |
| 205 | E136Q/H138R | 268.7 | 104.8 |

“—”: No or minimal binding

### Example 4: IL-2 Prodrugs with Antibody Molecules as Carriers

Fusing a cytokine polypeptide to an antibody allows targeted delivery of the cytokine to a disease site. However, there will be significant competition for binding to the cytokine receptor if there are high affinity cytokine receptors on the immune cells, which can be abundant in immune organs. In this study, IL-2 muteins with significantly reduced binding affinity for IL-2Rα were fused to antibody carriers. This kind of antibody IL-2 prodrug can be activated at the disease sites targeted by the antibody and can have significantly improved PK profiles and enhanced disease site specificity.

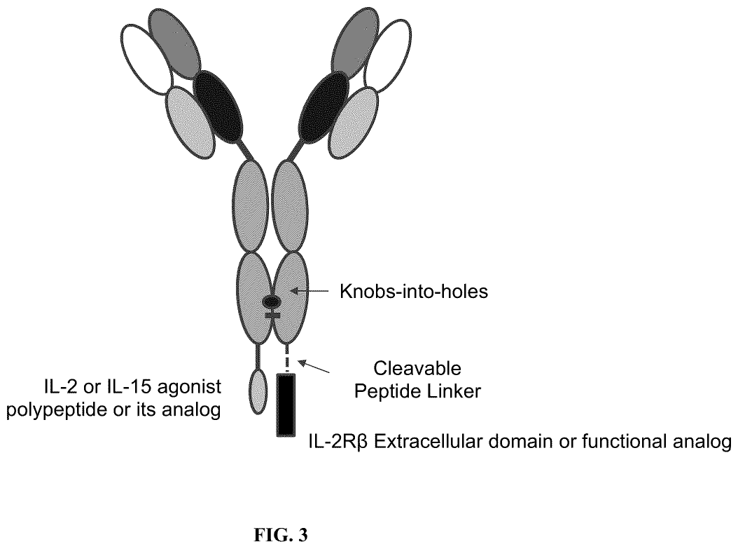

An antibody against Claudin 18.2 (589A sequences) and an antibody against PD-L1 (atezolizumab) were used as examples to demonstrate the feasibility of the novel IL-2 prodrug platform. The structure of the antibody-based prodrug is illustrated in FIG. 3. The different combination designs of the 589A-IL-2-mask fusion molecules are listed in Table 6.

TABLE 6
Designs of 589A-IL-2 Prodrugs

| | Knob | Hole | | | |
|---|---|---|---|---|---|
| | HC-IL2 | HC-mask | LC | Linker | |
| Construct | SEQ | SEQ | SEQ | SEQ | Mask |
| 589A-IL-2A | 209 | 210 | 216 | 217 | WT |
| 589A-IL-2B | 209 | 211 | 216 | 49 | WT |
| 589A-IL-2C | 209 | 212 | 216 | 61 | WT |
| 589A-IL-2D | 209 | 213 | 216 | 62 | WT |
| 589A-IL-2E | 209 | 214 | 216 | 63 | WT |
| 589A-IL-2F | 209 | 215 | 216 | 63 | D68E |

HC: heavy chain.
LC: light chain.
SEQ: SEQ ID NO.

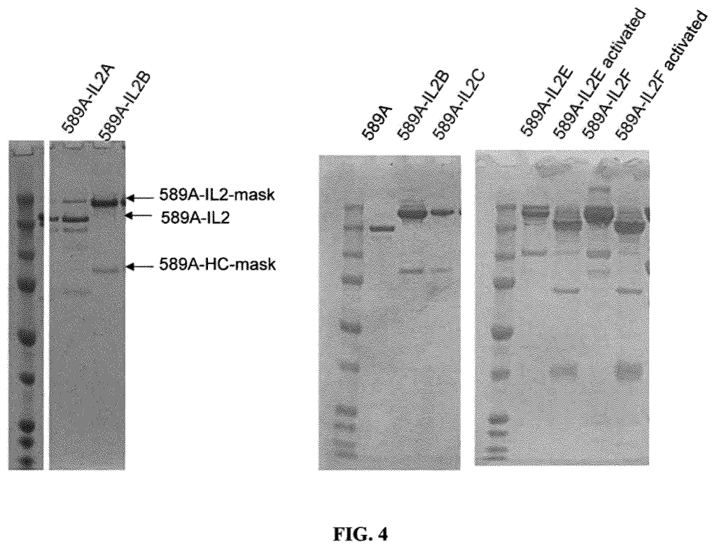

More than 80% of 589A-IL-2A molecules were cleaved without protease treatment, potentially due to the presence of proteases in the cells or secreted by the cells during cell culture (FIG. 4). 589A-IL-2B, which has a non-cleavable linker [(GGGGS)₃] (SEQ ID NO: 49), showed stable assembly of the heterotetramer antibody and displayed no stimulatory activities in the CTLL2 Assay (FIG. 4, Table 7). This data indicates the effectiveness of the masking moiety as an antagonist for IL-2. 589A-IL-2C molecules were assembled correctly and showed a 38-fold inhibition on the IL-2 mutein activities (FIG. 4 and Table 7). 589A-IL-2E and 589A-IL-2F molecules were assembled even more stably and displayed more than 4,000-fold of inhibition on the IL-2 mutein activities (FIG. 4 and Table 7). Potentially because of the higher affinity of the mask mutein D68E, 589A-IL-2E showed a better prodrug stability during the production than 589A-IL2-F (FIG. 4).

TABLE 7
CTLL2 Activities of 589A-IL-2 Prodrugs

| | CTLL2 Activity EC₅₀ (nM) | |
|---|---|---|
| Construct | Not activated | Activated |
| 589A-IL-2B | No activity | No activity |
| 589A-IL-2C | 8319 | 218 |
| 589A-IL-2E | No activity | 55 |
| 589A-IL-2F | 221036 | 53 |

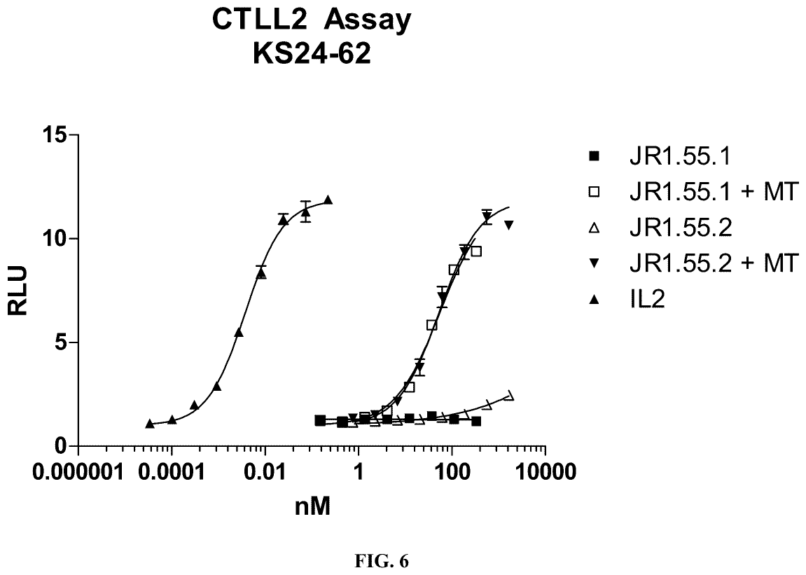

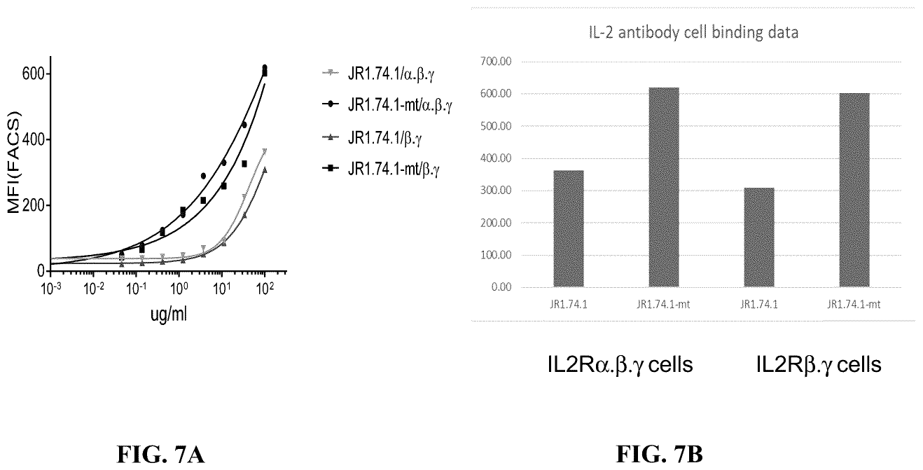

In a separate experiment, a new batch of 589A-IL-2E showed about 10-to 20-fold increase in binding to both HEK293-IL-2Rαβγ and HEK293-IL-2Rβγ cell lines after protease treatment (FIG. 6). In addition, 589A-IL-2E had similar bindings to HEK293-IL-2Rαβγ and HEK293-IL-2Rβγ, indicating that the a subunit did not contribute much to the binding of the IL-2 mutein and that the IL-2 mutein with mutations T3A/C125S/R38S/F42A/Y45A/E62A had significantly reduced binding affinity for IL-2Rα (FIGS. 7A and B).

The designs of the anti-PD-L1-IL-2 prodrugs are listed in Table 8.

TABLE 8
Designs of Anti-PD-L1-IL-2 Prodrugs

| | | Hole | | Cleavable | |
|---|---|---|---|---|---|
| | Knob | HC- | | Peptide | Mask |
| | HC-IL- | mask | LC | Linker | (IL-2Rβ |
| Construct | 2 SEQ | SEQ | SEQ | (SEQ) | ECD) |
| Anti- PD-L1-IL-2A | 191 | 192 | 189 | 217 | Dimer of |
| | | | | | WT |
| Anti-PD-L1-IL-2B | 191 | 193 | 189 | 18 | WT |
| Anti-PD-L1-IL-2C | 191 | 206 | 189 | 34 | WT |
| Anti-PD-L1-IL-2D | 191 | 207 | 189 | 35 | WT |
| Anti-PD-L1-IL-2E | 191 | 208 | 189 | 35 | D68E |

The anti-PD-L1-IL-2A molecule has two cleavage sites at its cleavable peptide linker and showed cleavage of the bands during the expression in HEK293, potentially due to the presence of proteases in cell culture media or in the cells (data not shown). Anti-PDL1-IL-2B showed correct assembly of the heterotetramer molecules and its purified sample showed significant activation after protease cleavage (FIG. 8). Anti-PD-L1-IL-2C, anti-PD-L1-IL-2D and anti-PD-L1-IL-2E were not assembled correctly and formed homodimers of HC-IL-2 (data not shown), and showed no inhibition of the IL-2 activities in the CTLL2 Assay. These data suggest that shorter cleavage linkers may interfere with the formation of the correct heterotetramer molecules.

### Example 5: ADCC Activity of 589A-IL-2 Mutein Fusion Molecules

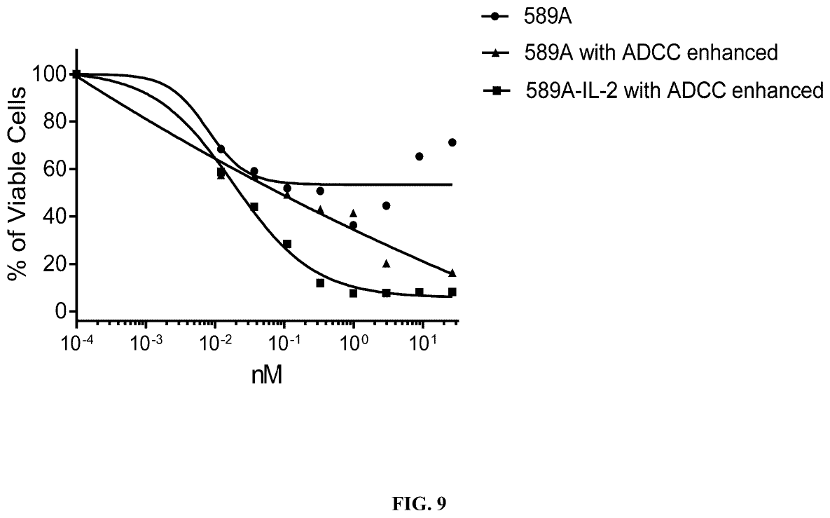

Anti-Claudin 18.2 antibody 589A, an afucoylsated form of 589A (af-589A), and the fusion of an IL-2 mutein to af-589A were tested for their in vitro activities in the ADCC assay, as described above. Af-589A had no or little fucose in its N-glycans and had enhanced ADCC function. The IL-2 mutein contained the mutations T3A/C125S/R38S/F42A/Y45A/E62A (SEQ ID NO: 10). The data show that addition of the IL-2 mutein to the 589A antibody further enhanced its ADCC activity (FIG. 9).

### Example 6: In Vivo Eficacy of 589A-IL-2 Prodrug

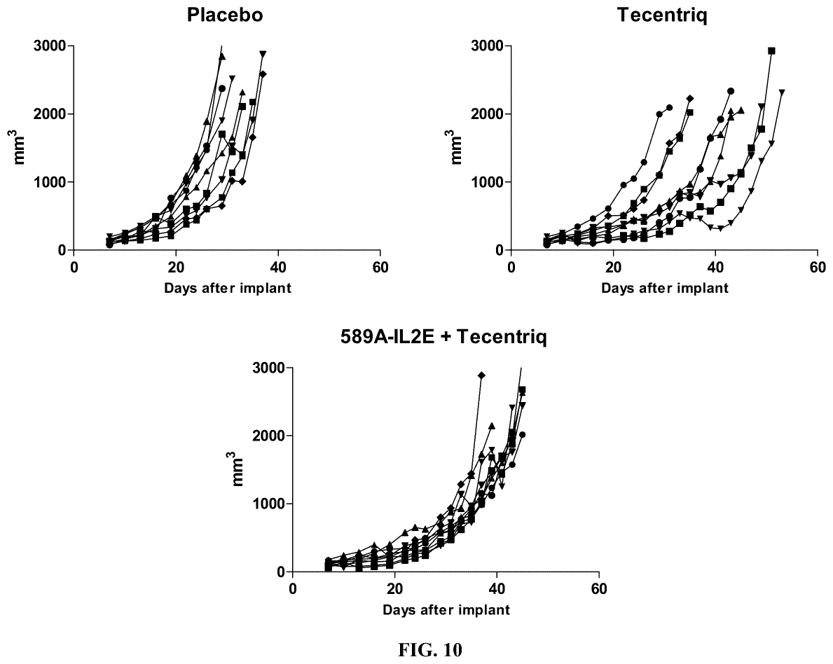

In vivo anti-cancer efficacy study was carried out with 589A-IL-2E in combination with an anti-PD-L1 antibody. Both said prodrug and the PD-L1 antibody were dosed subcutaneously at 10 mg/kg every other day. The CT26 mouse tumor cells transfected with human Claudin 18.2 were inoculated into the Balb/c mice. When the tumor size reached approximately 100 mm³, the mice were randomized into three groups based on their tumor sizes. Each mouse received buffer placebo (Group 1) (FIG. 10, top left panel), 10 mg/kg of the anti-PD-L1 antibody (Group2) (FIG. 10, top right panel), or 10 mg/kg of the anti-PD-L1 antibody plus 10 mg/kg of the 589A-IL-2E prodrug (Group 3) (FIG. 10, bottom panel) subcutaneously. Dosing was performed on day 7, 9, 11, 13, 15 and 18. The tumor sizes and body weights were monitored during the course of the study.

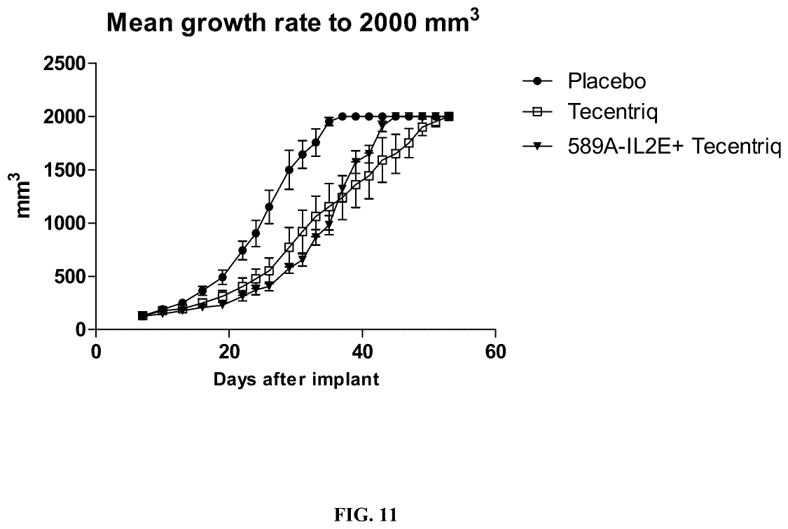

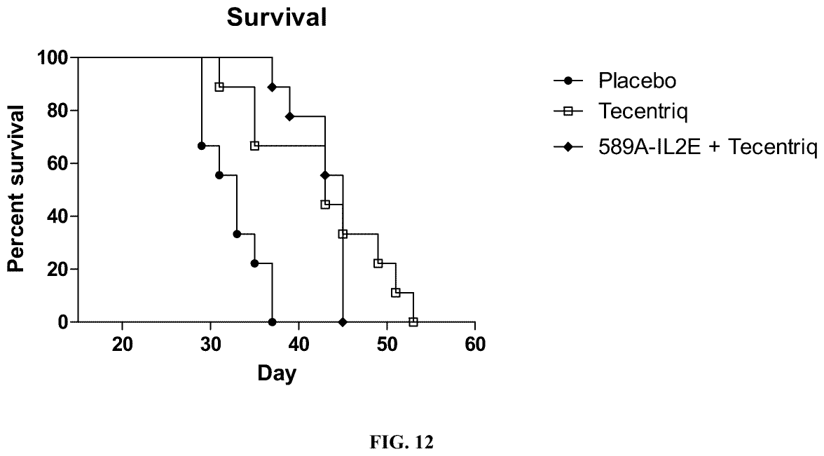

As shown in FIG. 10, the treatment group with both the prodrug and the antibodies had more homogeneous tumor sizes, compared to placebo and the PD-L1 antibody groups. As shown in FIG. 11, treatment groups with both the prodrug and the PD-L1 antibody had slowest tumor growth until approximately day 35, while the survival curves did not cross until day 42 (FIG. 12). The treatment was stopped at day 18. Without wishing to be bound by theory, we believe that one of the potential causes for the crossover with the PD-L1 antibody group was that the mice were wildtype and there could potentially be antibodies generated against 589A-IL-2E. 589A was a humanized antibody derived from rabbit B cell cloning.

The above non-limiting examples are provided for illustrative purposes only in order to facilitate a more complete understanding of the disclosed subject matter. These examples should not be construed to limit any of the embodiments described in the present specification, including those pertaining to the antibodies, pharmaceutical compositions, or methods and uses for treating cancer, a neurodegenerative or an infectious disease.

SEQUENCES
In the sequences below, boxed residues indicate mutations. Underlines in cleavable linkers
indicate protease substrate sequences.
SEQ ID NO: 1- human IL-2
APTSSSTKKTQLQLEHLLLDLQMILNGINNYKNPKLTRMLTFKFYMPKKATELKHLQCLEEELKPLEE
VLNLAQSKNFHLRPRDLISNINVIVLELKGSETTEMCEYADETATIVEFLNRWITFCQSIISTLT
SEQ ID NO: 2- human IL-15
GIHVFILGCFSAGLPKTEANWVNVISDLKKIEDLIQSMHIDATLYTESDVHPSCKVTAMKCFLLELQV
ISLESGDASIHDTVENLIILANNSLSSNGNVTESGCKECEELEEKNIKEFLQSFVHIVQMFINTS
SEQ ID NO: 3- Human IL-2 Receptor Beta Subunit Extracellular Domain
(https://www.uniprot.org/uniprot/P14784)
AVNGTSQFTCFYNSRANISCVWSQDGALQDTSCQVHAWPDRRRWNQTCELLPVSQASWACNLILGAPD
SQKLTTVDIVTLRVLCREGVRWRVMAIQDFKPFENLRLMAPISLQVVHVETHRCNISWEISQASHYFE
RHLEFEARTLSPGHTWEEAPLLTLKQKQEWICLETLTPDTQYEFQVRVKPLQGEFTTWSPWSQPLAFR
TKPAALGKDT
SEQ ID NO: 4- Human IL-2 Receptor Beta Subunit Extracellular Domain
Mutant D68E (https://www.uniprot.org/uniprot/P14784)

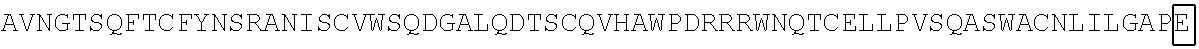

SQKLTTVDIVTLRVLCREGVRWRVMAIQDEKPFENLRLMAPISLQVVHVETHRCNISWEISQASHYFE
RHLEFEARTLSPGHTWEEAPLLTLKQKQEWICLETLTPDTQYEFQVRVKPLQGEFTTWSPWSQPLAFR
TKPAALGKDT
SEQ ID NO: 5- Human IL-2 Receptor Beta Subunit Extracellular Domain
Mutant E136Q/H138R (https://www.uniprot.org/uniprot/P14784)
AVNGTSQFTCFYNSRANISCVWSQDGALQDTSCQVHAWPDRRRWNQTCELLPVSQASWACNLILGAPD

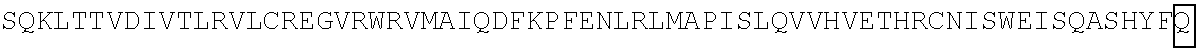

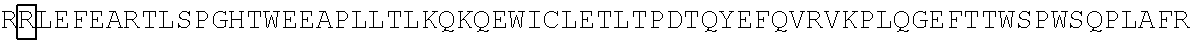

TKPAALGKDT
SEQ ID NO: 6- Human IL-2 Receptor Gamma Subunit Extracellular
Domain (http://www.uniprot.org/uniprot/P31785)
LNTTILTPNG NEDTTADFFL TTMPTDSLSV STLPLPEVQC FVENVEYMNC TWNSSSEPQP
TNLTLHYWYK NSDNDKVQKC SHYLFSEEIT SGCQLQKKEI HLYQTFVVQL QDPREPRRQA
TQMLKLQNLV IPWAPENLTL HKLSESQLEL NWNNRELNHC LEHLVQYRTD WDHSWTEQSV
DYRHKFSLPS VDGQKRYTFR VRSRENPLCG SAQHWSEWSH PIHWGSNTSK ENPFLFALEA
SEQ ID NO: 7- IL15 receptor alpha subunit sushi domain
ITCPPPMSVE HADIWVKSYS LYSRERYICN SGFKRKAGTS SLTECVLNKA TNVAHWTTPS
LKCIRDPALV HQRPAPP
SEQ ID NO: 8- IL-2 agonist polypeptide
APXₐₐ₃SSSTKKT QLQLEHLLLD LQMILNGINN YKNPXₐₐ₃₅LTRMLTXₐₐ₄₂KFXₐₐ₄₅MPKKA
TELKHLQCLE EELKPLEEVL NXₐₐ₇₂Xₐₐ₇₃QSKNFHL RPRDLISNIN VIVLELKGSE
TTFMCEYADE TATIVEFLNR WITFXₐₐ₁₂₅QSIIS TLT
wherein Xaa3 is N or A; wherein Xaa125 is C or S; wherein Xaa35 is
selected from K and N; wherein Xaa42 is selected from A, G, S, T, Q,
E, N, D, R, and K; wherein Xaa45 is selected from A, G, S, T, Q, E,
N, D, R, and K; wherein Xaa72 is selected from A, G, S, T, Q, E, N,
D, R, and K; and wherein Xaa73 is selected from A and T.
SEQ ID NO: 9- IL-2 agonist polypeptide with L72G
APASSSTKKTQLQLEHLLLDLQMILNGINNYKNPKLTRMLTAKFAMPKKATELKHLQCLEEELKPLEE

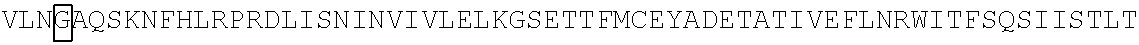

SEQ ID NO: 10- IL-2 agonist polypeptide mutein with T3A/C125S-
R38S/F42A/Y45A/E62A

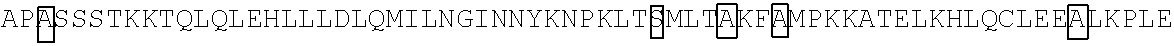

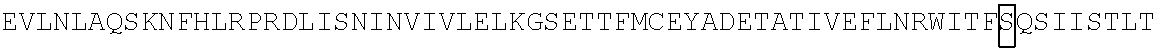

SEQ ID NO: 11- IL-2 agonist polypeptide mutein with C125S-
R38S/F42A/Y45A/E62A

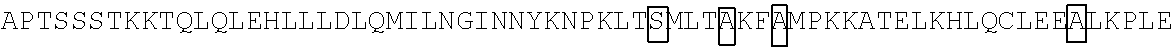

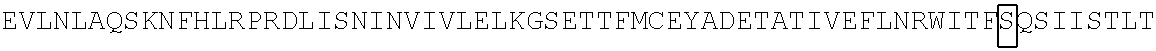

SEQ ID NO: 12- IL-2 agonist polypeptide mutein with T3A/C125S-
R38S/F42A/Y45A/E62L

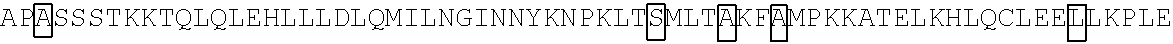

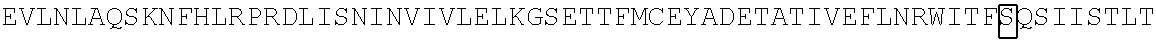

SEQ ID NO: 13- IL-2 agonist polypeptide mutein with T3A/C125S-
R38S/F42A/Y45A/E62L/E68V

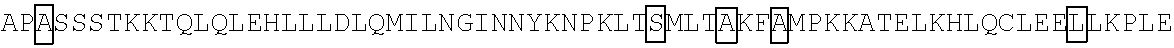

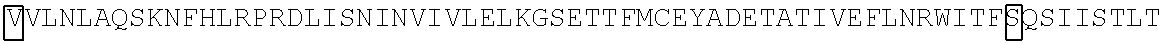

SEQ ID NO: 14- IL-2 agonist polypeptide mutein with T3A/C125S-
R38S/F42I/Y45A/E62A

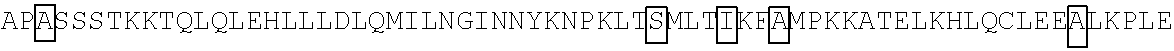

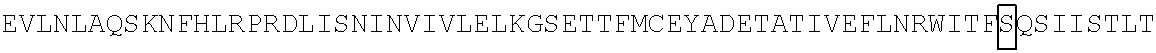

SEQ ID NO: 15- IL-2 agonist polypeptide mutein with T3A/C125S-
R38S/F42K/Y45A/E62A

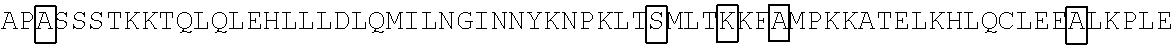

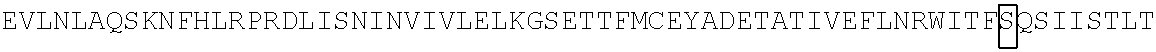

SEQ ID NO: 16- IL-2 agonist polypeptide mutein with T3A/C125S-
R38S/F42K/Y45N/E62A

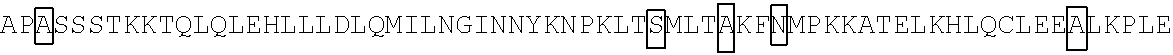

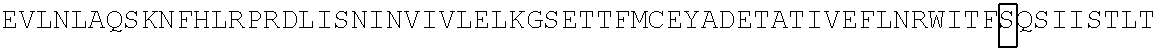

SEQ ID NO: 17- IL-2 agonist polypeptide mutein with T3A/C125S-
R38S/F42A/Y45R/E62A

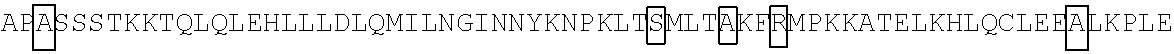

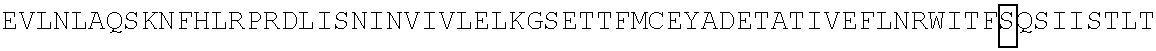

SEQ ID NO: 18- cleavable linker
GGGGSGGGGSGGGGSLSGRSDNHGGGGS
SEQ ID NO: 19- IL-2 agonist polypeptide mutein with T3A/C125S-
R38S/F42K/Y45A/E62A/E68V

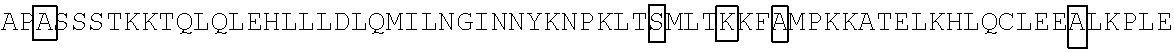

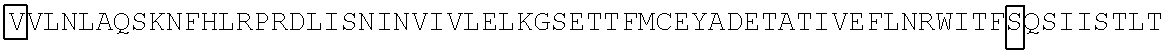

SEQ ID NO: 20- IL-2 agonist polypeptide mutein with T3A/C125S-
R38S/F42A/Y45N/E62A/E68V

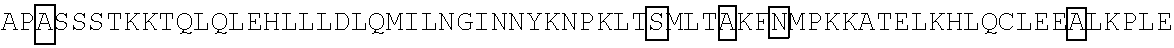

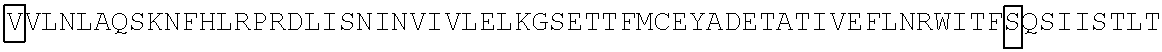

SEQ ID NO: 21- IL-2 agonist polypeptide mutein with T3A/C125S-
R38S/F42A/Y45R/E62A/E68V

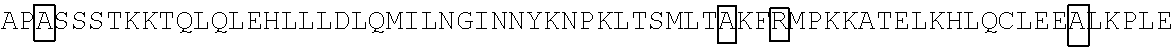

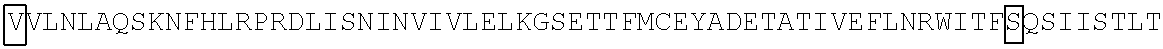

SEQ ID NO: 22- IL-2 agonist polypeptide mutein with
T3A/R38S/F42A/Y45A/A73T/C125S

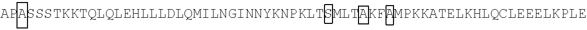

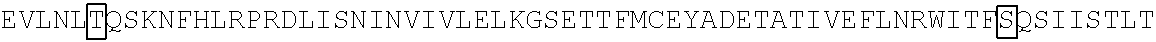

SEQ ID NO: 23- IL-2 agonist polypeptide mutein with
T3A/K35N/R38S/F42A/Y45A/A73T/C125S

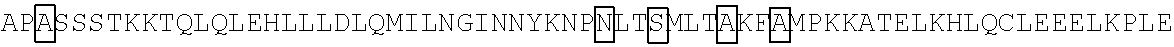

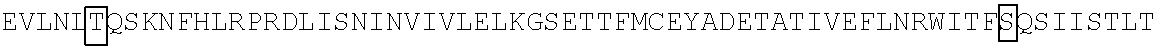

SEQ ID NO: 24- IL-2 agonist polypeptide mutein with
T3A/R38S/F42I/Y45A/A73T/C125S

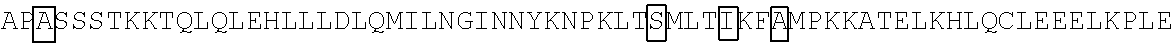

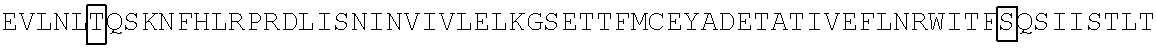

SEQ ID NO: 25- IL-2 agonist polypeptide mutein with
T3A/K35N/R38S/F42I/Y45A/A73T/C125S

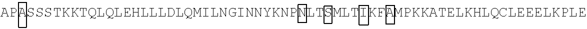

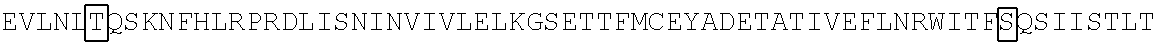

SEQ ID NO: 26- IL-2 agonist polypeptide mutein with
T3A/R38S/F42K/Y45A/A73T/C125S

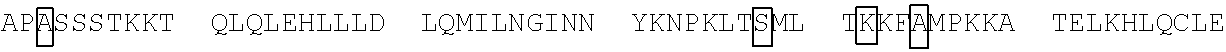

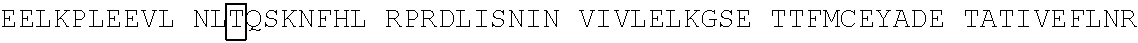

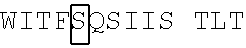

SEQ ID NO: 27- IL-2 agonist polypeptide mutein with
T3A/K35N/R38S/F42K/Y45A/A73T/C125S

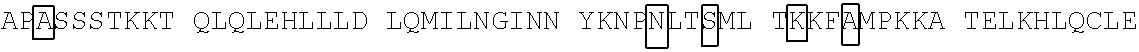

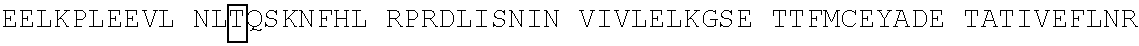

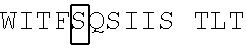

SEQ ID NO: 28- IL-2 agonist polypeptide mutein with
T3A/R38S/F42A/Y45N/A73T/C125S

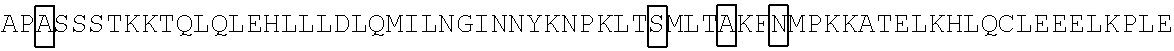

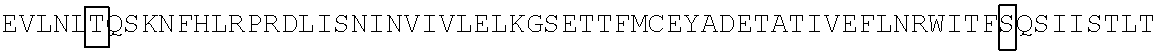

SEQ ID NO: 29- IL-2 agonist polypeptide mutein with
T3A/R38S/F42A/Y45R/A73T/C125S

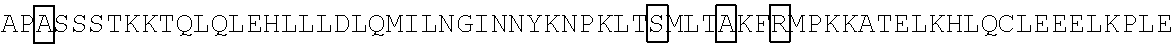

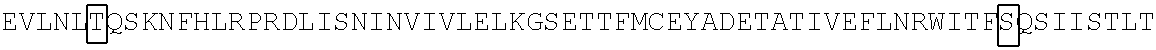

SEQ ID NO: 30- IL-2 agonist polypeptide mutein with
T3A/R38S/F42A/Y45A/E62A/C125S/Q126W

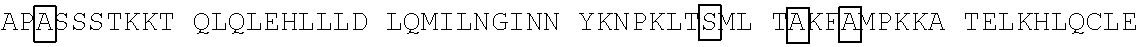

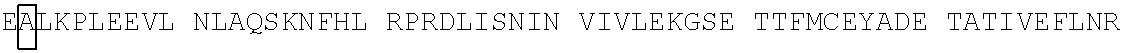

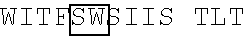

SEQ ID NO: 31- IL-2 agonist polypeptide mutein with
T3A/R38S/F42K/Y45A/E62A/A73T/C125S

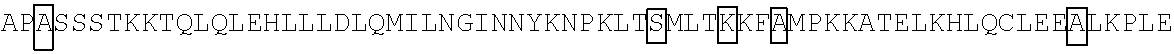

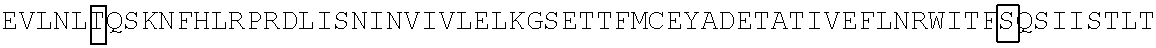

SEQ ID NO: 32- IL-2 agonist polypeptide mutein with
T3A/R38S/F42A/Y45N/E62A/A73T/C125S

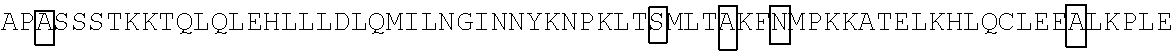

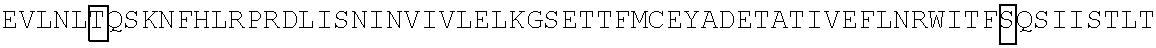

SEQ ID NO: 33- IL-2 agonist polypeptide mutein with
T3A/R38S/F42A/Y45R/E62A/A73T/C125S

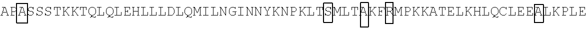

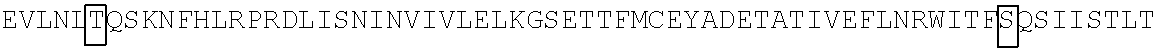

SEQ ID NO: 34- cleavable peptide linker
GGSLSGRSDNHGGGGS
SEQ ID NO: 35- cleavable peptide linker
GGSLSGRSDNHGS
SEQ ID NO: 36- IL-2 agonist polypeptide mutein with
T3A/K35N/R38S/F42A/Y45N/A73T/C125S

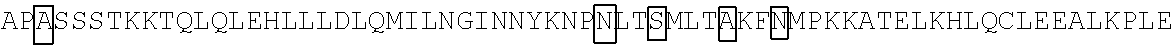

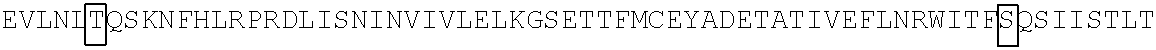

SEQ ID NO: 37- IL-2 agonist polypeptide mutein

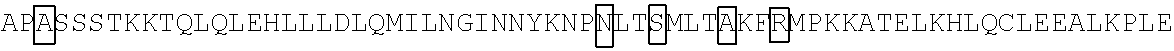

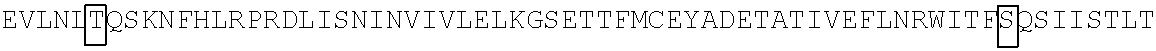

SEQ ID NO: 38- cleavable linker
GGGGSGGGGSGGGGSISSGLLSSGGSGGSLSGRSDNHGGGGS
SEQ ID NO: 39- IL-2 agonist polypeptide mutein

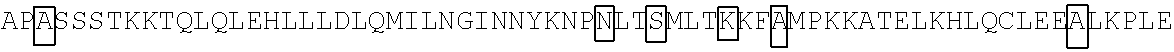

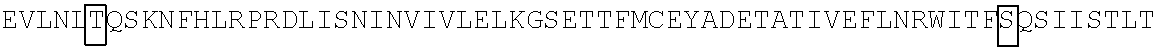

SEQ ID NO: 40- IL-2 agonist polypeptide mutein

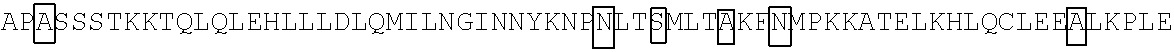

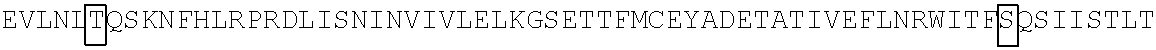

SEQ ID NO: 41- IL-2 agonist polypeptide mutein

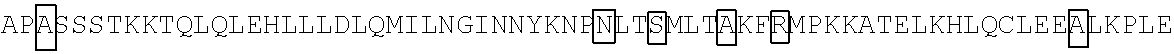

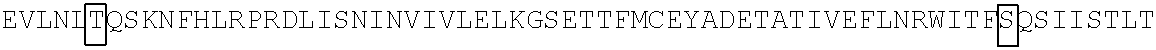

SEQ ID NO: 42- IL-2 agonist polypeptide mutein with Q126W

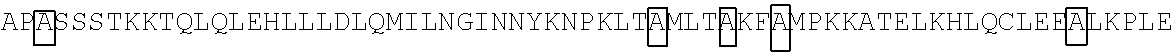

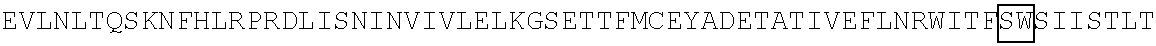

SEQ ID NO: 43- IL-2 agonist polypeptide mutein with
T3A/C125S/R38S/F42A/Y45A/E62A/N88A/Q126H

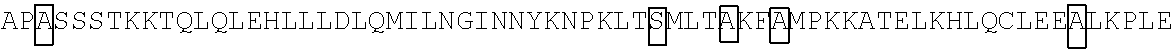

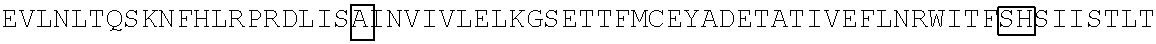

SEQ ID NO: 44- IL-2 agonist polypeptide mutein with
T3A/C125S/R38S/F42A/Y45A/E62A/Q126A

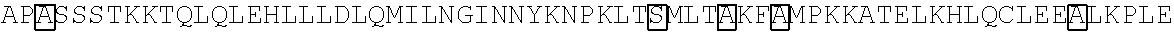

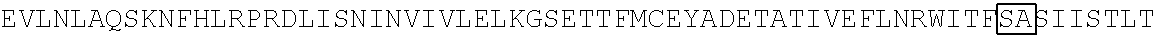

SEQ ID NO: 45- IL-2 agonist polypeptide mutein with Q126W

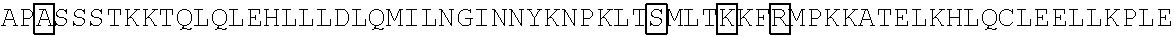

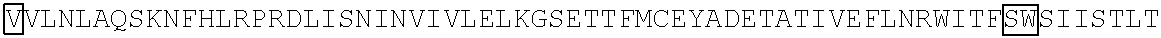

SEQ ID NO: 46- IL-2 agonist polypeptide mutein with Q126W

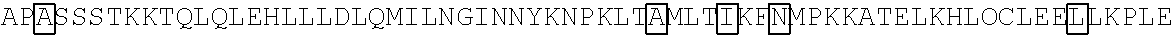

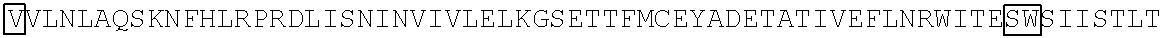

SEQ ID NO: 47-51 Peptide Linker
(SEQ ID NO: 47)
GGGGS
(SEQ ID NO: 48)
GGGGSGGGGS
(SEQ ID NO: 49)
GGGGSGGGGSGGGGS
GGGGSGGGGXGGGGSGGGGS (SEQ ID NO: 50), X = A or N
GGGGSGGGGXGGGGYGGGGS (SEQ ID NO: 51), X = S, A or N, and Y = A or N
SEQ ID NO: 52-121- Cleavable peptide linkers
(SEQ ID NO: 52)
LSGRSDNH
(SEQ ID NO: 53)
ISSGLLSS
(SEQ ID NO: 54)
GPLGVR
(SEQ ID NO: 55)
SGRSA
(SEQ ID NO: 56)
GGGGSISSGLLSSGGSGGSLGGSGRSANAILEGGGGSGGGGS
(SEQ ID NO: 57)
GGGGSISSGLLSSGGSLGGSGRSANAILEGGGGS
(SEQ ID NO: 58)
GGGGSLGGSGRSANAILEGGSGGSISSGLLSSGGGGS
(SEQ ID NO: 59)
GGGGSLGGSGRSANAILEGGSISSGLLSSGGGGS
(SEQ ID NO: 60)
GGGGSLGGSGRSANAILEGGSGGSISSGLLSSGGGGSGGGGS
(SEQ ID NO: 61)
GGGGSLGGSGRSANAILEGGGGSGGGGSGGGGS
(SEQ ID NO: 62)
GGGGSGGGGSGGGGSISSGLLSSGGGGS
(SEQ ID NO: 63)
GGGGSLSGRSDNHGGGGS
(SEQ ID NO: 64)
(GGGGS) ₙGGWHTGRN (GGGGS)ₘ
(SEQ ID NO: 65)
(GGGGS) ₙTGRGPSWV (GGGGS)ₘ
(SEQ ID NO: 66)
(GGGGS) ₙSARGPSRW (GGGGS)ₘ
(SEQ ID NO: 67)
(GGGGS) ₙTARGPSFK (GGGGS)ₘ
(SEQ ID NO: 68)
(GGGGS) ₙTARGPSW (GGGGS)ₘ
(SEQ ID NO: 69)
(GGGGS) ₙLSGRSDNH (GGGGS)ₘ
(SEQ ID NO: 70)
(GGGGS) ₙLGGSGRSANAILEGPLGVR (GGGGS)m
(SEQ ID NO: 71)
(GGGGS) ₙLGGSGRSANAILEGGSGPLGVR (GGGGS)m
(SEQ ID NO: 72)
(GGGGS) ₙLGGSGRSANAILEGGSGGSGPLGVR (GGGGS)m
(SEQ ID NO: 73)
(GGGGS) ₙISSGLLSSLSGRSDNH (GGGGS)ₘ
(SEQ ID NO: 74)
(GGGGS) ₙISSGLLSSGGSLSGRSDNH (GGGGS)ₘ
(SEQ ID NO: 75)
(GGGGS) ₙISSGLLSSGGSGGGSLSGRSDNH (GGGGS)ₘ
wherein n = 0, 1, 2, 3, or 4; and wherein m = 0, 1, 2, 3, or 4
(SEQ ID NO: 76)
VNGGGGSGPLGVRAAQPA
(SEQ ID NO: 77)
GGGGSGPLGVRGGGGS
(SEQ ID NO: 78)
GGGGSGPLGVRGGS
(SEQ ID NO: 79)
(GGGGS) n1 (QGQSGQ) n2 PLGL (GGGGS) n3
(SEQ ID NO: 80)
(GGGGS) n1 (QGQSGQ) n2 HTGRSGAL (GGGGS) n3
(SEQ ID NO: 81)
(GGGGS) n1 (QGQSGQ) n2 PLTGRSGG (GGGGS) n3
(SEQ ID NO: 82)
(GGGGS) n1 (QGQSGQ) n2 AARGPATH (GGGGS) n3
(SEQ ID NO: 83)
(GGGGS) n1 (QGQSGQ) n2 RGPAFNPM (GGGGS) n3
(SEQ ID NO: 84)
(GGGGS) n1 (QGQSGQ) n2 SSRGPAYL (GGGGS) n3
(SEQ ID NO: 85)
(GGGGS) n1 (QGQSGQ) n2 RGPATPIM (GGGGS) n3
(SEQ ID NO: 86)
(GGGGS) n1 (QGQSGQ) n2 RGPA (GGGGS) n3
(SEQ ID NO: 87)
(GGGGS) n1 (QGQSGQ) n2 GGQPSGMWGW (GGGGS) n3
(SEQ ID NO: 88)
(GGGGS) n1 (QGQSGQ) n2 FPRPLGITGL (GGGGS) n3
(SEQ ID NO: 89)
(GGGGS) n1 (QGQSGQ) n2 VILMPLGFLGP (GGGGS) n3
(SEQ ID NO: 90)
(GGGGS) n1 (QGQSGQ) n2 SPLTGRSG (GGGGS) n3
(SEQ ID NO: 91)
(GGGGS) n1 (QGQSGQ) n2 SAGESLPA (GGGGS) n3
(SEQ ID NO: 92)
(GGGGS) n1 (QGQSGQ) n2 LAPLGLQRR (GGGGS) n3
(SEQ ID NO: 93)
(GGGGS) n1 (QGQSGQ) n2 SGGPLGVR (GGGGS) n3
(SEQ ID NO: 94)
(GGGGS) n1 (QGQSGQ) n2 GPLGVR (GGGGS) n3
(SEQ ID NO: 95)
(GGGGS) n1 (QGQSGQ) n2 ISSGLLSS (GGGGS) n3
(SEQ ID NO: 96)
(GGGGS) n1 (QGQSGQ) n2 QNQALRMA (GGGGS) n3
(SEQ ID NO: 97)
(GGGGS) n1 (QGQSGQ) n2 AQNLLGMV (GGGGS) n3
(SEQ ID NO: 98)
(GGGGS) n1 (QGQSGQ) n2 STFPFGMF (GGGGS) n3
(SEQ ID NO: 99)
(GGGGS) n1 (QGQSGQ) n2 PVGYTSSL (GGGGS) n3
(SEQ ID NO: 100)
(GGGGS) n1 (QGQSGQ) n2 DWLYWPGI (GGGGS) n3
(SEQ ID NO: 101)
(GGGGS) n1 (QGQSGQ) n2 MIAPVAYR (GGGGS) n3
(SEQ ID NO: 102)
(GGGGS) n1 (QGQSGQ) n2 RPSPMWAY (GGGGS) n3
(SEQ ID NO: 103)
(GGGGS) n1 (QGQSGQ) n2 WATPRPMR (GGGGS) n3
(SEQ ID NO: 104)
(GGGGS) n1 (QGQSGQ) n2 FRLLDWQW (GGGGS) n3
(SEQ ID NO: 105)
(GGGGS) n1 (QGQSGQ) n2 LKAAPRWA (GGGGS) n3
(SEQ ID NO: 106)
(GGGGS) n1 (QGQSGQ) n2 GPSHLVLT (GGGGS) n3
(SEQ ID NO: 107)
(GGGGS) n1 (QGQSGQ) n2 LPGGLSPW (GGGGS n3
(SEQ ID NO: 108)
(GGGGS) n1 (QGQSGQ) n2 MGLFSEAG (GGGGS) n3
(SEQ ID NO: 109)
(GGGGS) n1 (QGQSGQ) n2 SPLPLRVP (GGGGS) n3
(SEQ ID NO: 110)
(GGGGS) n1 (QGQSGQ) n2 RMHLRSLG (GGGGS) n3
(SEQ ID NO: 111)
(GGGGS) n1 (QGQSGQ) n2 LAAPLGLL (GGGGS) n3
(SEQ ID NO: 112)
(GGGGS) n1 (QGQSGQ) n2 AVGLLAPP (GGGGS) n3
(SEQ ID NO: 113)
(GGGGS) n1 (QGQSGQ) n2 LLAPSHRA (GGGGS) n3
(SEQ ID NO: 114)
(GGGGS) n1 (QGQSGQ) n2 PAGLWLDP (GGGGS) n3
(SEQ ID NO: 115)
(GGGGS) n1 (QGQSGQ) n2 ISSGLSS (GGGGS) n3
(SEQ ID NO: 116)
(GGGGS) n1 ISSGLLSSGGSGGSLSGRSDNH (GGGGS) n3
(SEQ ID NO: 117)
(GGGGS) n1 LSGRSDNHGGSGGSISSGLLSS (GGGGS) n3
(SEQ ID NO: 118)
(QGQSGQ) n2 LSGRSDNH (GGGGS) n3
(SEQ ID NO: 119)
(GGGGS) n1 (QGQSGQ) n2 TARGPSFK (GGGGS) n3
(SEQ ID NO: 120)
(GGGGS) n1 (QGQSGQ) n2 TARGPSW (GGGGS) n3
(SEQ ID NO: 121)
(GGGGS) n1 (QGQSGQ) n2 GGWHTGRN (GGGGS) n3
wherein n1 = 0, 1, 2, 3, or 4; n2 = 0 or 1; and n3 = 0, 1, 2, 3, or
4
SEQ ID NO: 122- Human IL-7 amino acid sequence
DCDIEGKDGKQYESVLMVSIDQLLDSMKEIGSNCLNNEFNFFKRHICDANKEGMELFRA
ARKLRQFLKMNSTGDFDLHLLKVSEGTTILLNCTGQVKGRKPAALGEAQPTKSLEENKS
LKEQKKLNDLCFLKRLLQEIKTCWNKILMGTKEH
SEQ ID NO: 123- Human CCL19 amino acid sequence
TNDAEDCC LSVTQKPIPG YIVRNFHYLL IKDGCRVPAV VETTLRGRQL CAPPDQPWVE
RIIQRLQRTS AKMKRRSS
SEQ ID NO: 124- human albumin
DAHKSEVAHRFKDLGEENFKALVLIAFAQYLQQCPFEDHVKLVNEVTEFAKTCVADESAENCDKSLHT
LFGDKLCTVATLRETYGEMADCCAKQEPERNECFLQHKDDNPNLPRLVRPEVDVMCTAFHDNEETELK
KYLYEIARRHPYFYAPELLFFAKRYKAAFTECCQAADKAACLLPKLDELRDEGKASSAKQRLKCASLQ
KFGERAFKAWAVARLSQRFPKAEFAEVSKLVTDLTKVHTECCHGDLLECADDRADLAKYICENQDSIS
SKLKECCEKPLLEKSHCIAEVENDEMRADLPSLAADFVESKDVCKNYAEAKDVFLGMFLYEYARRHPD
YSVVLLLRLAKTYETTLEKCCAAADPHECYAKVEDEFKPLVEEPQNLIKQNCELFEQLGEYKFQNALL
VRYTKKVPQVSTPTLVEVSRNLGKVGSKCCKHPEAKRMPCAEDYLSVVLNQLCVLHEKTPVSDRVTKC
CTESLVNRRPCFSALEVDETYVPKEFNAETFTFHADICTLSEKERQIKKQTALVELVKHKPKATKEQL
KAVMDDFAAFVEKCCKADDKETCFAEEGKKLVAASQAALGL
SEQ ID NO: 125- human albumin with enhanced FCRN binding affinity
(K573P)
DAHKSEVAHRFKDLGEENFKALVLIAFAQYLQQCPFEDHVKLVNEVTEFAKTCVADESAENCDKSLHT
LFGDKLCTVATLRETYGEMADCCAKQEPERNECFLQHKDDNPNLPRLVRPEVDVMCTAFHDNEETFLK
KYLYEIARRHPYFYAPELLFFAKRYKAAFTECCQAADKAACLLPKLDELRDEGKASSAKQRLKCASLQ
KFGERAFKAWAVARLSQRFPKAEFAEVSKLVTDLTKVHTECCHGDLLECADDRADLAKYICENQDSIS
SKLKECCEKPLLEKSHCIAEVENDEMRADLPSLAADFVESKDVCKNYAEAKDVELGMELYEYARRHPD
YSVVLLLRLAKTYETTLEKCCAAADPHECYAKVFDEFKPLVEEPQNLIKQNCELFEQLGEYKFQNALL
VRYTKKVPQVSTPTLVEVSRNLGKVGSKCCKHPEAKRMPCAEDYLSVVLNQLCVLHEKTPVSDRVTKC
CTESLVNRRPCFSALEVDETYVPKEFNAETFTFHADICTLSEKERQIKKQTALVELVKHKPKATKEQL
KAVMDDFAAFVEKCCKADDKETCFAEEGPKLVAASQAALGL
SEQ ID NO: 126- HSA- IL-2-T3A/C125S-F42A/Y45A/L72G
DAHKSEVAHRFKDLGEENFKALVLIAFAQYLQQCPFEDHVKLVNEVTEFAKTCVADESAENCDKSLHT
LFGDKLCTVATLRETYGEMADCCAKQEPERNECFLQHKDDNPNLPRLVRPEVDVMCTAFHDNEETFLK
KYLYEIARRHPYFYAPELLFFAKRYKAAFTECCQAADKAACLLPKLDELRDEGKASSAKQRLKCASLQ
KFGERAFKAWAVARLSQRFPKAEFAEVSKLVTDLTKVHTECCHGDLLECADDRADLAKYICENQDSIS
SKLKECCEKPLLEKSHCIAEVENDEMPADLPSLAADFVESKDVCKNYAEAKDVELGMELYEYARRHPD
YSVVLLLRLAKTYETTLEKCCAAADPHECYAKVFDEFKPLVEEPQNLIKQNCELFEQLGEYKFQNALL
VRYTKKVPQVSTPTLVEVSRNLGKVGSKCCKHPEAKRMPCAEDYLSVVLNQLCVLHEKTPVSDRVTKC
CTESLVNRRPCFSALEVDETYVPKEFNAETFTFHADICTLSEKERQIKKQTALVELVKHKPKATKEQL
KAVMDDFAAFVEKCCKADDKETCFAEEGKKLVAASQAALGLGGGGSGGGGSGGGGSAPASSSTKKTQL
QLEHLLLDLQMILNGINNYKNPKLTRMLTAKFAMPKKATELKHLQCLEEELKPLEEVLNGAQSKNFHL
RPRDLISNINVIVLELKGSETTFMCEYADETATIVEFLNRWITESQSIISTLT
SEQ ID NO: 127- HSA- IL-2-T3A/C125S-R38S/F42A/Y45A/E62A
DAHKSEVAHRFKDLGEENFKALVLIAFAQYLQQCPFEDHVKLVNEVTEFAKTCVADESAENCDKSLHT
LFGDKLCTVATLRETYGEMADCCAKQEPERNECFLQHKDDNPNLPRLVRPEVDVMCTAFHDNEETFLK
KYLYEIARRHPYFYAPELLFFAKRYKAAFTECCQAADKAACLLPKLDELRDEGKASSAKQRLKCASLQ
KFGERAFKAWAVARLSQRFPKAEFAEVSKLVTDLTKVHTECCHGDLLECADDRADLAKYICENQDSIS
SKLKECCEKPLLEKSHCIAEVENDEMPADLPSLAADFVESKDVCKNYAEAKDVELGMELYEYARRHPD
YSVVLLLRLAKTYETTLEKCCAAADPHECYAKVEDEFKPLVEEPQNLIKQNCELFEQLGEYKFQNALL
VRYTKKVPQVSTPTLVEVSRNLGKVGSKCCKHPEAKRMPCAEDYLSVVLNQLCVLHEKTPVSDRVTKC
CTESLVNRRPCFSALEVDETYVPKEFNAETFTFHADICTLSEKERQIKKQTALVELVKHKPKATKEQL
KAVMDDFAAFVEKCCKADDKETCFAEEGKKLVAASQAALGLGGGGSGGGGSGGGGSAPASSSTKKTQL
QLEHLLLDLQMILNGINNYKNPKLTSMLTAKFAMPKKATELKHLQCLEEALKPLEEVLNLAQSKNFHL
RPRDLISNINVIVLELKGSETTFMCEYADETATIVEFLNRWITESQSIISTLT
SEQ ID NO: 128- HSA- IL-2-T3A/C125S-R38S/F42A/Y45A/E62A/N88A/Q126H
DAHKSEVAHRFKDLGEENFKALVLIAFAQYLQQCPFEDHVKLVNEVTEFAKTCVADESAENCDKSLHT
LFGDKLCTVATLRETYGEMADCCAKQEPERNECFLQHKDDNPNLPRLVRPEVDVMCTAFHDNEETFLK
KYLYEIARRHPYFYAPELLFFAKRYKAAFTECCQAADKAACLLPKLDELRDEGKASSAKQRLKCASLQ
KFGERAFKAWAVARLSQRFPKAEFAEVSKLVTDLTKVHTECCHGDLLECADDRADLAKYICENQDSIS
SKLKECCEKPLLEKSHCIAEVENDEMPADLPSLAADFVESKDVCKNYAEAKDVELGMELYEYARRHPD
YSVVLLLRLAKTYETTLEKCCAAADPHECYAKVEDEFKPLVEEPQNLIKQNCELFEQLGEYKFQNALL
VRYTKKVPQVSTPTLVEVSRNLGKVGSKCCKHPEAKRMPCAEDYLSVVLNQLCVLHEKTPVSDRVTKC
CTESLVNRRPCFSALEVDETYVPKEFNAETFTFHADICTLSEKERQIKKQTALVELVKHKPKATKEQL
KAVMDDFAAFVEKCCKADDKETCFAEEGKKLVAASQAALGLGGGGSGGGGSGGGGSAPASSSTKKTQL
QLEHLLLDLQMILNGINNYKNPKLTSMLTAKFAMPKKATELKHLQCLEEALKPLEEVLNLAQSKNFHL
RPRDLISAINVIVLELKGSETTEMCEYADETATIVEFLNRWITFSHSIISTLT
SEQ ID NO: 129- HSA-IL-2-T3A/C125S-R38S/F42A/Y45A/E62A/Q126W (No
linker)
DAHKSEVAHRFKDLGEENFKALVLIAFAQYLQQCPFEDHVKLVNEVTEFAKTCVADESAENCDKSLHT
LFGDKLCTVATLRETYGEMADCCAKQEPERNECFLQHKDDNPNLPRLVRPEVDVMCTAFHDNEETFLK
KYLYEIARRHPYFYAPELLFFAKRYKAAFTECCQAADKAACLLPKLDELRDEGKASSAKQRLKCASLQ
KFGERAFKAWAVARLSQRFPKAEFAEVSKLVTDLTKVHTECCHGDLLECADDRADLAKYICENQDSIS
SKLKECCEKPLLEKSHCIAEVENDEMPADLPSLAADEVESKDVCKNYAEAKDVELGMELYEYARRHPD
YSVVLLLRLAKTYETTLEKCCAAADPHECYAKVEDEFKPLVEEPQNLIKQNCELFEQLGEYKFQNALL
VRYTKKVPQVSTPTLVEVSRNLGKVGSKCCKHPEAKRMPCAEDYLSVVLNQLCVLHEKTPVSDRVTKC
CTESLVNRRPCFSALEVDETYVPKEFNAETFTFHADICTLSEKERQIKKQTALVELVKHKPKATKEQL
KAVMDDFAAFVEKCCKADDKETCFAEEGKKLVAASQAALGLAPASSSTKKTQLQLEHLLLDLQMILNG
INNYKNPKLTSMLTAKFAMPKKATELKHLQCLEEALKPLEEVLNLAQSKNFHLRPRDLISNINVIVLE
LKGSETTFMCEYADETATIVEFLNRWITFSWSIISTLT
SEQ ID NO: 130- HSA-IL-2-T3A/C125S-R38S/F42A/Y45A/E62A/Q126W
DAHKSEVAHRFKDLGEENFKALVLIAFAQYLQQCPFEDHVKLVNEVTEFAKTCVADESAENCDKSLHT
LFGDKLCTVATLRETYGEMADCCAKQEPERNECFLQHKDDNPNLPRLVRPEVDVMCTAFHDNEETFLK
KYLYEIARRHPYFYAPELLFFAKRYKAAFTECCQAADKAACLLPKLDELRDEGKASSAKQRLKCASLQ
KFGERAFKAWAVARLSQRFPKAEFAEVSKLVTDLTKVHTECCHGDLLECADDRADLAKYICENQDSIS
SKLKECCEKPLLEKSHCIAEVENDEMPADLPSLAADEVESKDVCKNYAEAKDVELGMELYEYARRHPD
YSVVLLLRLAKTYETTLEKCCAAADPHECYAKVEDEFKPLVEEPQNLIKQNCELFEQLGEYKFQNALL
VRYTKKVPQVSTPTLVEVSRNLGKVGSKCCKHPEAKRMPCAEDYLSVVLNQLCVLHEKTPVSDRVTKC
CTESLVNRRPCFSALEVDETYVPKEFNAETFTFHADICTLSEKERQIKKQTALVELVKHKPKATKEQL
KAVMDDFAAFVEKCCKADDKETCFAEEGKKLVAASQAALGLGGGGSGGGGSGGGGSAPASSSTKKTQL
QLEHLLLDLQMILNGINNYKNPKLTSMLTAKFAMPKKATELKHLQCLEEALKPLEEVLNLAQSKNFHL
RPRDLISNINVIVLELKGSETTFMCEYADETATIVEFLNRWITFSWSIISTLT
SEQ ID NO: 131- HSA-IL-2-T3A/C125S-R38S/F42A/Y45A/E62A/Q126A, No
linker
DAHKSEVAHRFKDLGEENFKALVLIAFAQYLQQCPFEDHVKLVNEVTEFAKTCVADESAENCDKSLHT
LFGDKLCTVATLRETYGEMADCCAKQEPERNECFLQHKDDNPNLPRLVRPEVDVMCTAFHDNEETFLK
KYLYEIARRHPYFYAPELLFFAKRYKAAFTECCQAADKAACLLPKLDELRDEGKASSAKQRLKCASLQ
KFGERAFKAWAVARLSQRFPKAEFAEVSKLVTDLTKVHTECCHGDLLECADDRADLAKYICENQDSIS
SKLKECCEKPLLEKSHCIAEVENDEMPADLPSLAADFVESKDVCKNYAEAKDVFLGMFLYEYARRHPD
YSVVLLLRLAKTYETTLEKCCAAADPHECYAKVFDEFKPLVEEPQNLIKQNCELFEQLGEYKFQNALL
VRYTKKVPQVSTPTLVEVSRNLGKVGSKCCKHPEAKRMPCAEDYLSVVLNQLCVLHEKTPVSDRVTKC
CTESLVNRRPCFSALEVDETYVPKEFNAETFTFHADICTLSEKERQIKKQTALVELVKHKPKATKEQL

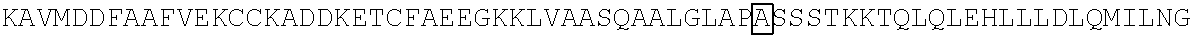

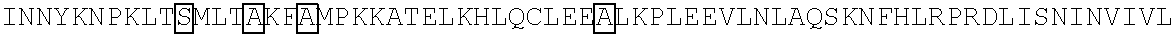

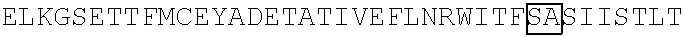

SEQ ID NO: 132- IgG1FC (with LALA and Knob)-IL-2-T3A/C125S-
R38S/F42A/Y45A/E62A
DKTHTCPPCPAPEAAGGPSVFLFPPKPKDTLMISRTPEVTCVVVDVSHEDPEVKENWYVDGVEVHNAK
TKPREEQYNSTYRVVSVLTVLHQDWLNGKEYKCKVSNKALPAPIEKTISKAKGQPREPQVYTLPPSRE
EMTKNQVSLYCLVKGFYPSDIAVEWESNGQPENNYKTTPPVLDSDGSFFLYSKLTVDKSRWQQGNVES
CSVMHEALHNHYTQKSLSLSPGKGGGGSGGGGSGGGGSAPASSSTKKTQLQLEHLLLDLQMILNGINN
YKNPKLTSMLTAKFAMPKKATELKHLQCLEEALKPLEEVLNLAQSKNFHLRPRDLISNINVIVLELKG
SETTFMCEYADETATIVEFLNRWITFSQSIISTLT
SEQ ID NO: 133- IgG1FC (with LALA and Knob)-IL-2-T3A/C125S-
R38A/F42R/Y45K/E62A
DKTHTCPPCPAPEAAGGPSVELFPPKPKDTLMISRTPEVTCVVVDVSHEDPEVKENWYVDGVEVHNAK
TKPREEQYNSTYRVVSVLTVLHQDWLNGKEYKCKVSNKALPAPIEKTISKAKGQPREPQVYTLPPSRE
EMTKNQVSLYCLVKGFYPSDIAVEWESNGQPENNYKTTPPVLDSDGSFFLYSKLTVDKSRWQQGNVES

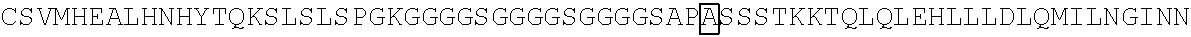

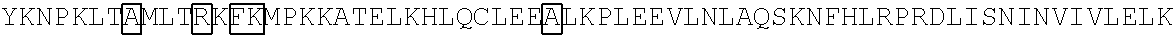

GSETTFMCEYADETATIVEFLNRWITFSQSIISTLT
SEQ ID NO: 134- IgG1FC (with LALA and Knob)-IL-2-T3A/C125S-
R38S/F42A/Y45A/E62A/Q126H
DKTHTCPPCPAPEAAGGPSVFLFPPKPKDTLMISRTPEVTCVVVDVSHEDPEVKENWYVDGVEVHNAK
TKPREEQYNSTYRVVSVLTVLHQDWLNGKEYKCKVSNKALPAPIEKTISKAKGQPREPQVYTLPPSRE
EMTKNQVSLTCLVKGFYPSDIAVEWESNGQPENNYKTTPPVLDSDGSFFLYSKLTVDKSRWQQGNVES

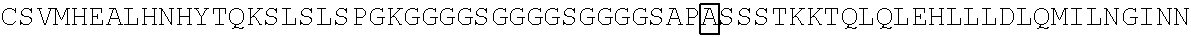

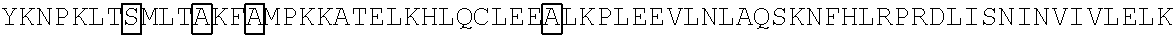

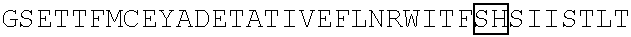

SEQ ID NO: 135- IgG1FC (with LALA and Knob)-IL-2-T3A/C125S-
R38S/F42A/Y45A/A73T
DKTHTCPPCPAPEAAGGPSVFLFPPKPKDTLMISRTPEVTCVVVDVSHEDPEVKENWYVDGVEVHNAK
TKPREEQYNSTYRVVSVLTVLHQDWLNGKEYKCKVSNKALPAPIEKTISKAKGQPREPQVYTLPPSRE
EMTKNQVSLTCLVKGFYPSDIAVEWESNGQPENNYKTTPPVLDSDGSFFLYSKLTVDKSRWQQGNVES

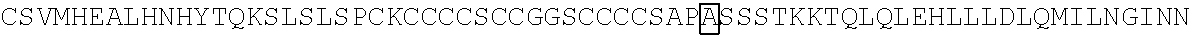

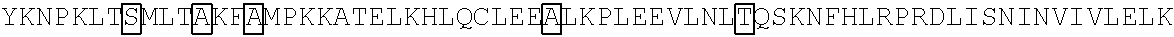

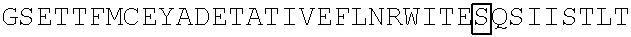

SEQ ID NO: 136- IgG1FC (with LALA and Knob)-IL-2-T3A/C125S-
K35N/R38S/F42A/Y45A/A73T
DKTHTCPPCPAPEAAGGPSVELFPPKPKDTLMISRTPEVTCVVVDVSHEDPEVKENWYVDGVEVHNAK
TKPREEQYNSTYRVVSVLTVLHQDWLNGKEYKCKVSNKALPAPIEKTISKAKGQPREPQVYTLPPSRE
EMTKNQVSLTCLVKGFYPSDIAVEWESNGQPENNYKTTPPVLDSDGSFFLYSKLTVDKSRWQQGNVES

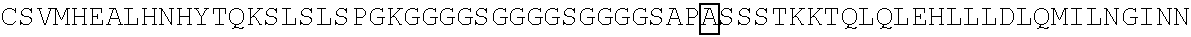

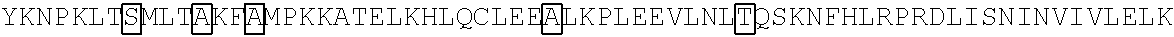

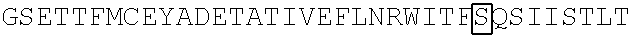

SEQ ID NO: 137- IgG1FC (with LALA and Knob)-IL-2-T3A/C125S-
R38S/F42R/Y45A/E62A no linker
DKTHTCPPCPAPEAAGGPSVELFPPKPKDTLMISRTPEVTCVVVDVSHEDPEVKENWYVDGVEVHNAK
TKPREEQYNSTYRVVSVLTVLHQDWLNGKEYKCKVSNKALPAPIEKTISKAKGQPREPQVYTLPPSRE
EMTKNQVSLTCLVKGFYPSDIAVEWESNGQPENNYKTTPPVLDSDGSFFLYSKLTVDKSRWQQGNVES

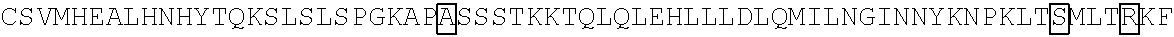

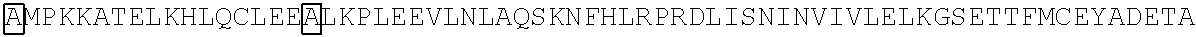

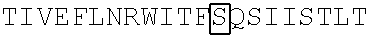

SEQ ID NO: 138- HSA-IL-2-T3A/C125S-R38S/F42A/Y45A/E62A/N88A/Q126H
with cleavable linker
DAHKSEVAHRFKDLGEENFKALVLIAFAQYLQQCPFEDHVKLVNEVTEFAKTCVADESAENCDKSLHT
LFGDKLCTVATLRETYGEMADCCAKQEPERNECFLQHKDDNPNLPRLVRPEVDVMCTAFHDNEETFLK
KYLYEIARRHPYFYAPELLFFAKRYKAAFTECCQAADKAACLLPKLDELRDEGKASSAKQRLKCASLQ
KFGERAFKAWAVARLSQRFPKAEFAEVSKLVTDLTKVHTECCHGDLLECADDRADLAKYICENQDSIS
SKLKECCEKPLLEKSHCIAEVENDEMPADLPSLAADFVESKDVCKNYAEAKDVELGMELYEYARRHPD
YSVVLLLRLAKTYETTLEKCCAAADPHECYAKVFDEFKPLVEEPQNLIKQNCELFEQLGEYKFQNALL
VRYTKKVPQVSTPTLVEVSRNLGKVGSKCCKHPEAKRMPCAEDYLSVVLNQLCVLHEKTPVSDRVTKC
CTESLVNRRPCFSALEVDETYVPKEFNAETFTFHADICTLSEKERQIKKQTALVELVKHKPKATKEQL
KAVMDDFAAFVEKCCKADDKETCFAEEGPKLVAASQAALGLGGGGSGGGGSLSGRSDNHGGSGGSAPA
SSSTKKTQLQLEHLLLDLQMILNGINNYKNPKLTSMLTAKFAMPKKATELKHLQCLEEALKPLEEVLN

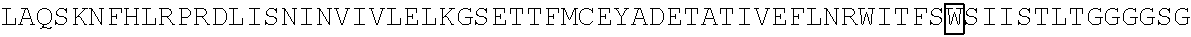

GGGSGGGGSDKTHTCPPCPAPEAAGGPSVELFPPKPKDTLMISRTPEVTCVVVDVSHEDPEVKENWYV
DGVEVHNAKTKPREEQYNSTYRVVSVLTVLHQDWLNGKEYKCKVSNKALPAPIEKTISKAKGQPREPQ
VYTLPPSREEMTKNQVSLTCLVKGFYPSDIAVEWESNGQPENNYKTTPPVLDSDGSFFLYSKLTVDKS
RWQQGNVFSCSVMHEALHNHYTQKSLSLSPGK
SEQ ID NO: 139- IgG1FC (with LALA and Knob)-IL-2-T3A/C125S-
F42A/Y45A/E62A/N88E
DKTHTCPPCPAPEAAGGPSVFLFPPKPKDTLMISRTPEVTCVVVDVSHEDPEVKENWYVDGVEVHNAK
TKPREEQYNSTYRVVSVLTVLHQDWLNGKEYKCKVSNKALPAPIEKTISKAKGQPREPQVYTLPPSRE
EMTKNQVSLTCLVKGFYPSDIAVEWESNGQPENNYKTTPPVLDSDGSFFLYSKLTVDKSRWQQGNVES

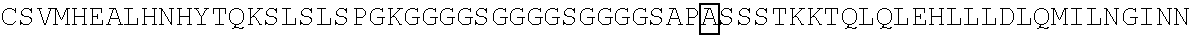

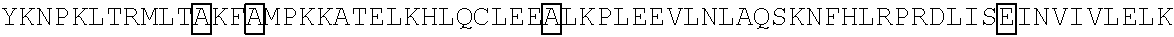

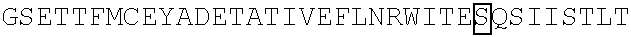

SEQ ID NO: 140- IL-2-T3A/C125S-R38S/F42A/Y45A/E62A/Q126W-IgG4 Fc
APASSSTKKTQLQLEHLLLDLQMILNGINNYKNPKLTSMLTAKFAMPKKATELKHLQCLEEALKPLEE

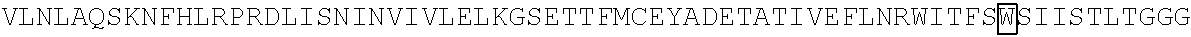

GSGGGGSGGGGSDKTHTCPPCPAPEAAGGPSVELFPPKPKDTLMISRTPEVTCVVVDVSHEDPEVKEN
WYVDGVEVHNAKTKPREEQYNSTYRVVSVLTVLHQDWLNGKEYKCKVSNKALPAPIEKTISKAKGQPR
EPQVYTLPPSREEMTKNQVSLYCLVKGFYPSDIAVEWESNGQPENNYKTTPPVLDSDGSFFLYSKLTV
DKSRWQQGNVFSCSVMHEALHNHYTQKSLSLSPGK
SEQ ID NO: 141- IL-2-T3A/C125S-R38S/F42A/Y45A/E62A/Q126W-IgG1 Fc
homodimer
APASSSTKKTQLQLEHLLLDLQMILNGINNYKNPKLTSMLTAKFAMPKKATELKHLQCLEEALKPLEE

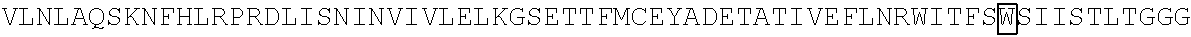

GSGGGGGGGGSDKTHTCPPCPAPEAAGGPSVFLFPPKPKDTLMISRTPEVTCVVVDVSHEDPEVKEN
WYVDGVEVHNAKTKPREEQYNSTYRVVSVLTVLHQDWLNGKEYKCKVSNKALPAPIEKTISKAKGQPR
EPQVYTLPPSREEMTKNQVSLTCLVKGFYPSDIAVEWESNGQPENNYKTTPPVLDSDGSFFLYSKLTV
DKSRWQQGNVFSCSVMHEALHNHYTQKSLSLSPGK
SEQ ID NO: 142- IgG4 Fc - IL-2-T3A/C125S-R38S/F42A/Y45A/E62A/Q126A
AESKYGPPCPPCPAPEAAGGPSVFLFPPKPKDQLMISRTPEVTCVVVDVSQEDP
EVQFNWYVDGVEVHNAKTKPREEQFNSTYRVVSVLTVLHQDWLNGKEYKC KVSNKGLPSS
IEKTISKAKGQPREPQVYTLPPSQEEMTKNQVSLTCLVKG
FYPSDIAVEWESNGQPENNYKTTPPVLDSDGSFFLYSRLTVDKSRWQEGN

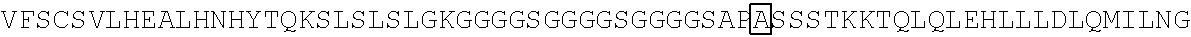

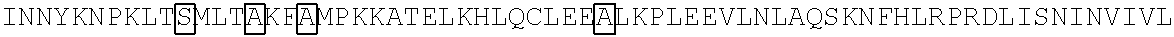

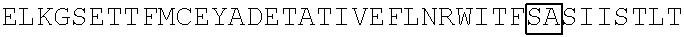

SEQ ID NO: 143- IgG1Fc with YTE/LALA/IL-2 N88A/Q126H/beta/gamma
DKTHTCPPCP APEAAGGPSV FLFPPKPKDT LYITREPEVT CVVVDVSHED PEVKENWYVD
GVEVHNAKTK PREEQYNSTY RVVSVLTVLH QDWLNGKEYK CKVSNKALPA PIEKTISKAK
GQPREPQVYT LPPSRDELTK NQVSLTCLVK GFYPSDIAVE WESNGQPENN YKTTPPVLDS
DGSFFLYSKL TVDKSRWQQG NVFSCSVMHE ALHNHYTQKS LSLSPGKGGG GSGGGGSGGG
GGSAPASSST KKTQLQLEHL LLDLQMILNG INNYKNPKLT SMLTAKFAMP KKATELKHLQ
CLEEALKPLE EVLNLAQSKN FHLRPRDLIS AINVIVLELK GSETTEMCEY ADETATIVEF
LNRWITFSHS IISTLTGGGG STARGPSFKG GGGSAVNGTS QFTCFYNSRA NISCVWSQDG
ALQDTSCQVH AWPDRRRWNQ TCELLPVSQA SWACNLILGA PDSQKLTTVD IVTLRVLCRE
GVRWRVMAIQ DFKPFENLRL MAPISLQVVH VETHRCNISW EISQASHYFE RHLEFEARTL
SPGHTWEEAP LLTLKQKQEW ICLETLTPDT QYEFQVRVKP LQGEFTTWSP WSQPLAFRTK
PAALGKDTGG GGSGGGGSLS GRSDNHGGGG SGGGGSLNTT ILTPNGNEDT TADFELTTMP
TDSLSVSTLP LPEVQCEVEN VEYMNCTWNS SSEPQPTNLT LHYWYKNSDN DKVQKCSHYL
FSEEITSGCQ LQKKEIHLYQ TFVVQLQDPR EPRRQATQML KLQNLVIPWA PENLTLHKLS
ESQLELNWNN RELNHCLEHL VQYRTDWDHS WTEQSVDYRH KESLPSVDGQ KRYTERVRSR
FNPLCGSAQH WSEWSHPIHW GSNTSKENPF LFALEA
SEQ ID NO: 144- IgG1Fc with YTE/LALA/IL-2/Hole

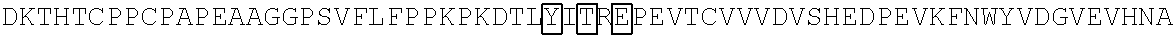

KTKPREEQYNSTYRVVSVLTVLHQDWLNGKEYKCKVSNKALPAPIEKTISKAKGQPREPQVYTLPPSR
DELTKNQVSLTCLVKGFYPSDIAVEWESNGQPENNYKTTPPVLDSDGSFFLTSKLTVDKSRWQQGNVE
SCSVMHEALHNHYTQKSLSLSPGK

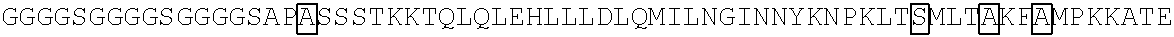

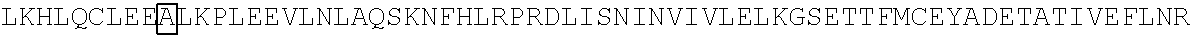

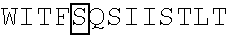

SEQ ID NO: 145- IgG1Fc with YTE/LALA/Knob/beta

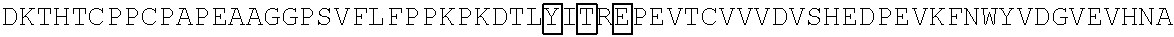

KTKPREEQYNSTYRVVSVLTVLHQDWLNGKEYKCKVSNKALPAPIEKTISKAKGQPREPQVYTLPPSR
DELTKNQVSLYCLVKGFYPSDIAVEWESNGQPENNYKTTPPVLDSDGSFFLYSKLTVDKSRWQQGNVE
SCSVMHEALHNHYTQKSLSLSPGKGGGGSTARGPSFKGGGGSAVNGTSQFTCFYNSRANISCVWSQDG
ALQDTSCQVHAWPDRRRWNQTCELLPVSQASWACNLILGAPDSQKLTTVDIVTLRVLCREGVRWRVMA
IQDFKPFENLRLMAPISLQVVHVETHRCNISWEISQASHYFERHLEFEARTLSPGHTWEEAPLLTLKQ
KQEWICLETLTPDTQYEFQVRVKPLQGEFTTWSPWSQPLAFRTKPAALGKDT
SEQ ID NO: 146- IgG1Fc with YTE/LALA/IL-20126W/Hole

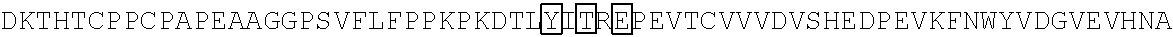

KTKPREEQYNSTYRVVSVLTVLHQDWLNGKEYKCKVSNKALPAPIEKTI SKAKGQPREPQVYTLPPSR
DELTKNQVSLTCLVKGFYPSDIAVEWESNGQPENNYKTTPPVLDSDGSFFLTSKLTVDKSRWQQGNVE

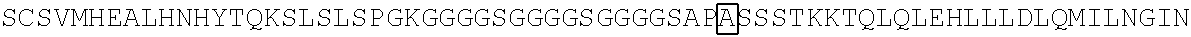

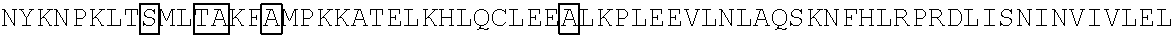

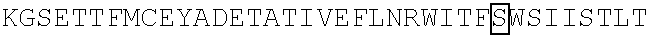

SEQ ID NO: 147- IgG1Fc with YTE/LALA/IL-2/Knob/Beta/gamma

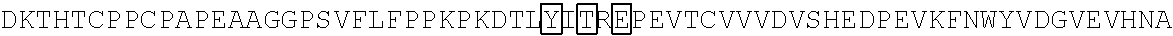

KTKPREEQYNSTYRVVSVLTVLHQDWLNGKEYKCKVSNKALPAPIEKTISKAKGQPREPQVYTLPPSR
DELTKNQVSLYCLVKGFYPSDIAVEWESNGQPENNYKTTPPVLDSDGSFFLYSKLTVDKSRWQQGNVE
SCSVMHEALHNHYTQKSLSLSPGKGGGGSTARGPSFKGGGGSAVNGTSQFTCFYNSRANISCVWSQDG
ALQDTSCQVHAWPDRRRWNQTCELLPVSQASWACNLILGAPDSQKLTTVDIVTLRVLCREGVRWRVMA
IQDFKPFENLRLMAPISLQVVHVETHRCNISWEISQASHYFERHLEFEARTLSPGHTWEEAPLLTLKQ
KQEWICLETLTPDTQYEFQVRVKPLQGEFTTWSPWSQPLAFRTKPAALGKDTGGGGSGGGGS
LSGRSDNHGGGGSGGGGS LNTTILTPNG NEDTTADFFL TTMPTDSLSV STLPLPEVQC
FVENVEYMNC TWNSSSEPQP TNLTLHYWYK NSDNDKVQKC SHYLFSEEIT SGCQLQKKEI
HLYQTFVVQL QDPREPRRQA TQMLKLQNLV IPWAPENLTL HKLSESQLEL NWNNRELNHC
LEHLVQYRTD WDHSWTEQSV DYRHKESLPS VDGQKRYTER VRSRENPLCG SAQHWSEWSH
PIHWGSNTSK ENPFLFALEA
SEQ ID NO: 148- trastuzumab light chain
DIQMTQSPSSLSASVGDRVTITCRASQDVNTAVAWYQQKPGKAPKLLIYSASFLYSGVPSRFSGSRSG
TDFTLTISSLQPEDFATYYCQQHYTTPPTFGQGTKVEIKRTVAAPSVFIFPPSDEQLKSGTASVVCLL
NNFYPREAKVQWKVDNALQSGNSQESVTEQDSKDSTYSLSSTLTLSKADYEKHKVYACEVTHQGLSSP
VTKSENRGEC
SEQ ID NO: 149- trastuzumab heavy chain
EVQLVESGGGLVQPGGSLRLSCAASGENIKDTYIHWVRQAPGKGLEWVARIYPTNGYTRYADSVKGRE
TISADTSKNTAYLQMNSLRAEDTAVYYCSRWGGDGFYAMDYWGQGTLVTVSSASTKGPSVFPLAPSSK
STSGGTAALGCLVKDYFPEPVTVSWNSGALTSGVHTFPAVLQSSGLYSLSSVVTVPSSSLGTQTYICN
VNHKPSNTKVDKKVEPPKSCDKTHTCPPCPAPELLGGPSVFLFPPKPKDTLMISRTPEVTCVVVDVSH
EDPEVKFNWYVDGVEVHNAKTKPREEQYNSTYRVVSVLTVLHQDWLNGKEYKCKVSNKALPAPIEKTI
SKAKGQPREPQVYTLPPSRDELTKNQVSLTCLVKGFYPSDIAVEWESNGQPENNYKTTPPVLDSDGSF
FLYSKLTVDKSRWQQGNVFSCSVMHEALHNHYTQKSLSLSPGK
SEQ ID NO: 150- rituximab light chain
QIVLSQSPAILSASPGEKVTMTCRASSSVSYIHWFQQKPGSSPKPWIYATSNLASGVPVRFSGSGSGT
SYSLTISRVEAEDAATYYCQQWTSNPPTFGGGTKLEIKRTVAAPSVFIFPPSDEQLKSGTASVVCLLN
NFYPREAKVQWKVDNALQSGNSQESVTEQDSKDSTYSLSSTLTLSKADYEKHKVYACEVTHQGLSSPV
TKSENRGEC
SEQ ID NO: 151- rituximab heavy chain
QVQLQQPGAELVKPGASVKMSCKASGYTFTSYNMHWVKQTPGRGLEWIGAIYPGNGDTSYNQKEKGKA
TLTADKSSSTAYMQLSSLTSEDSAVYYCARSTYYGGDWYFNVWGAGTTVTVSAASTKGPSVEPLAPSS
KSTSGGTAALGCLVKDYFPEPVTVSWNSGALTSGVHTFPAVLQSSGLYSLSSVVTVPSSSLGTQTYIC
NVNHKPSNTKVDKKAEPKSCDKTHTCPPCPAPELLGGPSVFLFPPKPKDTLMISRTPEVTCVVVDVSH
EDPEVKENWYVDGVEVHNAKTKPREEQYNSTYRVVSVLTVLHQDWLNGKEYKCKVSNKALPAPIEKTI
SKAKGQPREPQVYTLPPSRDELTKNQVSLTCLVKGFYPSDIAVEWESNGQPENNYKTTPPVLDSDGSF
FLYSKLTVDKSRWQQGNVFSCSVMHEALHNHYTQKSLSLSPGK
SEQ ID NO: 152- brentuximab light chain
DIVLTQSPASLAVSLGQRATISCKASQSVDFDGDSYMNWYQQKPGQPPKVLIYAASNLESGIPARFSG
SGSGTDETLNIHPVEEEDAATYYCQQSNEDPWTFGGGTKLEIKRTVAAPSVFIFPPSDEQLKSGTASV
VCLLNNFYPREAKVQWKVDNALQSGNSQESVTEQDSKDSTYSLSSTLTLSKADYEKHKVYACEVTHQG
LSSPVTKSENRGEC
SEQ ID NO: 153- brentuximab heavy chain
QIQLQQSGPEVVKPGASVKISCKASGYTFTDYYITWVKQKPGQGLEWIGWIYPGSGNTKYNEKFKGKA
TLTVDTSSSTAFMQLSSLTSEDTAVYFCANYGNYWFAYWGQGTQVTVSAASTKGPSVFPLAPSSKSTS
GGTAALGCLVKDYFPEPVTVSWNSGALTSGVHTFPAVLQSSGLYSLSSVVTVPSSSLGTQTYICNVNH
KPSNTKVDKKVEPKSCDKTHTCPPCPAPELLGGPSVELFPPKPKDTLMISRTPEVTCVVVDVSHEDPE
VKFNWYVDGVEVHNAKTKPREEQYNSTYRVVSVLTVLHQDWLNGKEYKCKVSNKALPAPIEKTISKAK
GQPREPQVYTLPPSRDELTKNQVSLTCLVKGFYPSDIAVEWESNGQPENNYKTTPPVLDSDGSFFLYS
KLTVDKSRWQQGNVFSCSVMHEALHNHYTQKSLSLSPG
SEQ ID NO: 154- cetuximab light chain
DILLTQSPVILSVSPGERVSFSCRASQSIGTNIHWYQQRINGSPRLLIKYASESISGIPSRFSGSGSG
TDFTLSINSVESEDIADYYCQQNNNWPTTFGAGTKLELKRTVAAPSVFIFPPSDEQLKSGTASVVCLL
NNFYPREAKVQWKVDNALQSGNSQESVTEQDSKDSTYSLSSTLTLSKADYEKHKVYACEVTHQGLSSP
VTKSENRGEC
SEQ ID NO: 155- cetuximab heavy chain
QVQLKQSGPGLVQPSQSLSITCTVSGFSLTNYGVHWVRQSPGKGLEWLGVIWSGGNTDYNTPFTSRLS
INKDNSKSQVFFKMNSLQSNDTAIYYCARALTYYDYEFAYWGQGTLVTVSAASTKGPSVFPLAPSSKS
TSGGTAALGCLVKDYFPEPVTVSWNSGALTSGVHTFPAVLQSSGLYSLSSVVTVPSSSLGTQTYICNV
NHKPSNTKVDKKVEPKSCDKTHTCPPCPAPELLGGPSVELFPPKPKDTLMISRTPEVTCVVVDVSHED
PEVKFNWYVDGVEVHNAKTKPREEQYNSTYRVVSVLTVLHQDWLNGKEYKCKVSNKALPAPIEKTISK
AKGQPREPQVYTLPPSRDELTKNQVSLTCLVKGFYPSDIAVEWESNGQPENNYKTTPPVLDSDGSFEL
YSKLTVDKSRWQQGNVFSCSVMHEALHNHYTQKSLSLSPGK
SEQ ID NO: 156- panitumumab light chain
DIQMTQSPSSLSASVGDRVTITCQASQDISNYLNWYQQKPGKAPKLLIYDASNLETGVPSRFSGSGSG
TDFTFTISSLQPEDIATYFCQHFDHLPLAFGGGTKVEIKRTVAAPSVFIFPPSDEQLKSGTASVVCLL
NNFYPREAKVQWKVDNALQSGNSQESVTEQDSKDSTYSLSSTLTLSKADYEKHKVYACEVTHQGLSSP
VTKSENRGEC
SEQ ID NO: 157- panitumumab heavy chain
GHIYYSGNTNYNPSLKSRLTISIDTSKTQFSLKLSSVTAADTAIYYCVRDRVTGAFDIWGQGTMVTVS
SASTKGPSVFPLAPCSRSTSESTAALGCLVKDYFPEPVTVSWNSGALTSGVHTFPAVLQSSGLYSLSS
VVTVPSSNFGTQTYTCNVDHKPSNTKVDERKCCVECPAGPSVFLFPPKPKDTLMISRTPEVTCVVVDV
SHEDPEVQFNWYVDGVEVHNAKTKPREEQFNSTFRVVSVLTVVHQDWLNGKEYKCKVSNKGLPAPIEK
TISKTKGQPREPQVYTLPPSREEMTKNQVSLTCLVKGFYPSDIAVEWESNGQPENNYKTTPPMLDSDG
SFFLYSKLTVDKSRWQQGNVFSCSVMHEALHNHYTQKSLSLSPGK
SEQ ID NO: 158- anti-c-MET antibody light chain
DIVMTQAAPSVPVTPGESVSISCRSSKSLLHSNGNTYLYWFLQRPGQSPQVLIYRMSNLASGVPDRES
GSGSGTAFTLRIRRVEAEDVGVYYCMQNLEYPFTFGGGTKLEIKRTVAAPSVFIFPPSDEQLKSGTAS
VVCLLNNFYPREAKVQWKVDNALQSGNSQESVTEQDSKDSTYSLSSTLTLSKADYEKHKVYACEVTHQ
GLSSPVTKSENRGEC
SEQ ID NO: 159- anti-c-MET antibody heavy chain
QVQLQQSGPELVKSGASVKMSCKASGNTLKDDHVHWVKQRPGQGLEWIGWIYPGGGRTRYNEKFKGKT
TLTADKPSSTVNMLLSSLTSEDSAIYFCTNLVFDVWGAGTTVTVSSASTKGPSVFPLAPSSKSTSGGT
AALGCLVKDYFPEPVTVSWNSGALTSGVHTFPAVLQSSGLYSLSSVVTVPSSSLGTQTYICNVNHKPS
NTKVDKKVEPKSCDKTHTCPPCPAPELLGGPSVFLFPPKPKDTLMISRTPEVTCVVVDVSHEDPEVKE
NWYVDGVEVHNAKTKPREEQYNSTYRVVSVLTVLHQDWLNGKEYKCKVSNKALPAPIEKTISKAKGQP
REPQVYTLPPSREEMTKNQVSLTCLVKGFYPSDIAVEWESNGQPENNYKTTPPVLDSDGSFFLYSKLT
VDKSRWQQGNVFSCSVMHEALHNHYTQKSLSLSPGK
SEQ ID NO: 160- anti-GPC3 antibody light chain
DVVMTQSPLSLPVTPGEPASISCRSSQSLVHSNANTYLHWYLQKPGQSPQLLIYKVSNRFSGVPDRES
GSGSGTDFTLKISRVEAEDVGVYYCSQNTHVPPTFGQGTKLEIKRTVAAPSVFIFPPSDEQLKSGTAS
VVCLLNNFYPREAKVQWKVDNALQSGNSQESVTEQDSKDSTYSLSSTLTLSKADYEKHKVYACEVTHQ
GLSSPVTKSENRGEC
SEQ ID NO: 161- anti-GPC3 antibody heavy chain
QVQLVQSGAEVKKPGASVKVSCKASGYTFTDYEMHWVRQAPGQGLEWMGALDPKTGDTAYSQKFKGRV
TLTADKSTSTAYMELSSLTSEDTAVYYCTRFYSYTYWGQGTLVTVSSASTKGPSVFPLAPSSKSTSGG
TAALGCLVKDYFPEPVTVSWNSGALTSGVHTFPAVLQSSGLYSLSSVVTVPSSSLGTQTYICNVNHKP
SNTKVDKKVEPKSCDKTHTCPPCPAPELLGGPSVFLFPPKPKDTLMISRTPEVTCVVVDVSHEDPEVK
FNWYVDGVEVHNAKTKPREEQYNSTYRVVSVLTVLHQDWLNGKEYKCKVSNKALPAPIEKTISKAKGQ
PREPQVYTLPPSREEMTKNQVSLTCLVKGFYPSDIAVEWESNGQPENNYKTTPPVLDSDGSFFLYSKL
TVDKSRWQQGNVFSCSVMHEALHNHYTQKSLSLSPGK
SEQ ID NO: 162- anti-Claudin 18.2 antibody light chain
DIVMTQSPSSLTVTAGEKVTMSCKSSQSLLNSGNQKNYLTWYQQKPGQPPKLLIYWASTRESGVPDRF
TGSGSGTDFTLTISSVQAEDLAVYYCQNDYSYPFTFGSGTKLEIKRTVAAPSVFIFPPSDEQLKSGTA
SVVCLLNNFYPREAKVQWKVDNALQSGNSQESVTEQDSKDSTYSLSSTLTLSKADYEKHKVYACEVTH
QGLSSPVTKSENRGEC
SEQ ID NO: 163- anti-Claudin 18.2 antibody heavy chain
QVQLQQPGAELVRPGASVKLSCKASGYTFTSYWINWVKQRPGQGLEWIGNIYPSDSYTNYNQKFKDKA
TLTVDKSSSTAYMQLSSPTSEDSAVYYCTRSWRGNSFDYWGQGTTLTVSSASTKGPSVFPLAPSSKST
SGGTAALGCLVKDYFPEPVTVSWNSGALTSGVHTFPAVLQSSGLYSLSSVVTVPSSSLGTQTYICNVN
HKPSNTKVDKKVEPKSCDKTHTCPPCPAPELLGGPSVELFPPKPKDTLMISRTPEVTCVVVDVSHEDP
EVKFNWYVDGVEVHNAKTKPREEQYNSTYRVVSVLTVLHQDWLNGKEYKCKVSNKALPAPIEKTISKA
KGQPREPQVYTLPPSRDELTKNQVSLTCLVKGFYPSDIAVEWESNGQPENNYKTTPPVLDSDGSFFLY
SKLTVDKSRWQQGNVFSCSVMHEALHNHYTQKSLSLSPGK
SEQ ID NO: 164- anti-Trop-2 antibody light chain CDR1
KASQDVSIAVA
SEQ ID NO: 165- anti-Trop-2 antibody light chain CDR2
SASYRYT
SEQ ID NO: 166- anti-Trop-2 antibody light chain CDR3
QQHYITPLT
SEQ ID NO: 167- anti-Trop-2 antibody heavy chain CDR1
NYGMN
SEQ ID NO: 168- anti-Trop-2 antibody heavy chain CDR2
WINTYTGEPTYTDDFKG
SEQ ID NO: 169- anti-Trop-2 antibody heavy chain CDR3
GGFGSSYWYFDV
SEQ ID NO: 170- anti-mesothelin antibody light chain CDR1
SASSSVSYMH
SEQ ID NO: 171- anti-mesothelin antibody light chain CDR2
DTSKLAS
SEQ ID NO: 172- anti-mesothelin antibody light chain CDR3
QQWSGYPLT
SEQ ID NO: 173- anti-mesothelin antibody heavy chain CDR1
GYTMN
SEQ ID NO: 174- anti-mesothelin antibody heavy chain CDR2
LITPYNGASSYNQKFRG
SEQ ID NO: 175- anti-mesothelin antibody heavy chain CDR3
GGYDGRGFDY
SEQ ID NO: 176- Light Chain variable domain of PR1A3.
GDIVMTQSQRFMSTSVGDRVSVTCKASQNVGTNVAWYQQKPGQSPKALIYSASYRYSGVPDRETGSG-
SGTDFTLTISNVQSEDLAEYFCHQYYTYPLFTFGSGTKLEMKR
SEQ ID NO: 177- Heavy Chain variable domain of PR1A3.
QVKLQQSGPELKKPGETVKISCKASGYTFTVFGMNWVKQAPGKGLKWMGWINTKTGEATYVEEFKGRF
AFSLETSATTAYLQINNLKNEDTAKYFCARWDFYDYVEAMDYWGQGTTVTVSS
SEQ ID NO: 178- Humanized Light Chain variable domain PR1A3.
DIQMTQSPSSLSASVGDRVTITCKASQNVGTNVAWYQQKPGKAPKLLIYSASYRYSGVPSRESGSGSG
TDFTFTISSLQPEDIATYYCHQYYTYPLFTFGQGTKVEIKR
SEQ ID NO: 179- Humanized Heavy Chain variable domain of PR1A3.
QVQLVQSGSELKKPGASVKVSCKASGYTFTVFGMNWVRQAPGQGLEWMGWINTKTGEATYVEEFKGREF
VESLDTSVSTAYLQISSLKADDTAVYYCARWDFYDYVEAMDYWGQGTTVTVSS
SEQ ID NO: 180- Anti-FAP version 1 LC (protein sequence)

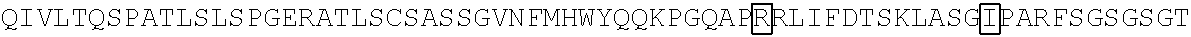

DYTLTISSLEPEDFAVYYCQQWSFNPPTFGQGTKVEIKRTVAAPSVFIFPPSDEQLKSGTASVVCLLN
NFYPREAKVQWKVDNALQSGNSQESVTEQDSKDSTYSLSSTLTLSKADYEKHKVYACEVTHQGLSSPV
TKSFNRGEC
SEQ ID NO: 181-Anti-FAP LC version 2 (protein sequence)

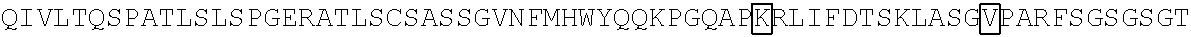

DYTLTISSLEPEDFAVYYCQQWSFNPPTFGQGTKVEIKRTVAAPSVFIFPPSDEQLKSGTASVVCLLN
NFYPREAKVQWKVDNALQSGNSQESVTEQDSKDSTYSLSSTLTLSKADYEKHKVYACEVTHQGLSSPV
TKSENRGEC
SEQ ID NO: 182- Anti-FAP VH (protein sequence)
QVQLVQSGAE VKKPGASVKV SCKASGYTFT NNGINWLRQA PGQGLEWMGE
IYPRSTNTLYAQKFQGRVTITADRSSNTAYMELSSLRSEDTAVYFCARTLTAPFAFWGQGTLVTVSSA
STKGPSVFPLAPSSKSTSGGTAALGCLVKDYFPEPVTVSWNSGALTSGVHTFPAVLQSSGLYSLSSVV
TVPSSSLGTQTYICNVNHKPSNTKVDKKVEPKSCDKTHTCPPCPAPELLGGPSVFLFPPKPKDTLMIS
RTPEVTCVVVDVSHEDPEVKFNWYVDGVEVHNAKTKPREEQYNSTYRVVSVLTVLHQDWLNGKEYKCK

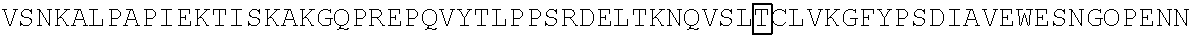

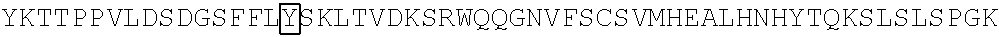

SEQ ID NO: 183- Humanized Light Chain variable domain of FAPalpha
antibody BIBH1
DIVMTQSPDS LAVSLGERAT INCKSSQSLL YSRNQKNYLA WYQQKPGQPP KLLIFWASTR
ESGVPDRESG SGFGTDFTLT ISSLQAEDVA VYYCQQYFSY PLTFGQGTKV EIK
SEQ ID NO: 184- Humanized Heavy Chain variable domain FAPalpha
antibody BIBH1
QVQLVQSGAE VKKPGASVK VSCKTSRYTFT EYTIHWVRQA PGQRLEWIGG INPNNGIPNY
NQKFKGRVTI TVDTSASTAY MELSSLRSED TAVYYCARRR IAYGYDEGHA MDYWGQGTLV
TVSS
SEQ ID NO: 185- Humanized H8 anti-5T4 version 1 VH (protein
sequence)
QVQLVQSGAEVKKPGASVKVSCKASGYSFTGYYMHWVKQSPGQGLEWIGRINPNNGV
TLYNQKFKDRVTMTRDTSISTAYMELSRLRSDDTAVYYCARSTMITNYVMDYWGQGT
LWTVSS
SEQ ID NO: 186- Humanized H8 anti-5T4 VH version 2 (protein
sequence)
QVQLVQSGAEVKKPGASVKVSCKASGYSFTGYYMHWVRQAPGQGLEWMGRINPNNGVTLYNQKFKDRV
TMTRDTSISTAYMELSRLRSDDTAVYYCARSTMITNYVMDYWGQGTLVTVSS
SEQ ID NO: 187- Humanized H8 anti-5T4 version 1 VL (protein
sequence)
DIVMTQSPDSLAVSLGERATINCKASQSVSNDVAWYQQKPGQSPKLLISYTSSRYAG
VPDRESGSGSGTDFTLTISSLQAEDVAVYFCQQDYNSPPTFGGGTKLEIK
SEQ ID NO: 188- Humanized H8 anti-5T4 VL version 2 (protein
sequence)
DIVMTQSPDSLAVSLGERAT INCKASQSVSNDVAWYQQKPGQPPKLLIYYTSSRYAG
VPDRFSGSGSGTDFTLTISSLQAEDVAVYYCQQDYNSPPTFGGGTKLEIK
SEQ ID NO: 189- Anti-PDL1 atezolizumab LC
DIQMTQSPSSLSASVGDRVTITCRASQDVSTAVAWYQQKPGKAPKLLIYSASFLYSGVPS
RFSGSGSGTDFTLTISSLQPEDFATYYCQQYLYHPATFGQGTKVEIKRTVAAPSVFIFPP
SDEQLKSGTASVVCLLNNFYPREAKVQWKVDNALQSGNSQESVTEQDSKDSTYSLSSTLTLSKADYEK
HKVYACEVTHQGLSSPVTKSFNRGEC
SEQ ID NO: 190- Anti-PDL1 atezolizumab HC (protein sequence)
EVQLVESGGGLVQPGGSLRLSCAASGFTFSDSWIHWVRQAPGKGLEWVAWISPYGGSTYYADSVKGRE
TISADTSKNTAYLQMNSLRAEDTAVYYCARRHWPGGFDYWGQGTLVTVSSASTKGPSVFPLAPSSKST
SGGTAALGCLVKDYFPEPVTVSWNSGALTSGVHTFPAVLQSSGLYSLSSVVTVPSSSLGTQTYICNVN
HKPSNTKVDKKVEPKSCDKTHTCPPCPAPELLGGPSVFLFPPKPKDTLMISRTPEVTCVVVDVSHEDP
EVKFNWYVDGVEVHNAKTKPREEQYASTYRVVSVLTVLHQDWLNGKEYKCKVSNKALPAPIEKTISKA
KGQPREPQVYTLPPSREEMTKNQVSLTCLVKGFYPSDIAVEWESNGQPENNYKTTPPVLDSDGSFFLY
SKLTVDKSRWQQGNVFSCSVMHEALHNHYTQKSLSLSPGK
SEQ ID NO: 191- Anti-PDL1 antibody atezolizumab HC (protein
sequence) fused with IL-2-T3A/C125S-R38S/F42A/Y45A/E62A, wherein Fc
has mutation T366Y
EVQLVESGGGLVQPGGSLRLSCAASGFTFSDSWIHWVRQAPGKGLEWVAWISPYGGSTYYADSVKGRF
TISADTSKNTAYLQMNSLRAEDTAVYYCARRHWPGGFDYWGQGTLVTVSSASTKGPSVFPLAPSSKST
SGGTAALGCLVKDYFPEPVTVSWNSGALTSGVHTFPAVLQSSGLYSLSSVVTVPSSSLGTQTYICNVN
HKPSNTKVDKKVEPKSCDKTHTCPPCPAPELLGGPSVELFPPKPKDTLMISRTPEVTCVVVDVSHEDP
EVKFNWYVDGVEVHNAKTKPREEQYASTYRVVSVLTVLHQDWLNGKEYKCKVSNKALPAPIEKTISKA
KGQPREPQVYTLPPSREEMTKNQVSLYCLVKGFYPSDIAVEWESNGQPENNYKTTPPVLDSDGSFFLY
SKLTVDKSRWQQGNVFSCSVMHEALHNHYTQKSLSLSPGKGGGSGGGSGGGSAPASSSTKKTQLQLEH
LLLDLQMILNGINNYKNPKLTSMLTAKFAMPKKATELKHLQCLEEALKPLEEVLNLAQSKNFHLRPRD
LISNINVIVLELKGSETTEMCEYADETATIVEFLNRWITFSQSIISTLT
SEQ ID NO: 192- Anti-PDL1 atezolizumab HC with (Y407T) with 2xbeta
(hole)
EVQLVESGGGLVQPGGSLRLSCAASGFTFSDSWIHWVRQAPGKGLEWVAWISPYGGSTYYADSVKGRF
TISADTSKNTAYLQMNSLRAEDTAVYYCARRHWPGGFDYWGQGTLVTVSSASTKGPSVFPLAPSSKST
SGGTAALGCLVKDYFPEPVTVSWNSGALTSGVHTFPAVLQSSGLYSLSSVVTVPSSSLGTQTYICNVN
HKPSNTKVDKKVEPKSCDKTHTCPPCPAPELLGGPSVFLFPPKPKDTLMISRTPEVTCVVVDVSHEDP
EVKFNWYVDGVEVHNAKTKPREEQYASTYRVVSVLTVLHQDWLNGKEYKCKVSNKALPAPIEKTISKA
KGQPREPQVYTLPPSREEMTKNQVSLTCLVKGFYPSDIAVEWESNGQPENNYKTTPPVLDSDGSFELT
SKLTVDKSRWQQGNVFSCSVMHEALHNHYTQKSLSLSPGKGGGGSGGGGSGGGGISSGLLSSGGSGGS
LSGRSDNHGGGGSAVNGTSQFTCFYNSRANISCVWSQDGALQDTSCQVHAWPDRRRWNQTCELLPVSQ
ASWACNLILGAPDSQKLTTVDIVTLRVLCREGVRWRVMAIQDFKPFENLRLMAPISLQVVHVETHRCN
ISWEISQASHYFERHLEFEARTLSPGHTWEEAPLLTLKQKQEWICLETLTPDTQYEFQVRVKPLQGEF
TTWSPWSQPLAFRTKPAALGKDTGGGGSGGGGSGGGGSISSGLLSSGGSGGSLSGRSDNHGGGGSGGG
GSAVNGTSQFTCFYNSRANISCVWSQDGALQDTSCQVHAWPDRRRWNQTCELLPVSQASWACNLILGA
PDSQKLTTVDIVTLRVLCREGVRWRVMAIQDFKPFENLRLMAPISLQVVHVETHRCNISWEISQASHY
FERHLEFEARTLSPGHTWEEAPLLTLKQKQEWICLETLTPDTQYEFQVRVKPLQGEFTTWSPWSQPLA
FRTKPAALGKDT
SEQ ID NO: 193- Anti-PDL1 antibody atezolizumab HC fused with IL-
2Rbeta through a cleavable peptide linker, wherein its Fc contains a
mutation Y407T
EVQLVESGGGLVQPGGSLRLSCAASGFTFSDSWIHWVRQAPGKGLEWVAWISPYGGSTYYADSVKGRF
TISADTSKNTAYLQMNSLRAEDTAVYYCARRHWPGGFDYWGQGTLVTVSSASTKGPSVFPLAPSSKST
SGGTAALGCLVKDYFPEPVTVSWNSGALTSGVHTFPAVLQSSGLYSLSSVVTVPSSSLGTQTYICNVN
HKPSNTKVDKKVEPKSCDKTHTCPPCPAPELLGGPSVFLFPPKPKDTLMISRTPEVTCVVVDVSHEDP
EVKFNWYVDGVEVHNAKTKPREEQYASTYRVVSVLTVLHQDWLNGKEYKCKVSNKALPAPIEKTISKA
KGQPREPQVYTLPPSREEMTKNQVSLTCLVKGFYPSDIAVEWESNGQPENNYKTTPPVLDSDGSFELT
SKLTVDKSRWQQGNVFSCSVMHEALHNHYTQKSLSLSPGKGGGGSGGGGSGGGGSLSGRSDNHGGGGS
AVNGTSQFTCFYNSRANISCVWSQDGALQDTSCQVHAWPDRRRWNQTCELLPVSQASWACNLILGAPD
SQKLTTVDIVTLRVLCREGVRWRVMAIQDEKPFENLRLMAPISLQVVHVETHRCNISWEISQASHYFE
RHLEFEARTLSPGHTWEEAPLLTLKQKQEWICLETLTPDTQYEFQVRVKPLQGEFTTWSPWSQPLAFR
TKPAALGKDT
SEQ ID NO: 194- Fc with LALA and hole mutation (L234A-L235A-Y407T)
DKTHTCPPCPAPEAAGGPSVFLFPPKPKDTLMISRTPEVTCVVVDVSHEDPEVKENWYVDGVEVHNAK
TKPREEQYNSTYRVVSVLTVLHQDWLNGKEYKCKVSNKALPAPIEKTISKAKGQPREPQVYTLPPSRE
EMTKNQVSLTCLVKGFYPSDIAVEWESNGQPENNYKTTPPVLDSDGSFFLTSKLTVDKSRWQQGNVES
CSVMHEALHNHYTQKSLSLSPGK
SEQ ID NO: 195-Fc with LALA and hole mutation (L234A-L235A-Y407T)
with beta subunit
DKTHTCPPCPAPEAAGGPSVFLFPPKPKDTLMISRTPEVTCVVVDVSHEDPEVKENWYVDGVEVHNAK
TKPREEQYNSTYRVVSVLTVLHQDWLNGKEYKCKVSNKALPAPIEKTISKAKGQPREPQVYTLPPSRE
EMTKNQVSLTCLVKGFYPSDIAVEWESNGQPENNYKTTPPVLDSDGSFFLTSKLTVDKSRWQQGNVES
CSVMHEALHNHYTQKSLSLSPGKGGGGSGGGGSGGGGSLSGRSDNHGGGGSAVNGTSQFTCFYNSRAN
ISCVWSQDGALQDTSCQVHAWPDRRRWNQTCELLPVSQASWACNLILGAPDSQKLTTVDIVTLRVLCR
EGVRWRVMAIQDFKPFENLRLMAPISLQVVHVETHRCNISWEISQASHYFERHLEFEARTLSPGHTWE
EAPLLTLKQKQEWICLETLTPDTQYEFQVRVKPLQGEFTTWSPWSQPLAFRTKPAALGKDT
SEQ ID NO: 196- Fc with LALA and hole mutation (L234A-L235A-Y407T)
with beta subunit and gamma subunit
DKTHTCPPCPAPEAAGGPSVFLFPPKPKDTLMISRTPEVTCVVVDVSHEDPEVKENWYVDGVEVHNAK
TKPREEQYNSTYRVVSVLTVLHQDWLNGKEYKCKVSNKALPAPIEKTISKAKGQPREPQVYTLPPSRE
EMTKNQVSLTCLVKGFYPSDIAVEWESNGQPENNYKTTPPVLDSDGSFFLTSKLTVDKSRWQQGNVES
CSVMHEALHNHYTQKSLSLSPGKGGGGSGGGGSGGGGISSGLLSSGGSGGSLSGRSDNHGGGGSAVNG
TSQFTCFYNSRANISCVWSQDGALQDTSCQVHAWPDRRRWNQTCELLPVSQASWACNLILGAPDSQKL
TTVDIVTLRVLCREGVRWRVMAIQDEKPFENLRLMAPISLQVVHVETHRCNISWEISQASHYFERHLE
FEARTLSPGHTWEEAPLLTLKQKQEWICLETLTPDTQYEFQVRVKPLQGEFTTWSPWSQPLAFRTKPA
ALGKDTGGGGSGGGGSGGGGSISSGLLSSGGSGGSLSGRSDNHGGGGSGGGGSLNTTILTPNGNEDTT
ADFFLTTMPTDSLSVSTLPLPEVQCFVENVEYMNCTWNSSSEPQPTNLTLHYWYKNSDNDKVKCSHYL
FSEEITSGCQLQKKEIHLYQTFVVQLQDPREPRRQATQMLKLQNLVIPWAPENLTLHKLSESQLELNW
NNRFLNHCLEHLVQYRTDWDHSWTEQSVDYRHKFSLPSVDGQKRYTFRVRSRENPLCGSAQHWSEWSH
PIHWGSNTSKENPFLFALEA
SEQ ID NO: 197- Fc with LALA and hole mutation (L234A-L235A-Y407T)
with beta subunit-2 cleavable substrates
DKTHTCPPCPAPEAAGGPSVFLFPPKPKDTLMISRTPEVTCVVVDVSHEDPEVKENWYVDGVEVHNAK
TKPREEQYNSTYRVVSVLTVLHQDWLNGKEYKCKVSNKALPAPIEKTISKAKGQPREPQVYTLPPSRE
EMTKNQVSLTCLVKGFYPSDIAVEWESNGQPENNYKTTPPVLDSDGSFELTSKLTVDKSRWQQGNVES
CSVMHEALHNHYTQKSLSLSPGKGGGGSGGGGSGGGGISSGLLSSGGSGGSLSGRSDNHGGGGSAVNG
TSQFTCFYNSRANISCVWSQDGALQDTSCQVHAWPDRRRWNQTCELLPVSQASWACNLILGAPDSQKL
TTVDIVTLRVLCREGVRWRVMAIQDFKPFENLRLMAPISLQVVHVETHRCNISWEISQASHYFERHLE
FEARTLSPGHTWEEAPLLTLKQKQEWICLETLTPDTQYEFQVRVKPLQGEFTTWSPWSQPLAFRTKPA
ALGKDT
SEQ ID NO: 198- Fc with LALA and hole mutation (L234A-L235A-Y407T)
with a dimer of the IL-2R beta subunit
DKTHTCPPCPAPEAAGGPSVFLFPPKPKDTLMISRTPEVTCVVVDVSHEDPEVKENWYVDGVEVHNAK
TKPREEQYNSTYRVVSVLTVLHQDWLNGKEYKCKVSNKALPAPIEKTISKAKGQPREPQVYTLPPSRE
EMTKNQVSLTCLVKGFYPSDIAVEWESNGQPENNYKTTPPVLDSDGSFELTSKLTVDKSRWQQGNVES
CSVMHEALHNHYTQKSLSLSPGKGGGGSGGGGSGGGGISSGLLSSGGSGGSLSGRSDNHGGGGSAVNG
TSQFTCFYNSRANISCVWSQDGALQDTSCQVHAWPDRRRWNQTCELLPVSQASWACNLILGAPDSQKL
TTVDIVTLRVLCREGVRWRVMAIQDFKPFENLRLMAPISLQVVHVETHRCNISWEISQASHYFERHLE
FEARTLSPGHTWEEAPLLTLKQKQEWICLETLTPDTQYEFQVRVKPLQGEFTTWSPWSQPLAFRTKPA
ALGKDTGGGGSGGGGSGGGGSISSGLLSSGGSGGSLSGRSDNHGGGGSGGGGSAVNGTSQFTCFYNSR
ANISCVWSQDGALQDTSCQVHAWPDRRRWNQTCELLPVSQASWACNLILGAPDSQKLTTVDIVTLRVL
CREGVRWRVMAIQDFKPFENLRLMAPISLQVVHVETHRCNISWEISQASHYFERHLEFEARTLSPGHT
WEEAPLLTLKQKQEWICLETLTPDTQYEFQVRVKPLQGEFTTWSPWSQPLAFRTKPAALGKDT
SEQ ID NO: 199- Fc with LALA and hole mutation (L234A-L235A-Y407T)
fused with beta subunit containing mutation R15Y
DKTHTCPPCPAPEAAGGPSVELFPPKPKDTLMISRTPEVTCVVVDVSHEDPEVKENWYVDGVEVHNAK
TKPREEQYNSTYRVVSVLTVLHQDWLNGKEYKCKVSNKALPAPIEKTISKAKGQPREPQVYTLPPSRE
EMTKNQVSLTCLVKGFYPSDIAVEWESNGQPENNYKTTPPVLDSDGSFELTSKLTVDKSRWQQGNVES
CSVMHEALHNHYTQKSLSLSPGKGGGGSGGGGSGGGGISSGLLSSGGSGGSLSGRSDNHGGGGSAVNG

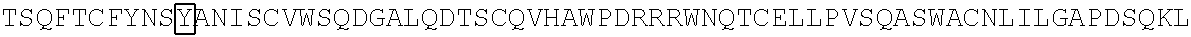

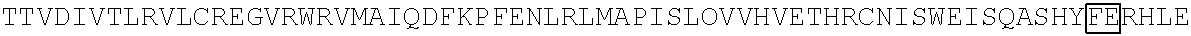

FEARTLSPGHTWEEAPLLTLKQKQEWICLETLTPDTQYEFQVRVKPLQGEFTTWSPWSQPLAFRTKPA
ALGKDT
SEQ ID NO: 200- Fc with LALA and hole mutation (L234A-L235A-Y407T)
fused with beta subunit containing mutation (D68E)
DKTHTCPPCPAPEAAGGPSVFLFPPKPKDTLMISRTPEVTCVVVDVSHEDPEVKFNWYVDGVEVHNAK
TKPREEQYNSTYRVVSVLTVLHQDWLNGKEYKCKVSNKALPAPIEKTISKAKGQPREPQVYTLPPSRE
EMTKNQVSLTCLVKGFYPSDIAVEWESNGQPENNYKTTPPVLDSDGSFELTSKLTVDKSRWQQGNVES
CSVMHEALHNHYTQKSLSLSPGKGGGGSGGGGSGGGGISSGLLSSGGSGGSLSGRSDNHGGGGSAVNG

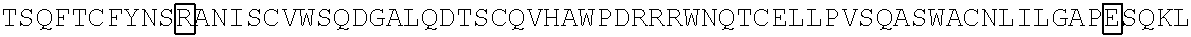

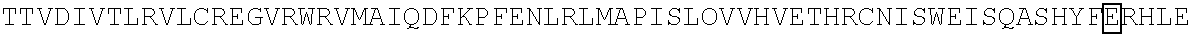

FEARTLSPGHTWEEAPLLTLKQKQEWICLETLTPDTQYEFQVRVKPLQGEFTTWSPWSQPLAFRTKPA
ALGKDT
SEQ ID NO: 201- Fc with LALA and hole mutation (L234A-L235A-Y407T)
fused with beta subunit containing mutation (S69H)
DKTHTCPPCPAPEAAGGPSVFLFPPKPKDTLMISRTPEVTCVVVDVSHEDPEVKENWYVDGVEVHNAK
TKPREEQYNSTYRVVSVLTVLHQDWLNGKEYKCKVSNKALPAPIEKTISKAKGQPREPQVYTLPPSRE
EMTKNQVSLTCLVKGFYPSDIAVEWESNGQPENNYKTTPPVLDSDGSFFLTSKLTVDKSRWQQGNVES
CSVMHEALHNHYTQKSLSLSPGKGGGGSGGGGSGGGGISSGLLSSGGSGGSLSGRSDNHGGGGSAVNG

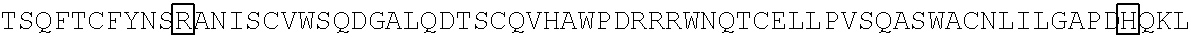

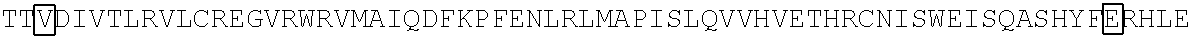

FEARTLSPGHTWEEAPLLTLKQKQEWICLETLTPDTQYEFQVRVKPLQGEFTTWSPWSQPLAFRTKPA
ALGKDT
SEQ ID NO: 202- Fc with LALA and hole mutation (L234A-L235A-Y407T)
fused with beta subunit containing mutation (V75Q)
DKTHTCPPCPAPEAAGGPSVFLFPPKPKDTLMISRTPEVTCVVVDVSHEDPEVKENWYVDGVEVHNAK
TKPREEQYNSTYRVVSVLTVLHQDWLNGKEYKCKVSNKALPAPIEKTISKAKGQPREPQVYTLPPSRE
EMTKNQVSLTCLVKGFYPSDIAVEWESNGQPENNYKTTPPVLDSDGSFELTSKLTVDKSRWQQGNVES
CSVMHEALHNHYTQKSLSLSPGKGGGGSGGGGSGGGGISSGLLSSGGSGGSLSGRSDNHGGGGSAVNG

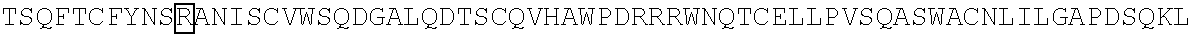

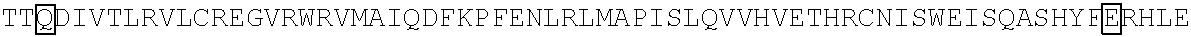

FEARTLSPGHTWEEAPLLTLKQKQEWICLETLTPDTQYEFQVRVKPLQGEFTTWSPWSQPLAFRTKPA
ALGKDT
SEQ ID NO: 203- Fc with LALA and hole mutation (L234A-L235A-Y407T)
fused with beta subunit containing mutation (V75F)
DKTHTCPPCPAPEAAGGPSVFLFPPKPKDTLMISRTPEVTCVVVDVSHEDPEVKENWYVDGVEVHNAK
TKPREEQYNSTYRVVSVLTVLHQDWLNGKEYKCKVSNKALPAPIEKTISKAKGQPREPQVYTLPPSRE
EMTKNQVSLTCLVKGFYPSDIAVEWESNGQPENNYKTTPPVLDSDGSFELTSKLTVDKSRWQQGNVES
CSVMHEALHNHYTQKSLSLSPGKGGGGSGGGGSGGGGISSGLLSSGGSGGSLSGRSDNHGGGGSAVNG

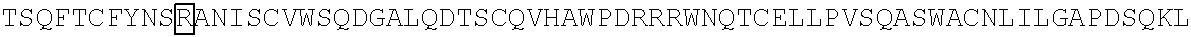

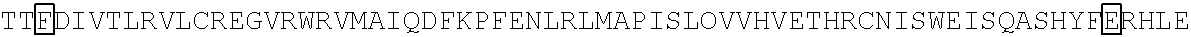

FEARTLSPGHTWEEAPLLTLKQKQEWICLETLTPDTQYEFQVRVKPLQGEFTTWSPWSQPLAFRTKPA
ALGKDT
SEQ ID NO: 204- Fc with LALA and hole mutation (L234A-L235A-Y407T)
fused with beta subunit containing mutation (E136Q)
DKTHTCPPCPAPEAAGGPSVFLFPPKPKDTLMISRTPEVTCVVVDVSHEDPEVKENWYVDGVEVHNAK
TKPREEQYNSTYRVVSVLTVLHQDWLNGKEYKCKVSNKALPAPIEKTISKAKGQPREPQVYTLPPSRE
EMTKNQVSLTCLVKGFYPSDIAVEWESNGQPENNYKTTPPVLDSDGSFELTSKLTVDKSRWQQGNVES
CSVMHEALHNHYTQKSLSLSPGKGGGGSGGGGSGGGGISSGLLSSGGSGGSLSGRSDNHGGGGSAVNG

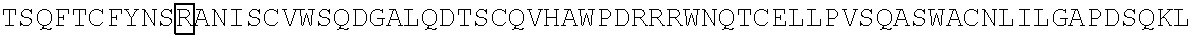

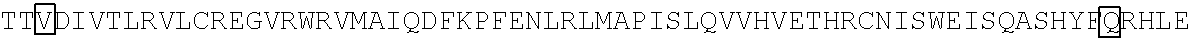

FEARTLSPGHTWEEAPLLTLKQKQEWICLETLTPDTQYEFQVRVKPLQGEFTTWSPWSQPLAFRTKPA
ALGKDT
SEQ ID NO: 205- Anti-PDL1 atezolizumab HC with LALA and hole
mutation (L234A-L235A-Y407T) fused with beta subunit containing
mutation (E136Q/H138R)
DKTHTCPPCPAPEAAGGPSVFLFPPKPKDTLMISRTPEVTCVVVDVSHEDPEVKENWYVDGVEVHNAK
TKPREEQYNSTYRVVSVLTVLHQDWLNGKEYKCKVSNKALPAPIEKTISKAKGQPREPQVYTLPPSRE
EMTKNQVSLTCLVKGFYPSDIAVEWESNGQPENNYKTTPPVLDSDGSFELTSKLTVDKSRWQQGNVES
CSVMHEALHNHYTQKSLSLSPGKGGGGSGGGGSGGGGISSGLLSSGGSGGSLSGRSDNHGGGGSAVNG

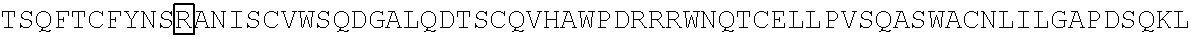

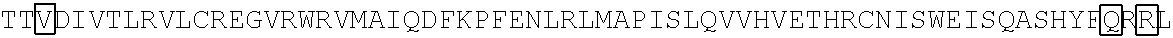

EFEARTLSPGHTWEEAPLLTLKQKQEWICLETLTPDTQYEFQVRVKPLQGEFTTWSPWSQPLAFRTKP
AALGKDT
SEQ ID NO: 206- Anti-PDL1 atezolizumab HC with a hole mutation
(Y407T)- with beta, single cleavable site (underlined)
MGWTLVFLFLLSVTAGVHSEVQLVESGGGLVQPGGSLRLSCAASGFTFSDSWIHWVRQAPGKGLEWVA
WISPYGGSTYYADSVKGRFTISADTSKNTAYLQMNSLRAEDTAVYYCARRHWPGGFDYWGQGTLVTVS
SASTKGPSVFPLAPSSKSTSGGTAALGCLVKDYFPEPVTVSWNSGALTSGVHTFPAVLQSSGLYSLSS
VVTVPSSSLGTQTYICNVNHKPSNTKVDKKVEPKSCDKTHTCPPCPAPELLGGPSVFLFPPKPKDTLM
ISRTPEVTCVVVDVSHEDPEVKENWYVDGVEVHNAKTKPREEQYASTYRVVSVLTVLHQDWLNGKEYK
CKVSNKALPAPIEKTISKAKGQPREPQVYTLPPSREEMTKNQVSLTCLVKGFYPSDIAVEWESNGQPE
NNYKTTPPVLDSDGSFFLTSKLTVDKSRWQQGNVFSCSVMHEALHNHYTQKSLSLSPGKGGSLSGRSD
NHGGGGSAVNGTSQFTCFYNSRANISCVWSQDGALQDTSCQVHAWPDRRRWNQTCELLPVSQASWACN
LILGAPDSQKLTTVDIVTLRVLCREGVRWRVMAIQDEKPFENLRLMAPISLQVVHVETHRCNISWEIS
QASHYFERHLEFEARTLSPGHTWEEAPLLTLKQKQEWICLETLTPDTQYEFQVRVKPLQGEFTTWSPW
SQPLAFRTKPAALGKDT
SEQ ID NO: 207- Anti-PDL1 atezolizumab HC with a hole mutation
(Y407T)- with beta, single cleavable site (underlined)
MGWTLVFLFLLSVTAGVHSEVQLVESGGGLVQPGGSLRLSCAASGFTFSDSWIHWVRQAPGKGLEWVA
WISPYGGSTYYADSVKGRFTISADTSKNTAYLQMNSLRAEDTAVYYCARRHWPGGFDYWGQGTLVTVS
SASTKGPSVFPLAPSSKSTSGGTAALGCLVKDYFPEPVTVSWNSGALTSGVHTFPAVLQSSGLYSLSS
VVTVPSSSLGTQTYICNVNHKPSNTKVDKKVEPKSCDKTHTCPPCPAPELLGGPSVFLFPPKPKDTLM
ISRTPEVTCVVVDVSHEDPEVKENWYVDGVEVHNAKTKPREEQYASTYRVVSVLTVLHQDWLNGKEYK
CKVSNKALPAPIEKTISKAKGQPREPQVYTLPPSREEMTKNQVSLTCLVKGFYPSDIAVEWESNGQPE
NNYKTTPPVLDSDGSFFLTSKLTVDKSRWQQGNVFSCSVMHEALHNHYTQKSLSLSPGKGGSLSGRSD
NHGSAVNGTSQFTCFYNSRANISCVWSQDGALQDTSCQVHAWPDRRRWNQTCELLPVSQASWACNLIL
GAPDSQKLTTVDIVTLRVLCREGVRWRVMAIQDFKPFENLRLMAPISLQVVHVETHRCNISWEISQAS
HYFERHLEFEARTLSPGHTWEEAPLLTLKQKQEWICLETLTPDTQYEFQVRVKPLQGEFTTWSPWSQP
LAFRTKPAALGKDT
SEQ ID NO: 208- Anti-PDL1 atezolizumab HC with a hole mutation
(Y407T)- with beta D68E (boxed), single cleavable site
(underlined)
MGWTLVFLFLLSVTAGVHSEVQLVESGGGLVQPGGSLRLSCAASGFTFSDSWIHWVRQAPGKGLEWVA
WISPYGGSTYYADSVKGRFTISADTSKNTAYLQMNSLRAEDTAVYYCARRHWPGGFDYWGQGTLVTVS
SASTKGPSVFPLAPSSKSTSGGTAALGCLVKDYFPEPVTVSWNSGALTSGVHTFPAVLQSSGLYSLSS
VVTVPSSSLGTQTYICNVNHKPSNTKVDKKVEPKSCDKTHTCPPCPAPELLGGPSVFLFPPKPKDTLM
ISRTPEVTCVVVDVSHEDPEVKENWYVDGVEVHNAKTKPREEQYASTYRVVSVLTVLHQDWLNGKEYK
CKVSNKALPAPIEKTISKAKGQPREPQVYTLPPSREEMTKNQVSLTCLVKGFYPSDIAVEWESNGQPE
NNYKTTPPVLDSDGSFFLTSKLTVDKSRWQQGNVFSCSVMHEALHNHYTQKSLSLSPGKGGSLSGRSD
NHGSAVNGTSQFTCFYNSRANISCVWSQDGALQDTSCQVHAWPDRRRWNQTCELLPVSQASWACNLIL

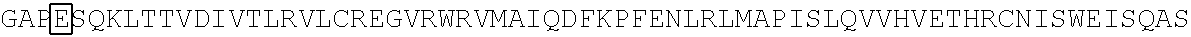

HYFERHLEFEARTLSPGHTWEEAPLLTLKQKQEWICLETLTPDTQYEFQVRVKPLQGEFTTWSPWSQP
LAFRTKPAALGKDT
SEQ ID NO: 209- 589A-Humanized heavy chain (HC)- Knob mutations
(boxed)- IL2 T-3A/C125S-R38S/F42A/Y45A/E62A (underlined), linker
(italicized)
EVQLVESGGGLVKPGGSLRLSCAVSGFY FNRGYWICWVRQAPGKGLEWIGCIDTGSGVPYYANWAKGR
FTISRHTSKTTLTLQMNSLRAEDTASYFCARNSDSIYENLWGPGTLVTVSSASTKGPSVFPLAPSSKS
TSGGTAALGCLVKDY FPEPVTVSWNSGALTSGVHT FPAVLQSSGLYSLSSVVTVPSSSLGTQTYICNV
NHKPSNTKVDKKVEPKSCDKTHTCPPCPAPELLGGPSVFLFPPKPKDTLMISRTPEVTCVVVDVSHED
PEVKFNWYVDGVEVHNAKTKPREEQYNSTYRVVSVLTVLHQDWLNGKEYKCKVSNKALPAPIEKTISK

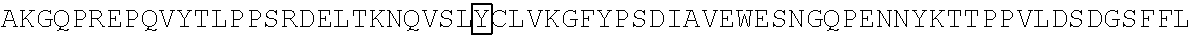

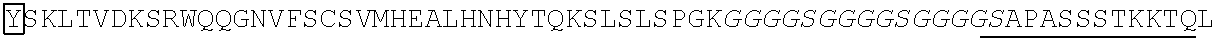

QLEHLLLDLQMILNGINNYKNPKLTSMLTAKFAMPKKATELKHLQCLEEALKPLEEVLNLAQSKNEHL
RPRDLISNINVIVLELKGSETTEMCEYADETATIVEFLNRWITESQSIISTLT*
SEQ ID NO: 210- 589A-Humanized HC- Hole mutations (boxed)- with
Beta subunit of extracellular domain, also referred to as “beta”
(underlined), cleavable (cleavable linker is italicized)
EVQLVESGGGLVKPGGSLRLSCAVSGFYFNRGYWICWVRQAPGKGLEWIGCIDTGSGVPYYANWAKGR
FTISRHTSKTTLTLQMNSLRAEDTASYFCARNSDSIYFNLWGPGTLVTVSSASTKGPSVFPLAPSSKS
TSGGTAALGCLVKDYFPEPVTVSWNSGALTSGVHTFPAVLQSSGLYSLSSVVTVPSSSLGTQTYICNV
NHKPSNTKVDKKVEPKSCDKTHTCPPCPAPELLGGPSVELFPPKPKDTLMISRTPEVTCVVVDVSHED
PEVKFNWYVDGVEVHNAKTKPREEQYNSTYRVVSVLTVLHQDWLNGKEYKCKVSNKALPAPIEKTISK

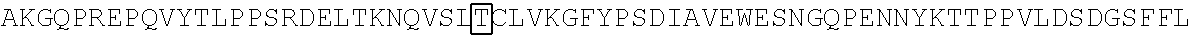

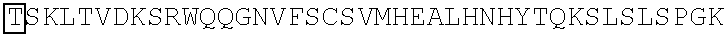

GGGGSGGGGSGGGGSISSGLLSSGGSGGSLSGRSDNHGGGGSGGGGSAVNGTSQFTCFYNSRANISCV
WSQDGALQDTSCQVHAWPDRRRWNQTCELLPVSQASWACNLILGAPDSQKLTTVDIVTLRVLCREGVR
WRVMAIQDFKPFENLRLMAPISLQVVHVETHRCNISWEISQASHYFERHLEFEARTLSPGHTWEEAPL
LTLKQKQEWICLETLTPDTQYEFQVRVKPLQGEFTTWSPWSQPLAFRTKPAALGKDT*
SEQ ID NO: 211- 589A-Humanized HC- Hole mutations (boxed)- with
Beta (underlined), not-cleavable
EVQLVESGGGLVKPGGSLRLSCAVSGFYFNRGYWICWVRQAPGKGLEWIGCIDTGSGVPYYANWAKGR
FTISRHTSKTTLTLQMNSLRAEDTASYFCARNSDSIYFNLWGPGTLVTVSSASTKGPSVFPLAPSSKS
TSGGTAALGCLVKDYFPEPVTVSWNSGALTSGVHTFPAVLQSSGLYSLSSVVTVPSSSLGTQTYICNV
NHKPSNTKVDKKVEPKSCDKTHTCPPCPAPELLGGPSVFLFPPKPKDTLMISRTPEVTCVVVDVSHED
PEVKFNWYVDGVEVHNAKTKPREEQYNSTYRVVSVLTVLHQDWLNGKEYKCKVSNKALPAPIEKTISK

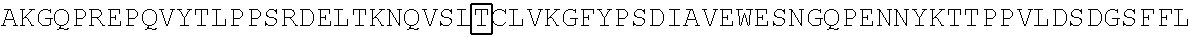

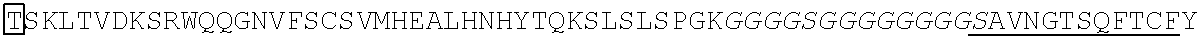

NSRANISCVWSQDGALQDTSCQVHAWPDRRRWNQTCELLPVSQASWACNLILGAPDSQKLTTVDIVTL
RVLCREGVRWRVMAIQDFKPFENLRLMAPISLQVVHVETHRCNISWEISQASHYFERHLEFEARTLSP
GHTWEEAPLLTLKQKQEWICLETLTPDTQYEFQVRVKPLQGEFTTWSPWSQPLAFRTKPAALGKDT*
SEQ ID NO: 212- 589A- Humanized HC Hole mutations (boxed)- with
Beta (underlined), single cleavable site (underlined and italicized)
EVQLVESGGGLVKPGGSLRLSCAVSGFYFNRGYWICWVRQAPGKGLEWIGCIDTGSGVPYYANWAKGR
FTISRHTSKTTLTLQMNSLRAEDTASYFCARNSDSIYFNLWGPGTLVTVSSASTKGPSVFPLAPSSKS
TSGGTAALGCLVKDYFPEPVTVSWNSGALTSGVHTFPAVLQSSGLYSLSSVVTVPSSSLGTQTYICNV
NHKPSNTKVDKKVEPKSCDKTHTCPPCPAPELLGGPSVELFPPKPKDTLMISRTPEVTCVVVDVSHED
PEVKFNWYVDGVEVHNAKTKPREEQYNSTYRVVSVLTVLHQDWLNGKEYKCKVSNKALPAPIEKTISK

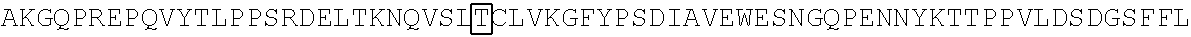

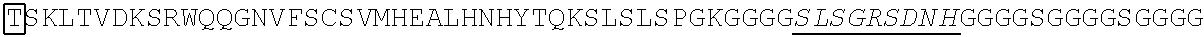

SAVNGTSQFTCFYNSRANISCVWSQDGALQDTSCQVHAWPDRRRWNQTCELLPVSQASWACNLILGAP
DSQKLTTVDIVTLRVLCREGVRWRVMAIQDFKPFENLRLMAPISLQVVHVETHRCNISWEISQASHYF
ERHLEFEARTLSPGHTWEEAPLLTLKQKQEWICLETLTPDTQYEFQVRVKPLQGEFTTWSPWSQPLAF
RTKPAALGKDT*
SEQ ID NO: 213- 589A-Humanized HC- Hole mutations (boxed- with
Beta (underlined), single cleavable site 2 (underlined and
italicized)
EVQLVESGGGLVKPGGSLRLSCAVSGFYFNRGYWICWVRQAPGKGLEWIGCIDTGSGVPYYANWAKGR
FTISRHTSKTTLTLQMNSLRAEDTASYFCARNSDSIYFNLWGPGTLVTVSSASTKGPSVFPLAPSSKS
TSGGTAALGCLVKDYFPEPVTVSWNSGALTSGVHTFPAVLQSSGLYSLSSVVTVPSSSLGTQTYICNV
NHKPSNTKVDKKVEPKSCDKTHTCPPCPAPELLGGPSVFLFPPKPKDTLMISRTPEVTCVVVDVSHED
PEVKFNWYVDGVEVHNAKTKPREEQYNSTYRVVSVLTVLHQDWLNGKEYKCKVSNKALPAPIEKTISK

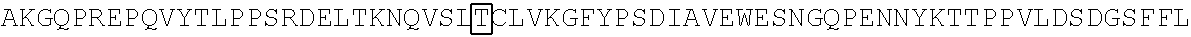

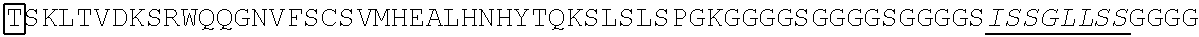

SAVNGTSQFTCFYNSRANISCVWSQDGALQDTSCQVHAWPDRRRWNQTCELLPVSQASWACNLILGAP
DSQKLTTVDIVTLRVLCREGVRWRVMAIQDEKPFENLRLMAPISLQVVHVETHRCNISWEISQASHYF
ERHLEFEARTLSPGHTWEEAPLLTLKQKQEWICLETLTPDTQYEFQVRVKPLQGEFTTWSPWSQPLAF
RTKPAALGKDT*
SEQ ID NO: 214- 589A-Humanized HC- Hole- with Beta (underlined),
single cleavable site (underlined and italicized)/shorter linker
(italicized)
DIQMTQSPSSVSASVGDRVTITCQASQSIGGYLSWYQQKPGQPPKLLIYKASTLASGVPSRFKGSGSG
TDFTLTISSLDSEDAATYYCQNYAGVSIYGAVFGGGTKVVVKRTVAAPSVFIFPPSDEQLKSGTASVV
CLLNNFYPREAKVQWKVDNALQSGNSQESVTEQDSKDSTYSLSSTLTLSKADYEKHKVYACEVTHQGL
SSPVTKSFNRGECEVQLVESGGGLVKPGGSLRLSCAVSGFYENRGYWICWVRQAPGKGLEWIGCIDTG
SGVPYYANWAKGRFTISRHTSKTTLTLQMNSLRAEDTASYFCARNSDSIYFNLWGPGTLVTVSSASTK
GPSVFPLAPSSKSTSGGTAALGCLVKDYFPEPVTVSWNSGALTSGVHTFPAVLQSSGLYSLSSVVTVP
SSSLGTQTYICNVNHKPSNTKVDKKVEPKSCDKTHTCPPCPAPELLGGPSVFLFPPKPKDTLMISRTP
EVTCVVVDVSHEDPEVKENWYVDGVEVHNAKTKPREEQYNSTYRVVSVLTVLHQDWLNGKEYKCKVSN

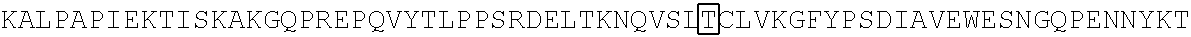

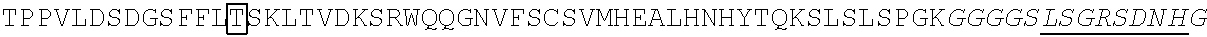

GGGSAVNGTSQFTCFYNSRANISCVWSQDGALQDTSCQVHAWPDRRRWNQTCELLPVSQASWACNLIL
GAPDSQKLTTVDIVTLRVLCREGVRWRVMAIQDEKPFENLRLMAPISLQVVHVETHRCNISWEISQAS
HYFERHLEFEARTLSPGHTWEEAPLLTLKQKQEWICLETLTPDTQYEFQVRVKPLQGEFTTWSPWSQP
LAFRTKPAALGKDT**
SEQ ID NO: 215- 589A-Humanized HC- Hole mutations (boxed)- with
Beta (underlined), single cleavable site/shorter linker
(underlined and italicized)/beta mutation D68E (boxed, underlined
and bolded)
EVQLVESGGGLVKPGGSLRLSCAVSGFYFNRGYWICWVRQAPGKGLEWIGCIDTGSGVPYYANWAKGR
FTISRHTSKTTLTLQMNSLRAEDTASYFCARNSDSIYFNLWGPGTLVTVSSASTKGPSVFPLAPSSKS
TSGGTAALGCLVKDYFPEPVTVSWNSGALTSGVHTFPAVLQSSGLYSLSSVVTVPSSSLGTQTYICNV
NHKPSNTKVDKKVEPKSCDKTHTCPPCPAPELLGGPSVELFPPKPKDTLMISRTPEVTCVVVDVSHED
PEVKFNWYVDGVEVHNAKTKPREEQYNSTYRVVSVLTVLHQDWLNGKEYKCKVSNKALPAPIEKTISK

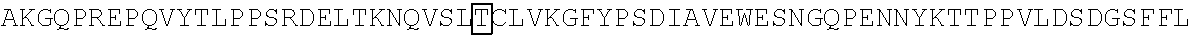

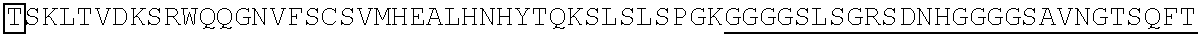

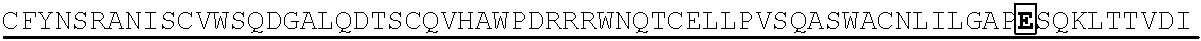

VTLRVLCREGVRWRVMAIQDFKPFENLRLMAPISLQVVHVETHRCNISWEISQASHYFERHLEFEART
LSPGHTWEEAPLLTLKQKQEWICLETLTPDTQYEFQVRVKPLQGEFTTWSPWSQPLAFRTKPAALGKD
T
SEQ ID NO: 216- 589A-Humanized LC- 589A LC
DIQMTQSPSSVSASVGDRVTITCQASQSIGGYISWYQQKPGQPPKLLIYKASTLASGVPSRFKGSGSG
TDFTLTISSLDSEDAATYYCQNYAGVSIYGAVFGGGTKVVVKRTVAAPSVFIFPPSDEQLKSGTASVV
CLLNNFYPREAKVQWKVDNALQSGNSQESVTEQDSKDSTYSLSSTLTLSKADYEKHKVYACEVTHQGL
SSPVTKSENRGEC
SEQ ID NO: 217- cleavable peptide linker
GGGGSGGGGSGGGGSISSGLLSSGGSGGSLSGRSDNHGGGGSGGGGS

1-50. (canceled)

51. A prodrug comprising a cytokine moiety, a masking moiety, and a carrier moiety, wherein
the masking moiety binds to the cytokine moiety and inhibits a biological activity of the cytokine moiety,
the cytokine moiety is a human IL-2 agonist polypeptide comprising an amino acid sequence selected from SEQ ID NOs: 8-17, 19-33, 36, 37, and 39-46 and is fused to the carrier moiety,
the masking moiety is fused to the carrier moiety through a cleavable peptide linker and comprises an extracellular domain (ECD) of a human IL-2Rβ polypeptide, and
the carrier moiety is an antibody Fc domain or an antibody, wherein the carrier moiety comprises knobs-into-holes mutations, and wherein the cytokine moiety and the masking moiety are fused to different polypeptide chains of the antibody Fc domain or to the different heavy chains of the antibody.

52. The prodrug of claim 51, wherein the masking moiety comprises two copies of the ECD of human IL-2Rβ fused together through a peptide linker.

53. The prodrug of claim 51, wherein the ECD of the human IL-2Rβ polypeptide comprises SEQ ID NO: 3, 4, or 5.

54. The prodrug of claim 51, wherein the cytokine moiety is fused to the carrier moiety through a noncleavable peptide linker.

55. The prodrug of claim 54, wherein the noncleavable peptide linker is selected from SEQ ID NOs: 47-51.

56. The prodrug of claim 51, wherein the cleavable peptide linker comprises a substrate sequence of urokinase-type plasminogen activator (uPA), matrix metallopeptidase (MMP) 2, or MMP9.

57. The prodrug of claim 56, wherein the cleavable peptide linker comprises substrate sequences of (i) both uPA and MMP2, (ii) both uPA and MMP9, or (iii) uPA, MMP2 and MMP9.

58. The prodrug of claim 56, wherein the cleavable peptide linker comprises an amino acid sequence selected from SEQ ID NOs: 18, 34, 35, 38, 52-121, and 217.

59. The prodrug of claim 51, wherein the cleavable peptide linker is cleavable by one or more proteases located at a tumor site or its surrounding environment, and the cleavage leads to activation of the prodrug at the tumor site or surrounding environment.

60. The prodrug of claim 51, wherein the carrier moiety comprises mutations L234A and L235A (“LALA”) (EU numbering).

61. The prodrug of claim 51, wherein the cytokine moiety and the masking moiety are fused to the C-termini of the two different polypeptide chains of the Fc domain or to the C-termini of the two different heavy chains of the antibody.

62. The prodrug of claim 51, wherein the cytokine moiety and the masking moiety are fused to the N-termini of the two different polypeptide chains of the Fc domain or to the N-termini of the two different heavy chains of the antibody.

63. The prodrug of claim 51, wherein the knobs-into-holes mutations comprise a T366Y “knob” mutation on a polypeptide chain of the Fc domain or a heavy chain of the antibody, and a Y407T “hole” mutation in the other polypeptide of the Fc domain or the other heavy chain of the antibody (EU numbering).

64. The prodrug of claim 51, wherein the knobs-into-holes mutations comprise Y349C and/or T366W mutations in the CH3 domain of the “knob chain” and E356C, T366S, L368A, and/or Y407V mutations in the CH3 domain of the “hole chain” (EU numbering).

65. A prodrug comprising a cytokine moiety, a masking moiety, and a carrier moiety, wherein the carrier moiety is an antibody Fc domain comprising two polypeptide chains whose amino acid sequences respectively comprise an amino acid sequence selected from SEQ ID NOs: 195, 197, 198, and 200 and an amino acid sequence selected from SEQ ID NOs: 132-137 and 139.

66. The prodrug of claim 51, wherein the carrier moiety is an antibody or an antibody Fc domain that specifically binds to one or more antigens selected from Guanyl cyclase C (GCC), carbohydrate antigen 19-9 (CA19-9), glycoprotein A33 (gpA33), mucin 1 (MUC1), carcinoembryonic antigen (CEA), insulin-like growth factor 1 receptor (IGF1-R), human epidermal growth factor receptor 2 (HER2), human epidermal growth factor receptor 3 (HER3), delta-like protein 3 (DLL3), delta-like protein 4 (DLL4), epidermal growth factor receptor (EGFR), glypican-3 (GPC3), c-MET, vascular endothelial growth factor receptor 1 (VEGFR1), vascular endothelial growth factor receptor 2 (VEGFR2), Nectin-4, Liv-1, glycoprotein NMB (GPNMB), prostate specific membrane antigen (PSMA), Trop-2, carbonic anhydrase IX (CA9), endothelin B receptor (ETBR), six transmembrane epithelial antigen of the prostate 1 (STEAP1), folate receptor alpha (FR-α), SLIT and NTRK-like protein 6 (SLITRK6), carbonic anhydrase VI (CA6), ectonucleotide pyrophosphatase/phosphodiesterase family member 3 (ENPP3), mesothelin, trophoblast glycoprotein (TPBG), CD19, CD20, CD22, CD33, CD40, CD56, CD66e, CD70, CD74, CD79b, CD98, CD123, CD138, CD352, CD47, signal-regulatory protein alpha (SIRPα), 5T4, BCMA, PD-L1, PD-1, Fibroblast Activation Protein alpha (FAPalpha), the Melanoma-associated Chondroitin Sulfate Proteoglycan (MCSP), and EPCAM.

67. The prodrug of claim 51, wherein the carrier moiety is an antibody comprising two heavy chains whose amino acid sequences respectively comprise SEQ ID NO: 191 and one of SEQ ID NOs: 192, 193, and 206-208, and two light chains whose amino acid sequence comprises SEQ ID NO: 189.

68. A pharmaceutical composition comprising the prodrug of claim 51 and a pharmaceutically acceptable excipient.

69. A polynucleotide or polynucleotides encoding the prodrug of claim 51.

70. An expression vector or vectors comprising the polynucleotide or polynucleotides of claim 69.

71. A host cell comprising the vector(s) of claim 70.

72. A method of making the prodrug of claim 51, comprising
culturing a host cell comprising expression vector(s) encoding the prodrug of claim 51 under conditions that allow expression of the prodrug, wherein the host cell is a mammalian cell, and
isolating the prodrug.

73. The prodrug of claim 65, comprising:
SEQ ID NOs: 195 and 132,
SEQ ID NOs: 197 and 132,
SEQ ID NOs: 198 and 132,
SEQ ID NOs: 198 and 133, or
SEQ ID NOs: 198 and 136.

74. A pharmaceutical composition comprising the prodrug of claim 65 and a pharmaceutically acceptable excipient.

75. A polynucleotide or polynucleotides encoding the prodrug of claim 65.

76. An expression vector or vectors comprising the polynucleotide or polynucleotides of claim 75.

77. A host cell comprising the vector(s) of claim 76.

78. A method of making the prodrug of claim 65, comprising
culturing a host cell comprising expression vector(s) encoding the prodrug of claim 65 under conditions that allow expression of the prodrug, wherein the host cell is a mammalian cell, and
isolating the prodrug.


SEQUENCE LISTING (217 sequences)

SEQ ID NO: 1 - protein, 133 aa, Homo sapiens [sequence omitted]

SEQ ID NO: 2 - protein, 133 aa, Homo sapiens
GIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 3 - protein, 214 aa, Homo sapiens
AVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQGEFTTWSPWSQPLAFRTKPAALGKDT

SEQ ID NO: 4 - protein, 214 aa, synthetic construct
  REGION 1..214: note = source = /note="Description of Artificial Sequence: Synthetic polypeptide"
AVNGTSQFTCFYNSRANISCVWSQDGALQDTSCQVHAWPDRRRWNQTCELLPVSQASWACNLILGAPESQKLTTVDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQGEFTTWSPWSQPLAFRTKPAALGKDT

SEQ ID NO: 5 - protein, 214 aa, synthetic construct
  REGION 1..214: note = source = /note="Description of Artificial Sequence: Synthetic polypeptide"
AVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFQRRLEFEARTLSPGHTWEEAPLLTLKQKQEWICLETLTPDTQYEFQVRVKPLQGEFTTWSPWSQPLAFRTKPAALGKDT

SEQ ID NO: 6 - protein, 240 aa, Homo sapiens
LNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLQNLVIPWAPENLTLHKLSESQLELNWNNRFLNHCLEHLVQYRTDWDHSWTEQSVDYRHKFSLPSVDGQKRYTFRVRSRFNPLCGSAQHWSEWSHPIHWGSNTSKENPFLFALEA

SEQ ID NO: 7 - protein, 77 aa, Homo sapiens
ITCPPPMSVEHADIWVKSYSLYSRERYICNSGFKRKAGTSSLTECVLNKATNVAHWTTPSLKCIRDPALVHQRPAPP

SEQ ID NO: 8 - protein, 133 aa, synthetic construct
  VARIANT 3: note = /replace="Ala"
  VARIANT 35: note = /replace="Asn"
  VARIANT 42: note = /replace="Gly" or "Ser" or "Thr" or "Gln" or "Glu" or "Asn" or "Asp" or "Arg" or "Lys"
  VARIANT 45: note = /replace="Gly" or "Ser" or "Thr" or "Gln" or "Glu" or "Asn" or "Asp" or "Arg" or "Lys"
  VARIANT 72: note = /replace="Gly" or "Ser" or "Thr" or "Gln" or "Glu" or "Asn" or "Asp" or "Arg" or "Lys"
  VARIANT 73: note = /replace="Thr"
  VARIANT 125: note = /replace="Ser"
  SITE 1..133: note = /note="Variant residues given in the sequence have no preference with respect to those in the annotations for variant positions"
  REGION 1..133: note = source = /note="Description of Artificial Sequence: Synthetic polypeptide"
APNSSSTKKTQLQLEHLLLDLQMILNGINNYKNPKLTRMLTAKFAMPKKATELKHLQCLEEELKPLEEVLNAAQSKNFHLRPRDLISNINVIVLELKGSETTFMCEYADETATIVEFLNRWITFCQSIISTLT

SEQ ID NO: 9 - protein, 133 aa, synthetic construct
  REGION 1..133: note = source = /note="Description of Artificial Sequence: Synthetic polypeptide"
APASSSTKKTQLQLEHLLLDLQMILNGINNYKNPKLTRMLTAKFAMPKKATELKHLQCLEEELKPLEEVLNGAQSKNFHLRPRDLISNINVIVLELKGSETTFMCEYADETATIVEFLNRWITFSQSIISTLT

SEQ ID NO: 10 - protein, 133 aa, synthetic construct
  REGION 1..133: note = source = /note="Description of Artificial Sequence: Synthetic polypeptide"
APASSSTKKTQLQLEHLLLDLQMILNGINNYKNPKLTSMLTAKFAMPKKATELKHLQCLEEALKPLEEVLNLAQSKNFHLRPRDLISNINVIVLELKGSETTFMCEYADETATIVEFLNRWITFSQSIISTLT

SEQ ID NO: 11 - protein, 133 aa, synthetic construct
  REGION 1..133: note = source = /note="Description of Artificial Sequence: Synthetic polypeptide"
APTSSSTKKTQLQLEHLLLDLQMILNGINNYKNPKLTSMLTAKFAMPKKATELKHLQCLEEALKPLEEVLNLAQSKNFHLRPRDLISNINVIVLELKGSETTFMCEYADETATIVEFLNRWITFSQSIISTLT

SEQ ID NO: 12 - protein, 133 aa, synthetic construct
  REGION 1..133: note = source = /note="Description of Artificial Sequence: Synthetic polypeptide"
APASSSTKKTQLQLEHLLLDLQMILNGINNYKNPKLTSMLTAKFAMPKKATELKHLQCLEELLKPLEEVLNLAQSKNFHLRPRDLISNINVIVLELKGSETTFMCEYADETATIVEFLNRWITFSQSIISTLT

SEQ ID NO: 13 - protein, 133 aa, synthetic construct
  REGION 1..133: note = source = /note="Description of Artificial Sequence: Synthetic polypeptide"
APASSSTKKTQLQLEHLLLDLQMILNGINNYKNPKLTSMLTAKFAMPKKATELKHLQCLEELLKPLEVVLNLAQSKNFHLRPRDLISNINVIVLELKGSETTFMCEYADETATIVEFLNRWITFSQSIISTLT

SEQ ID NO: 14 - protein, 133 aa, synthetic construct
  REGION 1..133: note = source = /note="Description of Artificial Sequence: Synthetic polypeptide"
APASSSTKKTQLQLEHLLLDLQMILNGINNYKNPKLTSMLTIKFAMPKKATELKHLQCLEEALKPLEEVLNLAQSKNFHLRPRDLISNINVIVLELKGSETTFMCEYADETATIVEFLNRWITFSQSIISTLT

SEQ ID NO: 15 - protein, 133 aa, synthetic construct
  REGION 1..133: note = source = /note="Description of Artificial Sequence: Synthetic polypeptide"
APASSSTKKTQLQLEHLLLDLQMILNGINNYKNPKLTSMLTKKFAMPKKATELKHLQCLEEALKPLEEVLNLAQSKNFHLRPRDLISNINVIVLELKGSETTFMCEYADETATIVEFLNRWITFSQSIISTLT

SEQ ID NO: 16 - protein, 133 aa, synthetic construct
  REGION 1..133: note = source = /note="Description of Artificial Sequence: Synthetic polypeptide"
APASSSTKKTQLQLEHLLLDLQMILNGINNYKNPKLTSMLTAKFNMPKKATELKHLQCLEEALKPLEEVLNLAQSKNFHLRPRDLISNINVIVLELKGSETTFMCEYADETATIVEFLNRWITFSQSIISTLT

SEQ ID NO: 17 - protein, 133 aa, synthetic construct
  REGION 1..133: note = source = /note="Description of Artificial Sequence: Synthetic polypeptide"
APASSSTKKTQLQLEHLLLDLQMILNGINNYKNPKLTSMLTAKFRMPKKATELKHLQCLEEALKPLEEVLNLAQSKNFHLRPRDLISNINVIVLELKGSETTFMCEYADETATIVEFLNRWITFSQSIISTLT

SEQ ID NO: 18 - protein, 28 aa, synthetic construct
  REGION 1..28: note = source = /note="Description of Artificial Sequence: Synthetic peptide"
GGGGSGGGGSGGGGSLSGRSDNHGGGGS

SEQ ID NO: 19 - protein, 133 aa, synthetic construct
  REGION 1..133: note = source = /note="Description of Artificial Sequence: Synthetic polypeptide"
APASSSTKKTQLQLEHLLLDLQMILNGINNYKNPKLTSMLTKKFAMPKKATELKHLQCLEEALKPLEVVLNLAQSKNFHLRPRDLISNINVIVLELKGSETTFMCEYADETATIVEFLNRWITFSQSIISTLT

SEQ ID NO: 20 - protein, 133 aa, synthetic construct
  REGION 1..133: note = source = /note="Description of Artificial Sequence: Synthetic polypeptide"
APASSSTKKTQLQLEHLLLDLQMILNGINNYKNPKLTSMLTAKFNMPKKATELKHLQCLEEALKPLEVVLNLAQSKNFHLRPRDLISNINVIVLELKGSETTFMCEYADETATIVEFLNRWITFSQSIISTLT

SEQ ID NO: 21 - protein, 133 aa, synthetic construct
  REGION 1..133: note = source = /note="Description of Artificial Sequence: Synthetic polypeptide"
APASSSTKKTQLQLEHLLLDLQMILNGINNYKNPKLTSMLTAKFRMPKKATELKHLQCLEEALKPLEVVLNLAQSKNFHLRPRDLISNINVIVLELKGSETTFMCEYADETATIVEFLNRWITFSQSIISTLT

SEQ ID NO: 22 - protein, 133 aa, synthetic construct
  REGION 1..133: note = source = /note="Description of Artificial Sequence: Synthetic polypeptide"
APASSSTKKTQLQLEHLLLDLQMILNGINNYKNPKLTSMLTAKFAMPKKATELKHLQCLEEELKPLEEVLNLTQSKNFHLRPRDLISNINVIVLELKGSETTFMCEYADETATIVEFLNRWITFSQSIISTLT

SEQ ID NO: 23 - protein, 133 aa, synthetic construct
  REGION 1..133: note = source = /note="Description of Artificial Sequence: Synthetic polypeptide"
APASSSTKKTQLQLEHLLLDLQMILNGINNYKNPNLTSMLTAKFAMPKKATELKHLQCLEEELKPLEEVLNLTQSKNFHLRPRDLISNINVIVLELKGSETTFMCEYADETATIVEFLNRWITFSQSIISTLT

SEQ ID NO: 24 - protein, 133 aa, synthetic construct
  REGION 1..133: note = source = /note="Description of Artificial Sequence: Synthetic polypeptide"
APASSSTKKTQLQLEHLLLDLQMILNGINNYKNPKLTSMLTIKFAMPKKATELKHLQCLEEELKPLEEVLNLTQSKNFHLRPRDLISNINVIVLELKGSETTFMCEYADETATIVEFLNRWITFSQSIISTLT

SEQ ID NO: 25 - protein, 133 aa, synthetic construct
  REGION 1..133: note = source = /note="Description of Artificial Sequence: Synthetic polypeptide"
APASSSTKKTQLQLEHLLLDLQMILNGINNYKNPNLTSMLTIKFAMPKKATELKHLQCLEEELKPLEEVLNLTQSKNFHLRPRDLISNINVIVLELKGSETTFMCEYADETATIVEFLNRWITFSQSIISTLT

SEQ ID NO: 26 - protein, 133 aa, synthetic construct
  REGION 1..133: note = source = /note="Description of Artificial Sequence: Synthetic polypeptide"
APASSSTKKTQLQLEHLLLDLQMILNGINNYKNPKLTSMLTKKFAMPKKATELKHLQCLEEELKPLEEVLNLTQSKNFHLRPRDLISNINVIVLELKGSETTFMCEYADETATIVEFLNRWITFSQSIISTLT

SEQ ID NO: 27 - protein, 133 aa, synthetic construct
  REGION 1..133: note = source = /note="Description of Artificial Sequence: Synthetic polypeptide"
APASSSTKKTQLQLEHLLLDLQMILNGINNYKNPNLTSMLTKKFAMPKKATELKHLQCLEEELKPLEEVLNLTQSKNFHLRPRDLISNINVIVLELKGSETTFMCEYADETATIVEFLNRWITFSQSIISTLT

SEQ ID NO: 28 - protein, 133 aa, synthetic construct
  REGION 1..133: note = source = /note="Description of Artificial Sequence: Synthetic polypeptide"
APASSSTKKTQLQLEHLLLDLQMILNGINNYKNPKLTSMLTAKFNMPKKATELKHLQCLEEELKPLEEVLNLTQSKNFHLRPRDLISNINVIVLELKGSETTFMCEYADETATIVEFLNRWITFSQSIISTLT

SEQ ID NO: 29 - protein, 133 aa, synthetic construct
  REGION 1..133: note = source = /note="Description of Artificial Sequence: Synthetic polypeptide"
APASSSTKKTQLQLEHLLLDLQMILNGINNYKNPKLTSMLTAKFRMPKKATELKHLQCLEEELKPLEEVLNLTQSKNFHLRPRDLISNINVIVLELKGSETTFMCEYADETATIVEFLNRWITFSQSIISTLT

SEQ ID NO: 30 - protein, 133 aa, synthetic construct
  REGION 1..133: note = source = /note="Description of Artificial Sequence: Synthetic polypeptide"
APASSSTKKTQLQLEHLLLDLQMILNGINNYKNPKLTSMLTAKFAMPKKATELKHLQCLEEALKPLEEVLNLAQSKNFHLRPRDLISNINVIVLELKGSETTFMCEYADETATIVEFLNRWITFSWSIISTLT

SEQ ID NO: 31 - protein, 133 aa, synthetic construct
  REGION 1..133: note = source = /note="Description of Artificial Sequence: Synthetic polypeptide"
APASSSTKKTQLQLEHLLLDLQMILNGINNYKNPKLTSMLTKKFAMPKKATELKHLQCLEEALKPLEEVLNLTQSKNFHLRPRDLISNINVIVLELKGSETTFMCEYADETATIVEFLNRWITFSQSIISTLT

SEQ ID NO: 32 - protein, 133 aa, synthetic construct
  REGION 1..133: note = source = /note="Description of Artificial Sequence: Synthetic polypeptide"
APASSSTKKTQLQLEHLLLDLQMILNGINNYKNPKLTSMLTAKFNMPKKATELKHLQCLEEALKPLEEVLNLTQSKNFHLRPRDLISNINVIVLELKGSETTFMCEYADETATIVEFLNRWITFSQSIISTLT

SEQ ID NO: 33 - protein, 133 aa, synthetic construct
  REGION 1..133: note = source = /note="Description of Artificial Sequence: Synthetic polypeptide"
APASSSTKKTQLQLEHLLLDLQMILNGINNYKNPKLTSMLTAKFRMPKKATELKHLQCLEEALKPLEEVLNLTQSKNFHLRPRDLISNINVIVLELKGSETTFMCEYADETATIVEFLNRWITFSQSIISTLT

SEQ ID NO: 34 - protein, 16 aa, synthetic construct
  REGION 1..16: note = source = /note="Description of Artificial Sequence: Synthetic peptide"
GGSLSGRSDNHGGGGS

SEQ ID NO: 35 - protein, 13 aa, synthetic construct
  REGION 1..13: note = source = /note="Description of Artificial Sequence: Synthetic peptide"
GGSLSGRSDNHGS

SEQ ID NO: 36 - protein, 133 aa, synthetic construct
  REGION 1..133: note = source = /note="Description of Artificial Sequence: Synthetic polypeptide"
APASSSTKKTQLQLEHLLLDLQMILNGINNYKNPNLTSMLTAKFNMPKKATELKHLQCLEEELKPLEEVLNLTQSKNFHLRPRDLISNINVIVLELKGSETTFMCEYADETATIVEFLNRWITFSQSIISTLT

SEQ ID NO: 37 - protein, 133 aa, synthetic construct
  REGION 1..133: note = source = /note="Description of Artificial Sequence: Synthetic polypeptide"
APASSSTKKTQLQLEHLLLDLQMILNGINNYKNPNLTSMLTAKFRMPKKATELKHLQCLEEELKPLEEVLNLTQSKNFHLRPRDLISNINVIVLELKGSETTFMCEYADETATIVEFLNRWITFSQSIISTLT

SEQ ID NO: 38 - protein, 42 aa, synthetic construct
  REGION 1..42: note = source = /note="Description of Artificial Sequence: Synthetic polypeptide"
GGGGSGGGGSGGGGSISSGLLSSGGSGGSLSGRSDNHGGGGS

SEQ ID NO: 39 - protein, 133 aa, synthetic construct
  REGION 1..133: note = source = /note="Description of Artificial Sequence: Synthetic polypeptide"
APASSSTKKTQLQLEHLLLDLQMILNGINNYKNPNLTSMLTKKFAMPKKATELKHLQCLEEALKPLEEVLNLTQSKNFHLRPRDLISNINVIVLELKGSETTFMCEYADETATIVEFLNRWITFSQSIISTLT

SEQ ID NO: 40 - protein, 133 aa, synthetic construct
  REGION 1..133: note = source = /note="Description of Artificial Sequence: Synthetic polypeptide"
APASSSTKKTQLQLEHLLLDLQMILNGINNYKNPNLTSMLTAKFNMPKKATELKHLQCLEEALKPLEEVLNLTQSKNFHLRPRDLISNINVIVLELKGSETTFMCEYADETATIVEFLNRWITFSQSIISTLT

SEQ ID NO: 41 - protein, 133 aa, synthetic construct
  REGION 1..133: note = source = /note="Description of Artificial Sequence: Synthetic polypeptide"
APASSSTKKTQLQLEHLLLDLQMILNGINNYKNPNLTSMLTAKFRMPKKATELKHLQCLEEALKPLEEVLNLTQSKNFHLRPRDLISNINVIVLELKGSETTFMCEYADETATIVEFLNRWITFSQSIISTLT

SEQ ID NO: 42 - protein, 133 aa, synthetic construct
  REGION 1..133: note = source = /note="Description of Artificial Sequence: Synthetic polypeptide"
APASSSTKKTQLQLEHLLLDLQMILNGINNYKNPKLTAMLTAKFAMPKKATELKHLQCLEEALKPLEEVLNLAQSKNFHLRPRDLISNINVIVLELKGSETTFMCEYADETATIVEFLNRWITFSWSIISTLT

SEQ ID NO: 43 - protein, 133 aa, synthetic construct
  REGION 1..133: note = source = /note="Description of Artificial Sequence: Synthetic polypeptide"
APASSSTKKTQLQLEHLLLDLQMILNGINNYKNPKLTSMLTAKFAMPKKATELKHLQCLEEALKPLEEVLNLAQSKNFHLRPRDLISAINVIVLELKGSETTFMCEYADETATIVEFLNRWITFSHSIISTLT

SEQ ID NO: 44 - protein, 133 aa, synthetic construct
  REGION 1..133: note = source = /note="Description of Artificial Sequence: Synthetic polypeptide"
APASSSTKKTQLQLEHLLLDLQMILNGINNYKNPKLTSMLTAKFAMPKKATELKHLQCLEEALKPLEEVLNLAQSKNFHLRPRDLISNINVIVLELKGSETTFMCEYADETATIVEFLNRWITFSASIISTLT

SEQ ID NO: 45 - protein, 133 aa, synthetic construct
  REGION 1..133: note = source = /note="Description of Artificial Sequence: Synthetic polypeptide"
APASSSTKKTQLQLEHLLLDLQMILNGINNYKNPKLTSMLTKKFRMPKKATELKHLQCLEELLKPLEVVLNLAQSKNFHLRPRDLISNINVIVLELKGSETTFMCEYADETATIVEFLNRWITFSWSIISTLT

SEQ ID NO: 46 - protein, 133 aa, synthetic construct
  REGION 1..133: note = source = /note="Description of Artificial Sequence: Synthetic polypeptide"
APASSSTKKTQLQLEHLLLDLQMILNGINNYKNPKLTAMLTIKFNMPKKATELKHLQCLEELLKPLEVVLNLAQSKNFHLRPRDLISNINVIVLELKGSETTFMCEYADETATIVEFLNRWITFSWSIISTLT

SEQ ID NO: 47 - protein, 5 aa, synthetic construct
  REGION 1..5: note = source = /note="Description of Artificial Sequence: Synthetic peptide"
GGGGS

SEQ ID NO: 48 - protein, 10 aa, synthetic construct
  REGION 1..10: note = source = /note="Description of Artificial Sequence: Synthetic peptide"
GGGGSGGGGS

SEQ ID NO: 49 - protein, 15 aa, synthetic construct
  REGION 1..15: note = source = /note="Description of Artificial Sequence: Synthetic peptide"
GGGGSGGGGSGGGGS

SEQ ID NO: 50 - protein, 20 aa, synthetic construct
  VARIANT 10: note = /replace="Asn"
  SITE 1..20: note = /note="Variant residues given in the sequence have no preference with respect to those in the annotations for variant positions"
  REGION 1..20: note = source = /note="Description of Artificial Sequence: Synthetic peptide"
GGGGSGGGGAGGGGSGGGGS

SEQ ID NO: 51 - protein, 20 aa, synthetic construct
  VARIANT 10: note = /replace="Ala" or "Asn"
  VARIANT 15: note = /replace="Asn"
  SITE 1..20: note = /note="Variant residues given in the sequence have no preference with respect to those in the annotations for variant positions"
  REGION 1..20: note = source = /note="Description of Artificial Sequence: Synthetic peptide"
GGGGSGGGGSGGGGAGGGGS

SEQ ID NO: 52 - protein, 8 aa, synthetic construct
  REGION 1..8: note = source = /note="Description of Artificial Sequence: Synthetic peptide"
LSGRSDNH

SEQ ID NO: 53 - protein, 8 aa, synthetic construct
  REGION 1..8: note = source = /note="Description of Artificial Sequence: Synthetic peptide"
ISSGLLSS

SEQ ID NO: 54 - protein, 6 aa, synthetic construct
  REGION 1..6: note = source = /note="Description of Artificial Sequence: Synthetic peptide"
GPLGVR

SEQ ID NO: 55 - protein, 5 aa, synthetic construct
  REGION 1..5: note = source = /note="Description of Artificial Sequence: Synthetic peptide"
SGRSA

SEQ ID NO: 56 - protein, 42 aa, synthetic construct
  REGION 1..42: note = source = /note="Description of Artificial Sequence: Synthetic polypeptide"
GGGGSISSGLLSSGGSGGSLGGSGRSANAILEGGGGSGGGGS

SEQ ID NO: 57 - protein, 34 aa, synthetic construct
  REGION 1..34: note = source = /note="Description of Artificial Sequence: Synthetic polypeptide"
GGGGSISSGLLSSGGSLGGSGRSANAILEGGGGS

SEQ ID NO: 58 - protein, 37 aa, synthetic construct
  REGION 1..37: note = source = /note="Description of Artificial Sequence: Synthetic polypeptide"
GGGGSLGGSGRSANAILEGGSGGSISSGLLSSGGGGS

SEQ ID NO: 59 - protein, 34 aa, synthetic construct
  REGION 1..34: note = source = /note="Description of Artificial Sequence: Synthetic polypeptide"
GGGGSLGGSGRSANAILEGGSISSGLLSSGGGGS

SEQ ID NO: 60 - protein, 42 aa, synthetic construct
  REGION 1..42: note = source = /note="Description of Artificial Sequence: Synthetic polypeptide"
GGGGSLGGSGRSANAILEGGSGGSISSGLLSSGGGGSGGGGS

SEQ ID NO: 61 - protein, 33 aa, synthetic construct
  REGION 1..33: note = source = /note="Description of Artificial Sequence: Synthetic polypeptide"
GGGGSLGGSGRSANAILEGGGGSGGGGSGGGGS

SEQ ID NO: 62 - protein, 28 aa, synthetic construct
  REGION 1..28: note = source = /note="Description of Artificial Sequence: Synthetic peptide"
GGGGSGGGGSGGGGSISSGLLSSGGGGS

SEQ ID NO: 63 - protein, 18 aa, synthetic construct
  REGION 1..18: note = source = /note="Description of Artificial Sequence: Synthetic peptide"
GGGGSLSGRSDNHGGGGS

SEQ ID NO: 64 - protein, 48 aa, synthetic construct
  VARIANT 1..20: note = /note="This region may encompass 0, 1, 2, 3, or 4 'Gly Gly Gly Gly Ser' repeating units"
  VARIANT 29..48: note = /note="This region may encompass 0, 1, 2, 3, or 4 'Gly Gly Gly Gly Ser' repeating units"
  REGION 1..48: note = source = /note="Description of Artificial Sequence: Synthetic polypeptide"
GGGGSGGGGSGGGGSGGGGSGGWHTGRNGGGGSGGGGSGGGGSGGGGS

SEQ ID NO: 65 - protein, 48 aa, synthetic construct
  VARIANT 1..20: note = /note="This region may encompass 0, 1, 2, 3, or 4 'Gly Gly Gly Gly Ser' repeating units"
  VARIANT 29..48: note = /note="This region may encompass 0, 1, 2, 3, or 4 'Gly Gly Gly Gly Ser' repeating units"
  REGION 1..48: note = source = /note="Description of Artificial Sequence: Synthetic polypeptide"
GGGGSGGGGSGGGGSGGGGSTGRGPSWVGGGGSGGGGSGGGGSGGGGS

SEQ ID NO: 66 - protein, 48 aa, synthetic construct
  VARIANT 1..20: note = /note="This region may encompass 0, 1, 2, 3, or 4 'Gly Gly Gly Gly Ser' repeating units"
  VARIANT 29..48: note = /note="This region may encompass 0, 1, 2, 3, or 4 'Gly Gly Gly Gly Ser' repeating units"
  REGION 1..48: note = source = /note="Description of Artificial Sequence: Synthetic polypeptide"
GGGGSGGGGSGGGGSGGGGSSARGPSRWGGGGSGGGGSGGGGSGGGGS

SEQ ID NO: 67 - protein, 48 aa, synthetic construct
  VARIANT 1..20: note = /note="This region may encompass 0, 1, 2, 3, or 4 'Gly Gly Gly Gly Ser' repeating units"
  VARIANT 29..48: note = /note="This region may encompass 0, 1, 2, 3, or 4 'Gly Gly Gly Gly Ser' repeating units"
  REGION 1..48: note = source = /note="Description of Artificial Sequence: Synthetic polypeptide"
GGGGSGGGGSGGGGSGGGGSTARGPSFKGGGGSGGGGSGGGGSGGGGS

SEQ ID NO: 68 - protein, 47 aa, synthetic construct
  VARIANT 1..20: note = /note="This region may encompass 0, 1, 2, 3, or 4 'Gly Gly Gly Gly Ser' repeating units"
  VARIANT 28..47: note = /note="This region may encompass 0, 1, 2, 3, or 4 'Gly Gly Gly Gly Ser' repeating units"
  REGION 1..47: note = source = /note="Description of Artificial Sequence: Synthetic polypeptide"
GGGGSGGGGSGGGGSGGGGSTARGPSWGGGGSGGGGSGGGGSGGGGS

SEQ ID NO: 69 - protein, 48 aa, synthetic construct
  VARIANT 1..20: note = /note="This region may encompass 0, 1, 2, 3, or 4 'Gly Gly Gly Gly Ser' repeating units"
  VARIANT 29..48: note = /note="This region may encompass 0, 1, 2, 3, or 4 'Gly Gly Gly Gly Ser' repeating units"
  REGION 1..48: note = source = /note="Description of Artificial Sequence: Synthetic polypeptide"
GGGGSGGGGSGGGGSGGGGSLSGRSDNHGGGGSGGGGSGGGGSGGGGS

SEQ ID NO: 70 - protein, 59 aa, synthetic construct
  VARIANT 1..20: note = /note="This region may encompass 0, 1, 2, 3, or 4 'Gly Gly Gly Gly Ser' repeating units"
  VARIANT 40..59: note = /note="This region may encompass 0, 1, 2, 3, or 4 'Gly Gly Gly Gly Ser' repeating units"
  REGION 1..59: note = source = /note="Description of Artificial Sequence: Synthetic polypeptide"
GGGGSGGGGSGGGGSGGGGSLGGSGRSANAILEGPLGVRGGGGSGGGGSGGGGSGGGGS

SEQ ID NO: 71 - protein, 62 aa, synthetic construct
  VARIANT 1..20: note = /note="This region may encompass 0, 1, 2, 3, or 4 'Gly Gly Gly Gly Ser' repeating units"
  VARIANT 43..62: note = /note="This region may encompass 0, 1, 2, 3, or 4 'Gly Gly Gly Gly Ser' repeating units"
  REGION 1..62: note = source = /note="Description of Artificial Sequence: Synthetic polypeptide"
GGGGSGGGGSGGGGSGGGGSLGGSGRSANAILEGGSGPLGVRGGGGSGGGGSGGGGSGGGGS

SEQ ID NO: 72 - protein, 65 aa, synthetic construct
  VARIANT 1..20: note = /note="This region may encompass 0, 1, 2, 3, or 4 'Gly Gly Gly Gly Ser' repeating units"
  VARIANT 46..65: note = /note="This region may encompass 0, 1, 2, 3, or 4 'Gly Gly Gly Gly Ser' repeating units"
  REGION 1..65: note = source = /note="Description of Artificial Sequence: Synthetic polypeptide"
GGGGSGGGGSGGGGSGGGGSLGGSGRSANAILEGGSGGSGPLGVRGGGGSGGGGSGGGGSGGGGS

SEQ ID NO: 73 - protein, 56 aa, synthetic construct
  VARIANT 1..20: note = /note="This region may encompass 0, 1, 2, 3, or 4 'Gly Gly Gly Gly Ser' repeating units"
  VARIANT 37..56: note = /note="This region may encompass 0, 1, 2, 3, or 4 'Gly Gly Gly Gly Ser' repeating units"
  REGION 1..56: note = source = /note="Description of Artificial Sequence: Synthetic polypeptide"
GGGGSGGGGSGGGGSGGGGSISSGLLSSLSGRSDNHGGGGSGGGGSGGGGSGGGGS

SEQ ID NO: 74 - protein, 59 aa, synthetic construct
  VARIANT 1..20: note = /note="This region may encompass 0, 1, 2, 3, or 4 'Gly Gly Gly Gly Ser' repeating units"
  VARIANT 40..59: note = /note="This region may encompass 0, 1, 2, 3, or 4 'Gly Gly Gly Gly Ser' repeating units"
  REGION 1..59: note = source = /note="Description of Artificial Sequence: Synthetic polypeptide"
GGGGSGGGGSGGGGSGGGGSISSGLLSSGGSLSGRSDNHGGGGSGGGGSGGGGSGGGGS

SEQ ID NO: 75 - protein, 63 aa, synthetic construct
  VARIANT 1..20: note = /note="This region may encompass 0, 1, 2, 3, or 4 'Gly Gly Gly Gly Ser' repeating units"
  VARIANT 44..63: note = /note="This region may encompass 0, 1, 2, 3, or 4 'Gly Gly Gly Gly Ser' repeating units"
  REGION 1..63: note = source = /note="Description of Artificial Sequence: Synthetic polypeptide"
GGGGSGGGGSGGGGSGGGGSISSGLLSSGGSGGGSLSGRSDNHGGGGSGGGGSGGGGSGGGGS

SEQ ID NO: 76 - protein, 18 aa, synthetic construct
  REGION 1..18: note = source = /note="Description of Artificial Sequence: Synthetic peptide"
VNGGGGSGPLGVRAAQPA

SEQ ID NO: 77 - protein, 16 aa, synthetic construct
  REGION 1..16: note = source = /note="Description of Artificial Sequence: Synthetic peptide"
GGGGSGPLGVRGGGGS

SEQ ID NO: 78 - protein, 14 aa, synthetic construct
  REGION 1..14: note = source = /note="Description of Artificial Sequence: Synthetic peptide"
GGGGSGPLGVRGGS

SEQ ID NO: 79 - protein, 50 aa, synthetic construct
  VARIANT 1..20: note = /note="This region may encompass 0, 1, 2, 3, or 4 'Gly Gly Gly Gly Ser' repeating units"
  VARIANT 21..26: note = /note="This region may or may not be present in its entirety"
  VARIANT 31..50: note = /note="This region may encompass 0, 1, 2, 3, or 4 'Gly Gly Gly Gly Ser' repeating units"
  REGION 1..50: note = source = /note="Description of Artificial Sequence: Synthetic polypeptide"
GGGGSGGGGSGGGGSGGGGSQGQSGQPLGLGGGGSGGGGSGGGGSGGGGS

SEQ ID NO: 80 - protein, 54 aa, synthetic construct
  VARIANT 1..20: note = /note="This region may encompass 0, 1, 2, 3, or 4 'Gly Gly Gly Gly Ser' repeating units"
  VARIANT 21..26: note = /note="This region may or may not be present in its entirety"
  VARIANT 35..54: note = /note="This region may encompass 0, 1, 2, 3, or 4 'Gly Gly Gly Gly Ser' repeating units"
  REGION 1..54: note = source = /note="Description of Artificial Sequence: Synthetic polypeptide"
GGGGSGGGGSGGGGSGGGGSQGQSGQHTGRSGALGGGGSGGGGSGGGGSGGGGS

SEQ ID NO: 81 - protein, 54 aa, synthetic construct
  VARIANT 1..20: note = /note="This region may encompass 0, 1, 2, 3, or 4 'Gly Gly Gly Gly Ser' repeating units"
  VARIANT 21..26: note = /note="This region may or may not be present in its entirety"
  VARIANT 35..54: note = /note="This region may encompass 0, 1, 2, 3, or 4 'Gly Gly Gly Gly Ser' repeating units"
  REGION 1..54: note = source = /note="Description of Artificial Sequence: Synthetic polypeptide"
GGGGSGGGGSGGGGSGGGGSQGQSGQPLTGRSGGGGGGSGGGGSGGGGSGGGGS

SEQ ID NO: 82 - protein, 54 aa, synthetic construct
  VARIANT 1..20: note = /note="This region may encompass 0, 1, 2, 3, or 4 'Gly Gly Gly Gly Ser' repeating units"
  VARIANT 21..26: note = /note="This region may or may not be present in its entirety"
  VARIANT 35..54: note = /note="This region may encompass 0, 1, 2, 3, or 4 'Gly Gly Gly Gly Ser' repeating units"
  REGION 1..54: note = source = /note="Description of Artificial Sequence: Synthetic polypeptide"
GGGGSGGGGSGGGGSGGGGSQGQSGQAARGPAIHGGGGSGGGGSGGGGSGGGGS

SEQ ID NO: 83 - protein, 54 aa, synthetic construct
  VARIANT 1..20: note = /note="This region may encompass 0, 1, 2, 3, or 4 'Gly Gly Gly Gly Ser' repeating units"
  VARIANT 21..26: note = /note="This region may or may not be present in its entirety"
  VARIANT 35..54: note = /note="This region may encompass 0, 1, 2, 3, or 4 'Gly Gly Gly Gly Ser' repeating units"
  REGION 1..54: note = source = /note="Description of Artificial Sequence: Synthetic polypeptide"
GGGGSGGGGSGGGGSGGGGSQGQSGQRGPAFNPMGGGGSGGGGSGGGGSGGGGS

SEQ ID NO: 84 - protein, 54 aa, synthetic construct
  VARIANT 1..20: note = /note="This region may encompass 0, 1, 2, 3, or 4 'Gly Gly Gly Gly Ser' repeating units"
  VARIANT 21..26: note = /note="This region may or may not be present in its entirety"
  VARIANT 35..54: note = /note="This region may encompass 0, 1, 2, 3, or 4 'Gly Gly Gly Gly Ser' repeating units"
  REGION 1..54: note = source = /note="Description of Artificial Sequence: Synthetic polypeptide"
GGGGSGGGGSGGGGSGGGGSQGQSGQSSRGPAYLGGGGSGGGGSGGGGSGGGGS

SEQ ID NO: 85 - protein, 54 aa, synthetic construct
  VARIANT 1..20: note = /note="This region may encompass 0, 1, 2, 3, or 4 'Gly Gly Gly Gly Ser' repeating units"
  VARIANT 21..26: note = /note="This region may or may not be present in its entirety"
  VARIANT 35..54: note = /note="This region may encompass 0, 1, 2, 3, or 4 'Gly Gly Gly Gly Ser' repeating units"
  REGION 1..54: note = source = /note="Description of Artificial Sequence: Synthetic polypeptide"
GGGGSGGGGSGGGGSGGGGSQGQSGQRGPATPIMGGGGSGGGGSGGGGSGGGGS

SEQ ID NO: 86 - protein, 50 aa, synthetic construct
  VARIANT 1..20: note = /note="This region may encompass 0, 1, 2, 3, or 4 'Gly Gly Gly Gly Ser' repeating units"
  VARIANT 21..26: note = /note="This region may or may not be present in its entirety"
  VARIANT 31..50: note = /note="This region may encompass 0, 1, 2, 3, or 4 'Gly Gly Gly Gly Ser' repeating units"
  REGION 1..50: note = source = /note="Description of Artificial Sequence: Synthetic polypeptide"
GGGGSGGGGSGGGGSGGGGSQGQSGQRGPAGGGGSGGGGSGGGGSGGGGS

SEQ ID NO: 87 - protein, 56 aa, synthetic construct
  VARIANT 1..20: note = /note="This region may encompass 0, 1, 2, 3, or 4 'Gly Gly Gly Gly Ser' repeating units"
  VARIANT 21..26: note = /note="This region may or may not be present in its entirety"
  VARIANT 37..56: note = /note="This region may encompass 0, 1, 2, 3, or 4 'Gly Gly Gly Gly Ser' repeating units"
  REGION 1..56: note = source = /note="Description of Artificial Sequence: Synthetic polypeptide"
GGGGSGGGGSGGGGSGGGGSQGQSGQGGQPSGMWGWGGGGSGGGGSGGGGSGGGGS

SEQ ID NO: 88 - protein, 56 aa, synthetic construct
  VARIANT 1..20: note = /note="This region may encompass 0, 1, 2, 3, or 4 'Gly Gly Gly Gly Ser' repeating units"
  VARIANT 21..26: note = /note="This region may or may not be present in its entirety"
  VARIANT 37..56: note = /note="This region may encompass 0, 1, 2, 3, or 4 'Gly Gly Gly Gly Ser' repeating units"
  REGION 1..56: note = source = /note="Description of Artificial Sequence: Synthetic polypeptide"
GGGGSGGGGSGGGGSGGGGSQGQSGQFPRPLGITGLGGGGSGGGGSGGGGSGGGGS

SEQ ID NO: 89 - protein, 57 aa, synthetic construct
  VARIANT 1..20: note = /note="This region may encompass 0, 1, 2, 3, or 4 'Gly Gly Gly Gly Ser' repeating units"
  VARIANT 21..26: note = /note="This region may or may not be present in its entirety"
  VARIANT 38..57: note = /note="This region may encompass 0, 1, 2, 3, or 4 'Gly Gly Gly Gly Ser' repeating units"
  REGION 1..57: note = source = /note="Description of Artificial Sequence: Synthetic polypeptide"
GGGGSGGGGSGGGGSGGGGSQGQSGQVILMPLGFLGPGGGGSGGGGSGGGGSGGGGS

SEQ ID NO: 90 - protein, 54 aa, synthetic construct
  VARIANT 1..20: note = /note="This region may encompass 0, 1, 2, 3, or 4 'Gly Gly Gly Gly Ser' repeating units"
  VARIANT 21..26: note = /note="This region may or may not be present in its entirety"
  VARIANT 35..54: note = /note="This region may encompass 0, 1, 2, 3, or 4 'Gly Gly Gly Gly Ser' repeating units"
  REGION 1..54: note = source = /note="Description of Artificial Sequence: Synthetic polypeptide"
GGGGSGGGGSGGGGSGGGGSQGQSGQSPLTGRSGGGGGSGGGGSGGGGSGGGGS

SEQ ID NO: 91 - protein, 54 aa, synthetic construct
  VARIANT 1..20: note = /note="This region may encompass 0, 1, 2, 3, or 4 'Gly Gly Gly Gly Ser' repeating units"
  VARIANT 21..26: note = /note="This region may or may not be present in its entirety"
  VARIANT 35..54: note = /note="This region may encompass 0, 1, 2, 3, or 4 'Gly Gly Gly Gly Ser' repeating units"
  REGION 1..54: note = source = /note="Description of Artificial Sequence: Synthetic polypeptide"
GGGGSGGGGSGGGGSGGGGSQGQSGQSAGFSLPAGGGGSGGGGSGGGGSGGGGS

SEQ ID NO: 92 - protein, 55 aa, synthetic construct
  VARIANT 1..20: note = /note="This region may encompass 0, 1, 2, 3, or 4 'Gly Gly Gly Gly Ser' repeating units"
  VARIANT 21..26: note = /note="This region may or may not be present in its entirety"
  VARIANT 36..55: note = /note="This region may encompass 0, 1, 2, 3, or 4 'Gly Gly Gly Gly Ser' repeating units"
  REGION 1..55: note = source = /note="Description of Artificial Sequence: Synthetic polypeptide"
GGGGSGGGGSGGGGSGGGGSQGQSGQLAPLGLQRRGGGGSGGGGSGGGGSGGGGS

SEQ ID NO: 93 - protein, 54 aa, synthetic construct
  VARIANT 1..20: note = /note="This region may encompass 0, 1, 2, 3, or 4 'Gly Gly Gly Gly Ser' repeating units"
  VARIANT 21..26: note = /note="This region may or may not be present in its entirety"
  VARIANT 35..54: note = /note="This region may encompass 0, 1, 2, 3, or 4 'Gly Gly Gly Gly Ser' repeating units"
  REGION 1..54: note = source = /note="Description of Artificial Sequence: Synthetic polypeptide"
GGGGSGGGGSGGGGSGGGGSQGQSGQSGGPLGVRGGGGSGGGGSGGGGSGGGGS

SEQ ID NO: 94 - protein, 52 aa, synthetic construct
  VARIANT 1..20: note = /note="This region may encompass 0, 1, 2, 3, or 4 'Gly Gly Gly Gly Ser' repeating units"
  VARIANT 21..26: note = /note="This region may or may not be present in its entirety"
  VARIANT 33..52: note = /note="This region may encompass 0, 1, 2, 3, or 4 'Gly Gly Gly Gly Ser' repeating units"
  REGION 1..52: note = source = /note="Description of Artificial Sequence: Synthetic polypeptide"
GGGGSGGGGSGGGGSGGGGSQGQSGQGPLGVRGGGGSGGGGSGGGGSGGGGS

SEQ ID NO: 95 - protein, 54 aa, synthetic construct
  VARIANT 1..20: note = /note="This region may encompass 0, 1, 2, 3, or 4 'Gly Gly Gly Gly Ser' repeating units"
  VARIANT 21..26: note = /note="This region may or may not be present in its entirety"
  VARIANT 35..54: note = /note="This region may encompass 0, 1, 2, 3, or 4 'Gly Gly Gly Gly Ser' repeating units"
  REGION 1..54: note = source = /note="Description of Artificial Sequence: Synthetic polypeptide"
GGGGSGGGGSGGGGSGGGGSQGQSGQISSGLLSSGGGGSGGGGSGGGGSGGGGS

SEQ ID NO: 96 - protein, 54 aa, synthetic construct
  VARIANT 1..20: note = /note="This region may encompass 0, 1, 2, 3, or 4 'Gly Gly Gly Gly Ser' repeating units"
  VARIANT 21..26: note = /note="This region may or may not be present in its entirety"
  VARIANT 35..54: note = /note="This region may encompass 0, 1, 2, 3, or 4 'Gly Gly Gly Gly Ser' repeating units"
  REGION 1..54: note = source = /note="Description of Artificial Sequence: Synthetic polypeptide"
GGGGSGGGGSGGGGSGGGGSQGQSGQQNQALRMAGGGGSGGGGSGGGGSGGGGS

SEQ ID NO: 97 - protein, 54 aa, synthetic construct
  VARIANT 1..20: note = /note="This region may encompass 0, 1, 2, 3, or 4 'Gly Gly Gly Gly Ser' repeating units"
  VARIANT 21..26: note = /note="This region may or may not be present in its entirety"
  VARIANT 35..54: note = /note="This region may encompass 0, 1, 2, 3, or 4 'Gly Gly Gly Gly Ser' repeating units"
  REGION 1..54: note = source = /note="Description of Artificial Sequence: Synthetic polypeptide"
GGGGSGGGGSGGGGSGGGGSQGQSGQAQNLLGMVGGGGSGGGGSGGGGSGGGGS

SEQ ID NO: 98 - protein, 54 aa, synthetic construct
  VARIANT 1..20: note = /note="This region may encompass 0, 1, 2, 3, or 4 'Gly Gly Gly Gly Ser' repeating units"
  VARIANT 21..26: note = /note="This region may or may not be present in its entirety"
  VARIANT 35..54: note = /note="This region may encompass 0, 1, 2, 3, or 4 'Gly Gly Gly Gly Ser' repeating units"
  REGION 1..54: note = source = /note="Description of Artificial Sequence: Synthetic polypeptide"
GGGGSGGGGSGGGGSGGGGSQGQSGQSTFPFGMFGGGGSGGGGSGGGGSGGGGS

SEQ ID NO: 99 - protein, 54 aa, synthetic construct
  VARIANT 1..20: note = /note="This region may encompass 0, 1, 2, 3, or 4 'Gly Gly Gly Gly Ser' repeating units"
  VARIANT 21..26: note = /note="This region may or may not be present in its entirety"
  VARIANT 35..54: note = /note="This region may encompass 0, 1, 2, 3, or 4 'Gly Gly Gly Gly Ser' repeating units"
  REGION 1..54: note = source = /note="Description of Artificial Sequence: Synthetic polypeptide"
GGGGSGGGGSGGGGSGGGGSQGQSGQPVGYTSSLGGGGSGGGGSGGGGSGGGGS

SEQ ID NO: 100 - protein, 54 aa, synthetic construct
  VARIANT 1..20: note = /note="This region may encompass 0, 1, 2, 3, or 4 'Gly Gly Gly Gly Ser' repeating units"
  VARIANT 21..26: note = /note="This region may or may not be present in its entirety"
  VARIANT 35..54: note = /note="This region may encompass 0, 1, 2, 3, or 4 'Gly Gly Gly Gly Ser' repeating units"
  REGION 1..54: note = source = /note="Description of Artificial Sequence: Synthetic polypeptide"
GGGGSGGGGSGGGGSGGGGSQGQSGQDWLYWPGIGGGGSGGGGSGGGGSGGGGS

SEQ ID NO: 101 - protein, 54 aa, synthetic construct
  VARIANT 1..20: note = /note="This region may encompass 0, 1, 2, 3, or 4 'Gly Gly Gly Gly Ser' repeating units"
  VARIANT 21..26: note = /note="This region may or may not be present in its entirety"
  VARIANT 35..54: note = /note="This region may encompass 0, 1, 2, 3, or 4 'Gly Gly Gly Gly Ser' repeating units"
  REGION 1..54: note = source = /note="Description of Artificial Sequence: Synthetic polypeptide"
GGGGSGGGGSGGGGSGGGGSQGQSGQMIAPVAYRGGGGSGGGGSGGGGSGGGGS

SEQ ID NO: 102 - protein, 54 aa, synthetic construct
  VARIANT 1..20: note = /note="This region may encompass 0, 1, 2, 3, or 4 'Gly Gly Gly Gly Ser' repeating units"
  VARIANT 21..26: note = /note="This region may or may not be present in its entirety"
  VARIANT 35..54: note = /note="This region may encompass 0, 1, 2, 3, or 4 'Gly Gly Gly Gly Ser' repeating units"
  REGION 1..54: note = source = /note="Description of Artificial Sequence: Synthetic polypeptide"
GGGGSGGGGSGGGGSGGGGSQGQSGQRPSPMWAYGGGGSGGGGSGGGGSGGGGS

SEQ ID NO: 103 - protein, 54 aa, synthetic construct
  VARIANT 1..20: note = /note="This region may encompass 0, 1, 2, 3, or 4 'Gly Gly Gly Gly Ser' repeating units"
  VARIANT 21..26: note = /note="This region may or may not be present in its entirety"
  VARIANT 35..54: note = /note="This region may encompass 0, 1, 2, 3, or 4 'Gly Gly Gly Gly Ser' repeating units"
  REGION 1..54: note = source = /note="Description of Artificial Sequence: Synthetic polypeptide"
GGGGSGGGGSGGGGSGGGGSQGQSGQWATPRPMRGGGGSGGGGSGGGGSGGGGS

SEQ ID NO: 104 - protein, 54 aa, synthetic construct
  VARIANT 1..20: note = /note="This region may encompass 0, 1, 2, 3, or 4 'Gly Gly Gly Gly Ser' repeating units"
  VARIANT 21..26: note = /note="This region may or may not be present in its entirety"
  VARIANT 35..54: note = /note="This region may encompass 0, 1, 2, 3, or 4 'Gly Gly Gly Gly Ser' repeating units"
  REGION 1..54: note = source = /note="Description of Artificial Sequence: Synthetic polypeptide"
GGGGSGGGGSGGGGSGGGGSQGQSGQFRLLDWQWGGGGSGGGGSGGGGSGGGGS

SEQ ID NO: 105 - protein, 54 aa, synthetic construct
  VARIANT 1..20: note = /note="This region may encompass 0, 1, 2, 3, or 4 'Gly Gly Gly Gly Ser' repeating units"
  VARIANT 21..26: note = /note="This region may or may not be present in its entirety"
  VARIANT 35..54: note = /note="This region may encompass 0, 1, 2, 3, or 4 'Gly Gly Gly Gly Ser' repeating units"
  REGION 1..54: note = source = /note="Description of Artificial Sequence: Synthetic polypeptide"
GGGGSGGGGSGGGGSGGGGSQGQSGQLKAAPRWAGGGGSGGGGSGGGGSGGGGS

SEQ ID NO: 106 - protein, 54 aa, synthetic construct
  VARIANT 1..20: note = /note="This region may encompass 0, 1, 2, 3, or 4 'Gly Gly Gly Gly Ser' repeating units"
  VARIANT 21..26: note = /note="This region may or may not be present in its entirety"
  VARIANT 35..54: note = /note="This region may encompass 0, 1, 2, 3, or 4 'Gly Gly Gly Gly Ser' repeating units"
  REGION 1..54: note = source = /note="Description of Artificial Sequence: Synthetic polypeptide"
GGGGSGGGGSGGGGSGGGGSQGQSGQGPSHLVLTGGGGSGGGGSGGGGSGGGGS

SEQ ID NO: 107 - protein, 54 aa, synthetic construct
  VARIANT 1..20: note = /note="This region may encompass 0, 1, 2, 3, or 4 'Gly Gly Gly Gly Ser' repeating units"
  VARIANT 21..26: note = /note="This region may or may not be present in its entirety"
  VARIANT 35..54: note = /note="This region may encompass 0, 1, 2, 3, or 4 'Gly Gly Gly Gly Ser' repeating units"
  REGION 1..54: note = source = /note="Description of Artificial Sequence: Synthetic polypeptide"
GGGGSGGGGSGGGGSGGGGSQGQSGQLPGGLSPWGGGGSGGGGSGGGGSGGGGS

SEQ ID NO: 108 - protein, 54 aa, synthetic construct
  VARIANT 1..20: note = /note="This region may encompass 0, 1, 2, 3, or 4 'Gly Gly Gly Gly Ser' repeating units"
  VARIANT 21..26: note = /note="This region may or may not be present in its entirety"
  VARIANT 35..54: note = /note="This region may encompass 0, 1, 2, 3, or 4 'Gly Gly Gly Gly Ser' repeating units"
  REGION 1..54: note = source = /note="Description of Artificial Sequence: Synthetic polypeptide"
GGGGSGGGGSGGGGSGGGGSQGQSGQMGLFSEAGGGGGSGGGGSGGGGSGGGGS

SEQ ID NO: 109 - protein, 54 aa, synthetic construct
  VARIANT 1..20: note = /note="This region may encompass 0, 1, 2, 3, or 4 'Gly Gly Gly Gly Ser' repeating units"
  VARIANT 21..26: note = /note="This region may or may not be present in its entirety"
  VARIANT 35..54: note = /note="This region may encompass 0, 1, 2, 3, or 4 'Gly Gly Gly Gly Ser' repeating units"
  REGION 1..54: note = source = /note="Description of Artificial Sequence: Synthetic polypeptide"
GGGGSGGGGSGGGGSGGGGSQGQSGQSPLPLRVPGGGGSGGGGSGGGGSGGGGS

SEQ ID NO: 110 - protein, 54 aa, synthetic construct
  VARIANT 1..20: note = /note="This region may encompass 0, 1, 2, 3, or 4 'Gly Gly Gly Gly Ser' repeating units"
  VARIANT 21..26: note = /note="This region may or may not be present in its entirety"
  VARIANT 35..54: note = /note="This region may encompass 0, 1, 2, 3, or 4 'Gly Gly Gly Gly Ser' repeating units"
  REGION 1..54: note = source = /note="Description of Artificial Sequence: Synthetic polypeptide"
GGGGSGGGGSGGGGSGGGGSQGQSGQRMHLRSLGGGGGSGGGGSGGGGSGGGGS

SEQ ID NO: 111 - protein, 54 aa, synthetic construct
  VARIANT 1..20: note = /note="This region may encompass 0, 1, 2, 3, or 4 'Gly Gly Gly Gly Ser' repeating units"
  VARIANT 21..26: note = /note="This region may or may not be present in its entirety"
  VARIANT 35..54: note = /note="This region may encompass 0, 1, 2, 3, or 4 'Gly Gly Gly Gly Ser' repeating units"
  REGION 1..54: note = source = /note="Description of Artificial Sequence: Synthetic polypeptide"
GGGGSGGGGSGGGGSGGGGSQGQSGQLAAPLGLLGGGGSGGGGSGGGGSGGGGS

SEQ ID NO: 112 - protein, 54 aa, synthetic construct
  VARIANT 1..20: note = /note="This region may encompass 0, 1, 2, 3, or 4 'Gly Gly Gly Gly Ser' repeating units"
  VARIANT 21..26: note = /note="This region may or may not be present in its entirety"
  VARIANT 35..54: note = /note="This region may encompass 0, 1, 2, 3, or 4 'Gly Gly Gly Gly Ser' repeating units"
  REGION 1..54: note = source = /note="Description of Artificial Sequence: Synthetic polypeptide"
GGGGSGGGGSGGGGSGGGGSQGQSGQAVGLLAPPGGGGSGGGGSGGGGSGGGGS

SEQ ID NO: 113 - protein, 54 aa, synthetic construct
  VARIANT 1..20: note = /note="This region may encompass 0, 1, 2, 3, or 4 'Gly Gly Gly Gly Ser' repeating units"
  VARIANT 21..26: note = /note="This region may or may not be present in its entirety"
  VARIANT 35..54: note = /note="This region may encompass 0, 1, 2, 3, or 4 'Gly Gly Gly Gly Ser' repeating units"
  REGION 1..54: note = source = /note="Description of Artificial Sequence: Synthetic polypeptide"
GGGGSGGGGSGGGGSGGGGSQGQSGQLLAPSHRAGGGGSGGGGSGGGGSGGGGS

SEQ ID NO: 114 - protein, 54 aa, synthetic construct
  VARIANT 1..20: note = /note="This region may encompass 0, 1, 2, 3, or 4 'Gly Gly Gly Gly Ser' repeating units"
  VARIANT 21..26: note = /note="This region may or may not be present in its entirety"
  VARIANT 35..54: note = /note="This region may encompass 0, 1, 2, 3, or 4 'Gly Gly Gly Gly Ser' repeating units"
  REGION 1..54: note = source = /note="Description of Artificial Sequence: Synthetic polypeptide"
GGGGSGGGGSGGGGSGGGGSQGQSGQPAGLWLDPGGGGSGGGGSGGGGSGGGGS

SEQ ID NO: 115 - protein, 53 aa, synthetic construct
  VARIANT 1..20: note = /note="This region may encompass 0, 1, 2, 3, or 4 'Gly Gly Gly Gly Ser' repeating units"
  VARIANT 21..26: note = /note="This region may or may not be present in its entirety"
  VARIANT 34..53: note = /note="This region may encompass 0, 1, 2, 3, or 4 'Gly Gly Gly Gly Ser' repeating units"
  REGION 1..53: note = source = /note="Description of Artificial Sequence: Synthetic polypeptide"
GGGGSGGGGSGGGGSGGGGSQGQSGQISSGLSSGGGGSGGGGSGGGGSGGGGS

SEQ ID NO: 116 - protein, 62 aa, synthetic construct
  VARIANT 1..20: note = /note="This region may encompass 0, 1, 2, 3, or 4 'Gly Gly Gly Gly Ser' repeating units"
  VARIANT 43..62: note = /note="This region may encompass 0, 1, 2, 3, or 4 'Gly Gly Gly Gly Ser' repeating units"
  REGION 1..62: note = source = /note="Description of Artificial Sequence: Synthetic polypeptide"
GGGGSGGGGSGGGGSGGGGSISSGLLSSGGSGGSLSGRSDNHGGGGSGGGGSGGGGSGGGGS

SEQ ID NO: 117 - protein, 62 aa, synthetic construct
  VARIANT 1..20: note = /note="This region may encompass 0, 1, 2, 3, or 4 'Gly Gly Gly Gly Ser' repeating units"
  VARIANT 43..62: note = /note="This region may encompass 0, 1, 2, 3, or 4 'Gly Gly Gly Gly Ser' repeating units"
  REGION 1..62: note = source = /note="Description of Artificial Sequence: Synthetic polypeptide"
GGGGSGGGGSGGGGSGGGGSLSGRSDNHGGSGGSISSGLLSSGGGGSGGGGSGGGGSGGGGS

SEQ ID NO: 118 - protein, 54 aa, synthetic construct
  VARIANT 1..20: note = /note="This region may encompass 0, 1, 2, 3, or 4 'Gly Gly Gly Gly Ser' repeating units"
  VARIANT 21..26: note = /note="This region may or may not be present in its entirety"
  VARIANT 35..54: note = /note="This region may encompass 0, 1, 2, 3, or 4 'Gly Gly Gly Gly Ser' repeating units"
  REGION 1..54: note = source = /note="Description of Artificial Sequence: Synthetic polypeptide"
GGGGSGGGGSGGGGSGGGGSQGQSGQLSGRSDNHGGGGSGGGGSGGGGSGGGGS

SEQ ID NO: 119 - protein, 54 aa, synthetic construct
  VARIANT 1..20: note = /note="This region may encompass 0, 1, 2, 3, or 4 'Gly Gly Gly Gly Ser' repeating units"
  VARIANT 21..26: note = /note="This region may or may not be present in its entirety"
  VARIANT 35..54: note = /note="This region may encompass 0, 1, 2, 3, or 4 'Gly Gly Gly Gly Ser' repeating units"
  REGION 1..54: note = source = /note="Description of Artificial Sequence: Synthetic polypeptide"
GGGGSGGGGSGGGGSGGGGSQGQSGQTARGPSFKGGGGSGGGGSGGGGSGGGGS

SEQ ID NO: 120 - protein, 53 aa, synthetic construct
  VARIANT 1..20: note = /note="This region may encompass 0, 1, 2, 3, or 4 'Gly Gly Gly Gly Ser' repeating units"
  VARIANT 21..26: note = /note="This region may or may not be present in its entirety"
  VARIANT 34..53: note = /note="This region may encompass 0, 1, 2, 3, or 4 'Gly Gly Gly Gly Ser' repeating units"
  REGION 1..53: note = source = /note="Description of Artificial Sequence: Synthetic polypeptide"
GGGGSGGGGSGGGGSGGGGSQGQSGQTARGPSWGGGGSGGGGSGGGGSGGGGS

SEQ ID NO: 121 - protein, 54 aa, synthetic construct
  VARIANT 1..20: note = /note="This region may encompass 0, 1, 2, 3, or 4 'Gly Gly Gly Gly Ser' repeating units"
  VARIANT 21..26: note = /note="This region may or may not be present in its entirety"
  VARIANT 35..54: note = /note="This region may encompass 0, 1, 2, 3, or 4 'Gly Gly Gly Gly Ser' repeating units"
  REGION 1..54: note = source = /note="Description of Artificial Sequence: Synthetic polypeptide"
GGGGSGGGGSGGGGSGGGGSQGQSGQGGWHTGRNGGGGSGGGGSGGGGSGGGGS

SEQ ID NO: 122 - protein, 152 aa, Homo sapiens
DCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 123 - protein, 76 aa, Homo sapiens
TNDAEDCCLSVTQKPIPGYIVRNFHYLLIKDGCRVPAVVFTTLRGRQLCAPPDQPWVERIIQRLQRTSAKMKRRSS

SEQ ID NO: 124 - protein, 585 aa, Homo sapiens
DAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMRADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 125 - protein, 585 aa, synthetic construct
  REGION 1..585: note = source = /note="Description of Artificial Sequence: Synthetic polypeptide"
DAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMRADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGPKLVAASQAALGL

SEQ ID NO: 126 - protein, 733 aa, synthetic construct
  REGION 1..733: note = source = /note="Description of Artificial Sequence: Synthetic polypeptide"
DAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLGGGGSGGGGSGGGGSAPASSSTKKTQLQLEHLLLDLQMILNGINNYKNPKLTRMLTAKFAMPKKATELKHLQCLEEELKPLEEVLNGAQSKNFHLRPRDLISNINVIVLELKGSETTFMCEYADETATIVEFLNRWITFSQSIISTLT

SEQ ID NO: 127 - protein, 733 aa, synthetic construct
  REGION 1..733: note = source = /note="Description of Artificial Sequence: Synthetic polypeptide"
DAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLGGGGSGGGGSGGGGSAPASSSTKKTQLQLEHLLLDLQMILNGINNYKNPKLTSMLTAKFAMPKKATELKHLQCLEEALKPLEEVLNLAQSKNFHLRPRDLISNINVIVLELKGSETTFMCEYADETATIVEFLNRWITFSQSIISTLT

SEQ ID NO: 128 - protein, 733 aa, synthetic construct
  REGION 1..733: note = source = /note="Description of Artificial Sequence: Synthetic polypeptide"
DAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLGGGGSGGGGSGGGGSAPASSSTKKTQLQLEHLLLDLQMILNGINNYKNPKLTSMLTAKFAMPKKATELKHLQCLEEALKPLEEVLNLAQSKNFHLRPRDLISAINVIVLELKGSETTFMCEYADETATIVEFLNRWITFSHSIISTLT

SEQ ID NO: 129 - protein, 718 aa, synthetic construct
  REGION 1..718: note = source = /note="Description of Artificial Sequence: Synthetic polypeptide"
DAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLAPASSSTKKTQLQLEHLLLDLQMILNGINNYKNPKLTSMLTAKFAMPKKATELKHLQCLEEALKPLEEVLNLAQSKNFHLRPRDLISNINVIVLELKGSETTFMCEYADETATIVEFLNRWITFSWSIISTLT

SEQ ID NO: 130 - protein, 733 aa, synthetic construct
  REGION 1..733: note = source = /note="Description of Artificial Sequence: Synthetic polypeptide"
DAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLGGGGSGGGGSGGGGSAPASSSTKKTQLQLEHLLLDLQMILNGINNYKNPKLTSMLTAKFAMPKKATELKHLQCLEEALKPLEEVLNLAQSKNFHLRPRDLISNINVIVLELKGSETTFMCEYADETATIVEFLNRWITFSWSIISTLT

SEQ ID NO: 131 - protein, 718 aa, synthetic construct
  REGION 1..718: note = source = /note="Description of Artificial Sequence: Synthetic polypeptide"
DAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLAPASSSTKKTQLQLEHLLLDLQMILNGINNYKNPKLTSMLTAKFAMPKKATELKHLQCLEEALKPLEEVLNLAQSKNFHLRPRDLISNINVIVLELKGSETTFMCEYADETATIVEFLNRWITFSASIISTLT

SEQ ID NO: 132 - protein, 375 aa, synthetic construct
  REGION 1..375: note = source = /note="Description of Artificial Sequence: Synthetic polypeptide"
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLYCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSAPASSSTKKTQLQLEHLLLDLQMILNGINNYKNPKLTSMLTAKFAMPKKATELKHLQCLEEALKPLEEVLNLAQSKNFHLRPRDLISNINVIVLELKGSETTFMCEYADETATIVEFLNRWITFSQSIISTLT

SEQ ID NO: 133 - protein, 375 aa, synthetic construct
  REGION 1..375: note = source = /note="Description of Artificial Sequence: Synthetic polypeptide"
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLYCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSAPASSSTKKTQLQLEHLLLDLQMILNGINNYKNPKLTAMLTRKFKMPKKATELKHLQCLEEALKPLEEVLNLAQSKNFHLRPRDLISNINVIVLELKGSETTFMCEYADETATIVEFLNRWITFSQSIISTLT

SEQ ID NO: 134 - protein, 375 aa, synthetic construct
  REGION 1..375: note = source = /note="Description of Artificial Sequence: Synthetic polypeptide"
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSAPASSSTKKTQLQLEHLLLDLQMILNGINNYKNPKLTSMLTAKFAMPKKATELKHLQCLEEALKPLEEVLNLAQSKNFHLRPRDLISNINVIVLELKGSETTFMCEYADETATIVEFLNRWITFSHSIISTLT

SEQ ID NO: 135 - protein, 375 aa, synthetic construct
  REGION 1..375: note = source = /note="Description of Artificial Sequence: Synthetic polypeptide"
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSAPASSSTKKTQLQLEHLLLDLQMILNGINNYKNPKLTSMLTAKFAMPKKATELKHLQCLEEALKPLEEVLNLTQSKNFHLRPRDLISNINVIVLELKGSETTFMCEYADETATIVEFLNRWITFSQSIISTLT

SEQ ID NO: 136 - protein, 375 aa, synthetic construct; identical to SEQ ID NO: 135
  REGION 1..375: note = source = /note="Description of Artificial Sequence: Synthetic polypeptide"

SEQ ID NO: 137 - protein, 360 aa, synthetic construct
  REGION 1..360: note = source = /note="Description of Artificial Sequence: Synthetic polypeptide"
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKAPASSSTKKTQLQLEHLLLDLQMILNGINNYKNPKLTSMLTRKFAMPKKATELKHLQCLEEALKPLEEVLNLAQSKNFHLRPRDLISNINVIVLELKGSETTFMCEYADETATIVEFLNRWITFSQSIISTLT

SEQ ID NO: 138 - protein, 984 aa, synthetic construct
  REGION 1..984: note = source = /note="Description of Artificial Sequence: Synthetic polypeptide"
DAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGPKLVAASQAALGLGGGGSGGGGSLSGRSDNHGGSGGSAPASSSTKKTQLQLEHLLLDLQMILNGINNYKNPKLTSMLTAKFAMPKKATELKHLQCLEEALKPLEEVLNLAQSKNFHLRPRDLISNINVIVLELKGSETTFMCEYADETATIVEFLNRWITFSWSIISTLTGGGGSGGGGSGGGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 139 - protein, 375 aa, synthetic construct
  REGION 1..375: note = source = /note="Description of Artificial Sequence: Synthetic polypeptide"
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSAPASSSTKKTQLQLEHLLLDLQMILNGINNYKNPKLTRMLTAKFAMPKKATELKHLQCLEEALKPLEEVLNLAQSKNFHLRPRDLISEINVIVLELKGSETTFMCEYADETATIVEFLNRWITFSQSIISTLT

SEQ ID NO: 140 - protein, 375 aa, synthetic construct
  REGION 1..375: note = source = /note="Description of Artificial Sequence: Synthetic polypeptide"
APASSSTKKTQLQLEHLLLDLQMILNGINNYKNPKLTSMLTAKFAMPKKATELKHLQCLEEALKPLEEVLNLAQSKNFHLRPRDLISNINVIVLELKGSETTFMCEYADETATIVEFLNRWITFSWSIISTLTGGGGSGGGGSGGGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLYCLVKGFYPSDIAVEWESNGQPENNYKTTPPVLDSDGSFFLYSKLTVDKSRWQQGNVFSCSVMHEALHNHYTQKSLSLSPGK

SEQ ID NO: 141 - protein, 375 aa, synthetic construct
  REGION 1..375: note = source = /note="Description of Artificial Sequence: Synthetic polypeptide"
APASSSTKKTQLQLEHLLLDLQMILNGINNYKNPKLTSMLTAKFAMPKKATELKHLQCLEEALKPLEEVLNLAQSKNFHLRPRDLISNINVIVLELKGSETTFMCEYADETATIVEFLNRWITFSWSIISTLTGGGGSGGGGSGGGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 142 - protein, 378 aa, synthetic construct
  REGION 1..378: note = source = /note="Description of Artificial Sequence: Synthetic polypeptide"
AESKYGPPCPPCPAPEAAGGPSVFLFPPKPKDQLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHNHYTQKSLSLSLGKGGGGSGGGGSGGGGSAPASSSTKKTQLQLEHLLLDLQMILNGINNYKNPKLTSMLTAKFAMPKKATELKHLQCLEEALKPLEEVLNLAQSKNFHLRPRDLISNINVIVLELKGSETTFMCEYADETATIVEFLNRWITFSASIISTLT

SEQ ID NO: 143 - protein, 876 aa, synthetic construct
  REGION 1..876: note = source = /note="Description of Artificial Sequence: Synthetic polypeptide"
DKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGGSAPASSSTKKTQLQLEHLLLDLQMILNGINNYKNPKLTSMLTAKFAMPKKATELKHLQCLEEALKPLEEVLNLAQSKNFHLRPRDLISAINVIVLELKGSETTFMCEYADETATIVEFLNRWITFSHSIISTLTGGGGSTARGPSFKGGGGSAVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQGEFTTWSPWSQPLAFRTKPAALGKDTGGGGSGGGGSLSGRSDNHGGGGSGGGGSLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLQNLVIPWAPENLTLHKLSESQLELNWNNRFLNHCLEHLVQYRTDWDHSWTEQSVDYRHKFSLPSVDGQKRYTFRVRSRFNPLCGSAQHWSEWSHPIHWGSNTSKENPFLFALEA

SEQ ID NO: 144 - protein, 375 aa, synthetic construct
  REGION 1..375: note = source = /note="Description of Artificial Sequence: Synthetic polypeptide"
DKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLTSKLTVDKSRWQQGNVFSCSVMHEALHNHYTQKSLSLSPGKGGGGSGGGGSGGGGSAPASSSTKKTQLQLEHLLLDLQMILNGINNYKNPKLTSMLTAKFAMPKKATELKHLQCLEEALKPLEEVLNLAQSKNFHLRPRDLISNINVIVLELKGSETTFMCEYADETATIVEFLNRWITFSQSIISTLT

SEQ ID NO: 145 - protein, 459 aa, synthetic construct
  REGION 1..459: note = source = /note="Description of Artificial Sequence: Synthetic polypeptide"
DKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLYCLVKGFYPSDIAVEWESNGQPENNYKTTPPVLDSDGSFFLYSKLTVDKSRWQQGNVFSCSVMHEALHNHYTQKSLSLSPGKGGGGSTARGPSFKGGGGSAVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQGEFTTWSPWSQPLAFRTKPAALGKDT

SEQ ID NO: 146 - protein, 375 aa, synthetic construct
  REGION 1..375: note = source = /note="Description of Artificial Sequence: Synthetic polypeptide"
DKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLTSKLTVDKSRWQQGNVFSCSVMHEALHNHYTQKSLSLSPGKGGGGSGGGGSGGGGSAPASSSTKKTQLQLEHLLLDLQMILNGINNYKNPKLTSMLTAKFAMPKKATELKHLQCLEEALKPLEEVLNLAQSKNFHLRPRDLISNINVIVLELKGSETTFMCEYADETATIVEFLNRWITFSWSIISTLT

SEQ ID NO: 147 - protein, 727 aa, synthetic construct
  REGION 1..727: note = source = /note="Description of Artificial Sequence: Synthetic polypeptide"
DKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLYCLVKGFYPSDIAVEWESNGQPENNYKTTPPVLDSDGSFFLYSKLTVDKSRWQQGNVFSCSVMHEALHNHYTQKSLSLSPGKGGGGSTARGPSFKGGGGSAVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQGEFTTWSPWSQPLAFRTKPAALGKDTGGGGSGGGGSLSGRSDNHGGGGSGGGGSLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLQNLVIPWAPENLTLHKLSESQLELNWNNRFLNHCLEHLVQYRTDWDHSWTEQSVDYRHKFSLPSVDGQKRYTFRVRSRFNPLCGSAQHWSEWSHPIHWGSNTSKENPFLFALEA

SEQ ID NO: 148 - protein, 214 aa, synthetic construct
  REGION 1..214: note = source = /note="Description of Artificial Sequence: Synthetic polypeptide"
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 149 - protein, 451 aa, synthetic construct
  REGION 1..451: note = source = /note="Description of Artificial Sequence: Synthetic polypeptide"
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 150 - protein, 213 aa, synthetic construct
  REGION 1..213: note = source = /note="Description of Artificial Sequence: Synthetic polypeptide"
QIVLSQSPAILSASPGEKVTMTCRASSSVSYIHWFQQKPGSSPKPWIYATSNLASGVPVRFSGSGSGTSYSLTISRVEAEDAATYYCQQWTSNPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 151 - protein, 451 aa, synthetic construct
  REGION 1..451: note = source = /note="Description of Artificial Sequence: Synthetic polypeptide"
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKAEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 152 - protein, 218 aa, synthetic construct
  REGION 1..218: note = source = /note="Description of Artificial Sequence: Synthetic polypeptide"
DIVLTQSPASLAVSLGQRATISCKASQSVDFDGDSYMNWYQQKPGQPPKVLIYAASNLESGIPARFSGSGSGTDFTLNIHPVEEEDAATYYCQQSNEDPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 153 - protein, 446 aa, synthetic construct
  REGION 1..446: note = source = /note="Description of Artificial Sequence: Synthetic polypeptide"
QIQLQQSGPEVVKPGASVKISCKASGYTFTDYYITWVKQKPGQGLEWIGWIYPGSGNTKYNEKFKGKATLTVDTSSSTAFMQLSSLTSEDTAVYFCANYGNYWFAYWGQGTQVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 154 - protein, 214 aa, synthetic construct
  REGION 1..214: note = source = /note="Description of Artificial Sequence: Synthetic polypeptide"
DILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 155 - protein, 449 aa, synthetic construct
  REGION 1..449: note = source = /note="Description of Artificial Sequence: Synthetic polypeptide"
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 156 - protein, 214 aa, synthetic construct
  REGION 1..214: note = source = /note="Description of Artificial Sequence: Synthetic polypeptide"
DIQMTQSPSSLSASVGDRVTITCQASQDISNYLNWYQQKPGKAPKLLIYDASNLETGVPSRFSGSGSGTDFTFTISSLQPEDIATYFCQHFDHLPLAFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 157 - protein, 385 aa, synthetic construct
  REGION 1..385: note = source = /note="Description of Artificial Sequence: Synthetic polypeptide"
GHIYYSGNTNYNPSLKSRLTISIDTSKTQFSLKLSSVTAADTAIYYCVRDRVTGAFDIWGQGTMVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDERKCCVECPAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 158 - protein, 219 aa, synthetic construct
  REGION 1..219: note = source = /note="Description of Artificial Sequence: Synthetic polypeptide"
DIVMTQAAPSVPVTPGESVSISCRSSKSLLHSNGNTYLYWFLQRPGQSPQVLIYRMSNLASGVPDRFSGSGSGTAFTLRIRRVEAEDVGVYYCMQNLEYPFTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 159 - protein, 444 aa, synthetic construct
  REGION 1..444: note = source = /note="Description of Artificial Sequence: Synthetic polypeptide"
QVQLQQSGPELVKSGASVKMSCKASGNTLKDDHVHWVKQRPGQGLEWIGWIYPGGGRTRYNEKFKGKTTLTADKPSSTVNMLLSSLTSEDSAIYFCTNLVFDVWGAGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 160 - protein, 219 aa, synthetic construct
  REGION 1..219: note = source = /note="Description of Artificial Sequence: Synthetic polypeptide"
DVVMTQSPLSLPVTPGEPASISCRSSQSLVHSNANTYLHWYLQKPGQSPQLLIYKVSNRFSGVPDRFSGSGSGTDFTLKISRVEAEDVGVYYCSQNTHVPPTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 161 - protein, 445 aa, synthetic construct
  REGION 1..445: note = source = /note="Description of Artificial Sequence: Synthetic polypeptide"
QVQLVQSGAEVKKPGASVKVSCKASGYTFTDYEMHWVRQAPGQGLEWMGALDPKTGDTAYSQKFKGRVTLTADKSTSTAYMELSSLTSEDTAVYYCTRFYSYTYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 162 - protein, 220 aa, synthetic construct
  REGION 1..220: note = source = /note="Description of Artificial Sequence: Synthetic polypeptide"
DIVMTQSPSSLTVTAGEKVTMSCKSSQSLLNSGNQKNYLTWYQQKPGQPPKLLIYWASTRESGVPDRFTGSGSGTDFTLTISSVQAEDLAVYYCQNDYSYPFTFGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 163 - protein, 448 aa, synthetic construct
  REGION 1..448: note = source = /note="Description of Artificial Sequence: Synthetic polypeptide"
QVQLQQPGAELVRPGASVKLSCKASGYTFTSYWINWVKQRPGQGLEWIGNIYPSDSYTNYNQKFKDKATLTVDKSSSTAYMQLSSPTSEDSAVYYCTRSWRGNSFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 164 - protein, 11 aa, synthetic construct
  REGION 1..11: note = source = /note="Description of Artificial Sequence: Synthetic peptide"
KASQDVSIAVA

SEQ ID NO: 165 - protein, 7 aa, synthetic construct
  REGION 1..7: note = source = /note="Description of Artificial Sequence: Synthetic peptide"
SASYRYT

SEQ ID NO: 166 - protein, 9 aa, synthetic construct
  REGION 1..9: note = source = /note="Description of Artificial Sequence: Synthetic peptide"
QQHYITPLT

SEQ ID NO: 167 - protein, 5 aa, synthetic construct
  REGION 1..5: note = source = /note="Description of Artificial Sequence: Synthetic peptide"
NYGMN

SEQ ID NO: 168 - protein, 17 aa, synthetic construct
  REGION 1..17: note = source = /note="Description of Artificial Sequence: Synthetic peptide"
WINTYTGEPTYTDDFKG

SEQ ID NO: 169 - protein, 12 aa, synthetic construct
  REGION 1..12: note = source = /note="Description of Artificial Sequence: Synthetic peptide"
GGFGSSYWYFDV

SEQ ID NO: 170 - protein, 10 aa, synthetic construct
  REGION 1..10: note = source = /note="Description of Artificial Sequence: Synthetic peptide"
SASSSVSYMH

SEQ ID NO: 171 - protein, 7 aa, synthetic construct
  REGION 1..7: note = source = /note="Description of Artificial Sequence: Synthetic peptide"
DTSKLAS

SEQ ID NO: 172 - protein, 9 aa, synthetic construct
  REGION 1..9: note = source = /note="Description of Artificial Sequence: Synthetic peptide"
QQWSGYPLT

SEQ ID NO: 173 - protein, 5 aa, synthetic construct
  REGION 1..5: note = source = /note="Description of Artificial Sequence: Synthetic peptide"
GYTMN

SEQ ID NO: 174 - protein, 17 aa, synthetic construct
  REGION 1..17: note = source = /note="Description of Artificial Sequence: Synthetic peptide"
LITPYNGASSYNQKFRG

SEQ ID NO: 175 - protein, 10 aa, synthetic construct
  REGION 1..10: note = source = /note="Description of Artificial Sequence: Synthetic peptide"
GGYDGRGFDY

SEQ ID NO: 176 - protein, 110 aa, synthetic construct
  REGION 1..110: note = source = /note="Description of Artificial Sequence: Synthetic polypeptide"
GDIVMTQSQRFMSTSVGDRVSVTCKASQNVGTNVAWYQQKPGQSPKALIYSASYRYSGVPDRFTGSGSGTDFTLTISNVQSEDLAEYFCHQYYTYPLFTFGSGTKLEMKR

SEQ ID NO: 177 - protein, 121 aa, synthetic construct
  REGION 1..121: note = source = /note="Description of Artificial Sequence: Synthetic polypeptide"
QVKLQQSGPELKKPGETVKISCKASGYTFTVFGMNWVKQAPGKGLKWMGWINTKTGEATYVEEFKGRFAFSLETSATTAYLQINNLKNEDTAKYFCARWDFYDYVEAMDYWGQGTTVTVSS

SEQ ID NO: 178 - protein, 109 aa, synthetic construct
  REGION 1..109: note = source = /note="Description of Artificial Sequence: Synthetic polypeptide"
DIQMTQSPSSLSASVGDRVTITCKASQNVGTNVAWYQQKPGKAPKLLIYSASYRYSGVPSRFSGSGSGTDFTFTISSLQPEDIATYYCHQYYTYPLFTFGQGTKVEIKR

SEQ ID NO: 179 - protein, 121 aa, synthetic construct
  REGION 1..121: note = source = /note="Description of Artificial Sequence: Synthetic polypeptide"
QVQLVQSGSELKKPGASVKVSCKASGYTFTVFGMNWVRQAPGQGLEWMGWINTKTGEATYVEEFKGRFVFSLDTSVSTAYLQISSLKADDTAVYYCARWDFYDYVEAMDYWGQGTTVTVSS

SEQ ID NO: 180 - protein, 213 aa, synthetic construct
  REGION 1..213: note = source = /note="Description of Artificial Sequence: Synthetic polypeptide"
QIVLTQSPATLSLSPGERATLSCSASSGVNFMHWYQQKPGQAPRRLIFDTSKLASGIPARFSGSGSGTDYTLTISSLEPEDFAVYYCQQWSFNPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 181 - protein, 213 aa, synthetic construct
  REGION 1..213: note = source = /note="Description of Artificial Sequence: Synthetic polypeptide"
QIVLTQSPATLSLSPGERATLSCSASSGVNFMHWYQQKPGQAPKRLIFDTSKLASGVPARFSGSGSGTDYTLTISSLEPEDFAVYYCQQWSFNPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 182 - protein, 447 aa, synthetic construct
  REGION 1..447: note = source = /note="Description of Artificial Sequence: Synthetic polypeptide"
QVQLVQSGAEVKKPGASVKVSCKASGYTFTNNGINWLRQAPGQGLEWMGEIYPRSTNTLYAQKFQGRVTITADRSSNTAYMELSSLRSEDTAVYFCARTLTAPFAFWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 183 - protein, 113 aa, synthetic construct
  REGION 1..113: note = source = /note="Description of Artificial Sequence: Synthetic polypeptide"
DIVMTQSPDSLAVSLGERATINCKSSQSLLYSRNQKNYLAWYQQKPGQPPKLLIFWASTRESGVPDRFSGSGFGTDFTLTISSLQAEDVAVYYCQQYFSYPLTFGQGTKVEIK

SEQ ID NO: 184 - protein, 124 aa, synthetic construct
  REGION 1..124: note = source = /note="Description of Artificial Sequence: Synthetic polypeptide"
QVQLVQSGAEVKKPGASVKVSCKTSRYTFTEYTIHWVRQAPGQRLEWIGGINPNNGIPNYNQKFKGRVTITVDTSASTAYMELSSLRSEDTAVYYCARRRIAYGYDEGHAMDYWGQGTLVTVSS

SEQ ID NO: 185 - protein, 120 aa, synthetic construct
  REGION 1..120: note = source = /note="Description of Artificial Sequence: Synthetic polypeptide"
QVQLVQSGAEVKKPGASVKVSCKASGYSFTGYYMHWVKQSPGQGLEWIGRINPNNGVTLYNQKFKDRVTMTRDTSISTAYMELSRLRSDDTAVYYCARSTMITNYVMDYWGQGTLWTVSS

SEQ ID NO: 186 - protein, 120 aa, synthetic construct
  REGION 1..120: note = source = /note="Description of Artificial Sequence: Synthetic polypeptide"
QVQLVQSGAEVKKPGASVKVSCKASGYSFTGYYMHWVRQAPGQGLEWMGRINPNNGVTLYNQKFKDRVTMTRDTSISTAYMELSRLRSDDTAVYYCARSTMITNYVMDYWGQGTLVTVSS

SEQ ID NO: 187 - protein, 107 aa, synthetic construct
  REGION 1..107: note = source = /note="Description of Artificial Sequence: Synthetic polypeptide"
DIVMTQSPDSLAVSLGERATINCKASQSVSNDVAWYQQKPGQSPKLLISYTSSRYAGVPDRFSGSGSGTDFTLTISSLQAEDVAVYFCQQDYNSPPTFGGGTKLEIK

SEQ ID NO: 188 - protein, 107 aa, synthetic construct
  REGION 1..107: note = source = /note="Description of Artificial Sequence: Synthetic polypeptide"
DIVMTQSPDSLAVSLGERATINCKASQSVSNDVAWYQQKPGQPPKLLIYYTSSRYAGVPDRFSGSGSGTDFTLTISSLQAEDVAVYYCQQDYNSPPTFGGGTKLEIK

SEQ ID NO: 189 - protein, 214 aa, synthetic construct
  REGION 1..214: note = source = /note="Description of Artificial Sequence: Synthetic polypeptide"
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 190 - protein, 448 aa, synthetic construct
  REGION 1..448: note = source = /note="Description of Artificial Sequence: Synthetic polypeptide"
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 191 - protein, 593 aa, synthetic construct
  REGION 1..593: note = source = /note="Description of Artificial Sequence: Synthetic polypeptide"
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLYCLVKGFYPSDIAVEWESNGQPENNYKTTPPVLDSDGSFFLYSKLTVDKSRWQQGNVFSCSVMHEALHNHYTQKSLSLSPGKGGGSGGGSGGGSAPASSSTKKTQLQLEHLLLDLQMILNGINNYKNPKLTSMLTAKFAMPKKATELKHLQCLEEALKPLEEVLNLAQSKNFHLRPRDLISNINVIVLELKGSETTFMCEYADETATIVEFLNRWITFSQSIISTLT

SEQ ID NO: 192 - protein, 964 aa, synthetic construct
  REGION 1..964: note = source = /note="Description of Artificial Sequence: Synthetic polypeptide"
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLTSKLTVDKSRWQQGNVFSCSVMHEALHNHYTQKSLSLSPGKGGGGSGGGGSGGGGISSGLLSSGGSGGSLSGRSDNHGGGGSAVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQGEFTTWSPWSQPLAFRTKPAALGKDTGGGGSGGGGSGGGGSISSGLLSSGGSGGSLSGRSDNHGGGGSGGGGSAVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQGEFTTWSPWSQPLAFRTKPAALGKDT

SEQ ID NO: 193 - protein, 690 aa, synthetic construct
  REGION 1..690: note = source = /note="Description of Artificial Sequence: Synthetic polypeptide"
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLTSKLTVDKSRWQQGNVFSCSVMHEALHNHYTQKSLSLSPGKGGGGSGGGGSGGGGSLSGRSDNHGGGGSAVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQGEFTTWSPWSQPLAFRTKPAALGKDT

SEQ ID NO: 194 - protein, 227 aa, synthetic construct
  REGION 1..227: note = source = /note="Description of Artificial Sequence: Synthetic polypeptide"
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLTSKLTVDKSRWQQGNVFSCSVMHEALHNHYTQKSLSLSPGK

SEQ ID NO: 195 - protein, 469 aa, synthetic construct
  REGION 1..469: note = source = /note="Description of Artificial Sequence: Synthetic polypeptide"
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLTSKLTVDKSRWQQGNVFSCSVMHEALHNHYTQKSLSLSPGKGGGGSGGGGSGGGGSLSGRSDNHGGGGSAVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQGEFTTWSPWSQPLAFRTKPAALGKDT

SEQ ID NO: 196 - protein, 768 aa, synthetic construct
  REGION 1..768: note = source = /note="Description of Artificial Sequence: Synthetic polypeptide"
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLTSKLTVDKSRWQQGNVFSCSVMHEALHNHYTQKSLSLSPGKGGGGSGGGGSGGGGISSGLLSSGGSGGSLSGRSDNHGGGGSAVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQGEFTTWSPWSQPLAFRTKPAALGKDTGGGGSGGGGSGGGGSISSGLLSSGGSGGSLSGRSDNHGGGGSGGGGSLNTTILTPNGNEDTTADFFLTTMPTDSLSVSTLPLPEVQCFVFNVEYMNCTWNSSSEPQPTNLTLHYWYKNSDNDKVKCSHYLFSEEITSGCQLQKKEIHLYQTFVVQLQDPREPRRQATQMLKLQNLVIPWAPENLTLHKLSESQLELNWNNRFLNHCLEHLVQYRTDWDHSWTEQSVDYRHKFSLPSVDGQKRYTFRVRSRFNPLCGSAQHWSEWSHPIHWGSNTSKENPFLFALEA

SEQ ID NO: 197 - protein, 482 aa, synthetic construct
  REGION 1..482: note = source = /note="Description of Artificial Sequence: Synthetic polypeptide"
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLTSKLTVDKSRWQQGNVFSCSVMHEALHNHYTQKSLSLSPGKGGGGSGGGGSGGGGISSGLLSSGGSGGSLSGRSDNHGGGGSAVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQGEFTTWSPWSQPLAFRTKPAALGKDT

SEQ ID NO: 198 - protein, 743 aa, synthetic construct
  REGION 1..743: note = source = /note="Description of Artificial Sequence: Synthetic polypeptide"
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLTSKLTVDKSRWQQGNVFSCSVMHEALHNHYTQKSLSLSPGKGGGGSGGGGSGGGGISSGLLSSGGSGGSLSGRSDNHGGGGSAVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQGEFTTWSPWSQPLAFRTKPAALGKDTGGGGSGGGGSGGGGSISSGLLSSGGSGGSLSGRSDNHGGGGSGGGGSAVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQGEFTTWSPWSQPLAFRTKPAALGKDT

SEQ ID NO: 199 - protein, 482 aa, synthetic construct
  REGION 1..482: note = source = /note="Description of Artificial Sequence: Synthetic polypeptide"
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLTSKLTVDKSRWQQGNVFSCSVMHEALHNHYTQKSLSLSPGKGGGGSGGGGSGGGGISSGLLSSGGSGGSLSGRSDNHGGGGSAVNGTSQFTCFYNSYANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQGEFTTWSPWSQPLAFRTKPAALGKDT

SEQ ID NO: 200 - protein, 482 aa, synthetic construct
  REGION 1..482: note = source = /note="Description of Artificial Sequence: Synthetic polypeptide"
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLTSKLTVDKSRWQQGNVFSCSVMHEALHNHYTQKSLSLSPGKGGGGSGGGGSGGGGISSGLLSSGGSGGSLSGRSDNHGGGGSAVNGTSQFTCFYNSRANISCVWSQDGALQDTSCQVHAWPDRRRWNQTCELLPVSQASWACNLILGAPESQKLTTVDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQGEFTTWSPWSQPLAFRTKPAALGKDT

SEQ ID NO: 201 - protein, 482 aa, synthetic construct
  REGION 1..482: note = source = /note="Description of Artificial Sequence: Synthetic polypeptide"
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLTSKLTVDKSRWQQGNVFSCSVMHEALHNHYTQKSLSLSPGKGGGGSGGGGSGGGGISSGLLSSGGSGGSLSGRSDNHGGGGSAVNGTSQFTCFYNSRANISCVWSQDGALQDTSCQVHAWPDRRRWNQTCELLPVSQASWACNLILGAPDHQKLTTVDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQGEFTTWSPWSQPLAFRTKPAALGKDT

SEQ ID NO: 202 - protein, 482 aa, synthetic construct
  REGION 1..482: note = source = /note="Description of Artificial Sequence: Synthetic polypeptide"
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLTSKLTVDKSRWQQGNVFSCSVMHEALHNHYTQKSLSLSPGKGGGGSGGGGSGGGGISSGLLSSGGSGGSLSGRSDNHGGGGSAVNGTSQFTCFYNSRANISCVWSQDGALQDTSCQVHAWPDRRRWNQTCELLPVSQASWACNLILGAPDSQKLTTQDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQGEFTTWSPWSQPLAFRTKPAALGKDT

SEQ ID NO: 203 - protein, 482 aa, synthetic construct
  REGION 1..482: note = source = /note="Description of Artificial Sequence: Synthetic polypeptide"
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLTSKLTVDKSRWQQGNVFSCSVMHEALHNHYTQKSLSLSPGKGGGGSGGGGSGGGGISSGLLSSGGSGGSLSGRSDNHGGGGSAVNGTSQFTCFYNSRANISCVWSQDGALQDTSCQVHAWPDRRRWNQTCELLPVSQASWACNLILGAPDSQKLTTFDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQGEFTTWSPWSQPLAFRTKPAALGKDT

SEQ ID NO: 204 - protein, 482 aa, synthetic construct
  REGION 1..482: note = source = /note="Description of Artificial Sequence: Synthetic polypeptide"
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLTSKLTVDKSRWQQGNVFSCSVMHEALHNHYTQKSLSLSPGKGGGGSGGGGSGGGGISSGLLSSGGSGGSLSGRSDNHGGGGSAVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFQRHLEFEARTLSPGHTWEEAPLLTLKQKQEWICLETLTPDTQYEFQVRVKPLQGEFTTWSPWSQPLAFRTKPAALGKDT

SEQ ID NO: 205 - protein, 482 aa, synthetic construct
  REGION 1..482: note = source = /note="Description of Artificial Sequence: Synthetic polypeptide"
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLTSKLTVDKSRWQQGNVFSCSVMHEALHNHYTQKSLSLSPGKGGGGSGGGGSGGGGISSGLLSSGGSGGSLSGRSDNHGGGGSAVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFQRRLEFEARTLSPGHTWEEAPLLTLKQKQEWICLETLTPDTQYEFQVRVKPLQGEFTTWSPWSQPLAFRTKPAALGKDT

SEQ ID NO: 206 - protein, 697 aa, synthetic construct
  REGION 1..697: note = source = /note="Description of Artificial Sequence: Synthetic polypeptide"
MGWTLVFLFLLSVTAGVHSEVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLTSKLTVDKSRWQQGNVFSCSVMHEALHNHYTQKSLSLSPGKGGSLSGRSDNHGGGGSAVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQGEFTTWSPWSQPLAFRTKPAALGKDT

SEQ ID NO: 207 - protein, 694 aa, synthetic construct
  REGION 1..694: note = source = /note="Description of Artificial Sequence: Synthetic polypeptide"
MGWTLVFLFLLSVTAGVHSEVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLTSKLTVDKSRWQQGNVFSCSVMHEALHNHYTQKSLSLSPGKGGSLSGRSDNHGSAVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQGEFTTWSPWSQPLAFRTKPAALGKDT

SEQ ID NO: 208 - protein, 694 aa, synthetic construct
  REGION 1..694: note = source = /note="Description of Artificial Sequence: Synthetic polypeptide"
MGWTLVFLFLLSVTAGVHSEVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLTSKLTVDKSRWQQGNVFSCSVMHEALHNHYTQKSLSLSPGKGGSLSGRSDNHGSAVNGTSQFTCFYNSRANISCVWSQDGALQDTSCQVHAWPDRRRWNQTCELLPVSQASWACNLILGAPESQKLTTVDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQGEFTTWSPWSQPLAFRTKPAALGKDT

SEQ ID NO: 209 - protein, 597 aa, synthetic construct
  REGION 1..597: note = source = /note="Description of Artificial Sequence: Synthetic polypeptide"
EVQLVESGGGLVKPGGSLRLSCAVSGFYFNRGYWICWVRQAPGKGLEWIGCIDTGSGVPYYANWAKGRFTISRHTSKTTLTLQMNSLRAEDTASYFCARNSDSIYFNLWGPGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLYCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSAPASSSTKKTQLQLEHLLLDLQMILNGINNYKNPKLTSMLTAKFAMPKKATELKHLQCLEEALKPLEEVLNLAQSKNFHLRPRDLISNINVIVLELKGSETTFMCEYADETATIVEFLNRWITFSQSIISTLT

SEQ ID NO: 210 - protein, 710 aa, synthetic construct
  REGION 1..710: note = source = /note="Description of Artificial Sequence: Synthetic polypeptide"
EVQLVESGGGLVKPGGSLRLSCAVSGFYFNRGYWICWVRQAPGKGLEWIGCIDTGSGVPYYANWAKGRFTISRHTSKTTLTLQMNSLRAEDTASYFCARNSDSIYFNLWGPGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLTSKLTVDKSRWQQGNVFSCSVMHEALHNHYTQKSLSLSPGKGGGGSGGGGSGGGGSISSGLLSSGGSGGSLSGRSDNHGGGGSGGGGSAVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQGEFTTWSPWSQPLAFRTKPAALGKDT

SEQ ID NO: 211 - protein, 678 aa, synthetic construct
  REGION 1..678: note = source = /note="Description of Artificial Sequence: Synthetic polypeptide"
EVQLVESGGGLVKPGGSLRLSCAVSGFYFNRGYWICWVRQAPGKGLEWIGCIDTGSGVPYYANWAKGRFTISRHTSKTTLTLQMNSLRAEDTASYFCARNSDSIYFNLWGPGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLTSKLTVDKSRWQQGNVFSCSVMHEALHNHYTQKSLSLSPGKGGGGSGGGGSGGGGSAVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQGEFTTWSPWSQPLAFRTKPAALGKDT

SEQ ID NO: 212 - protein, 691 aa, synthetic construct
  REGION 1..691: note = source = /note="Description of Artificial Sequence: Synthetic polypeptide"
EVQLVESGGGLVKPGGSLRLSCAVSGFYFNRGYWICWVRQAPGKGLEWIGCIDTGSGVPYYANWAKGRFTISRHTSKTTLTLQMNSLRAEDTASYFCARNSDSIYFNLWGPGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLTSKLTVDKSRWQQGNVFSCSVMHEALHNHYTQKSLSLSPGKGGGGSLSGRSDNHGGGGSGGGGSGGGGSAVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQGEFTTWSPWSQPLAFRTKPAALGKDT

SEQ ID NO: 213 - protein, 691 aa, synthetic construct
  REGION 1..691: note = source = /note="Description of Artificial Sequence: Synthetic polypeptide"
EVQLVESGGGLVKPGGSLRLSCAVSGFYFNRGYWICWVRQAPGKGLEWIGCIDTGSGVPYYANWAKGRFTISRHTSKTTLTLQMNSLRAEDTASYFCARNSDSIYFNLWGPGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLTSKLTVDKSRWQQGNVFSCSVMHEALHNHYTQKSLSLSPGKGGGGSGGGGSGGGGSISSGLLSSGGGGSAVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQGEFTTWSPWSQPLAFRTKPAALGKDT

SEQ ID NO: 214 - protein, 898 aa, synthetic construct
  REGION 1..898: note = source = /note="Description of Artificial Sequence: Synthetic polypeptide"
DIQMTQSPSSVSASVGDRVTITCQASQSIGGYLSWYQQKPGQPPKLLIYKASTLASGVPSRFKGSGSGTDFTLTISSLDSEDAATYYCQNYAGVSIYGAVFGGGTKVVVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECEVQLVESGGGLVKPGGSLRLSCAVSGFYFNRGYWICWVRQAPGKGLEWIGCIDTGSGVPYYANWAKGRFTISRHTSKTTLTLQMNSLRAEDTASYFCARNSDSIYFNLWGPGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLTSKLTVDKSRWQQGNVFSCSVMHEALHNHYTQKSLSLSPGKGGGGSLSGRSDNHGGGGSAVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQGEFTTWSPWSQPLAFRTKPAALGKDT

SEQ ID NO: 215 - protein, 681 aa, synthetic construct
  REGION 1..681: note = source = /note="Description of Artificial Sequence: Synthetic polypeptide"
EVQLVESGGGLVKPGGSLRLSCAVSGFYFNRGYWICWVRQAPGKGLEWIGCIDTGSGVPYYANWAKGRFTISRHTSKTTLTLQMNSLRAEDTASYFCARNSDSIYFNLWGPGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLTSKLTVDKSRWQQGNVFSCSVMHEALHNHYTQKSLSLSPGKGGGGSLSGRSDNHGGGGSAVNGTSQFTCFYNSRANISCVWSQDGALQDTSCQVHAWPDRRRWNQTCELLPVSQASWACNLILGAPESQKLTTVDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQGEFTTWSPWSQPLAFRTKPAALGKDT

SEQ ID NO: 216 - protein, 217 aa, synthetic construct
  REGION 1..217: note = source = /note="Description of Artificial Sequence: Synthetic polypeptide"
DIQMTQSPSSVSASVGDRVTITCQASQSIGGYISWYQQKPGQPPKLLIYKASTLASGVPSRFKGSGSGTDFTLTISSLDSEDAATYYCQNYAGVSIYGAVFGGGTKVVVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 217 - protein, 47 aa, synthetic construct
  REGION 1..47: note = source = /note="Description of Artificial Sequence: Synthetic polypeptide"
GGGGSGGGGSGGGGSISSGLLSSGGSGGSLSGRSDNHGGGGSGGGGS